(19) United States
(12) Patent Application Publication
(10) Pub. No.: US 2025/0339096 A1
(43) Pub. Date: Nov. 6, 2025

(54) Hydration Assessment System

(71) Applicant: Medici Technologies LLC, Albuquerque, NM (US)

(72) Inventor: Mark Ries Robinson, Albuquerque, NM (US)

(21) Appl. No.: 19/264,274

(22) Filed: Jul. 9, 2025

Related U.S. Application Data
(63) Continuation-in-part of application No. 18/421,071, filed on Jan. 24, 2024, now Pat. No. 12,369,855.
Application No. 18/421,071 is a continuation of application No. 17/049,508, filed on Oct. 21, 2020, now Pat. No. 11,471,102.
(60) Provisional application No. 62/864,465, filed on Jun. 20, 2019.

(51) Int. Cl. A61B 5/00; A61B 5/02; A61B 5/0205; A61B 5/024; A61B 5/11; G16H 40/63

(52) U.S. Cl. CPC A61B 5/4875; A61B 5/02028; A61B 5/0205; A61B 5/486; A61B 5/6826; A61B 5/6843; A61B 5/7278; A61B 5/7282; G16H 40/63; A61B 5/02416; A61B 5/1116; A61B 5/7405; A61B 5/742; A61B 5/7455; A61B 2562/0219

(57) ABSTRACT

The present invention provides methods and systems that provide for reliable, convenient, and noninvasive assessment of hydration status. The methods and apparatuses can use the temporal sequence of the aortic value opening and closing along with the user's body position to derive parameters that determine the hydration status of the user. The user can use this information to make near-term lifestyle changes that can improve physical performance, health, and general wellbeing.

### TECHNICAL FIELD

The present invention relates to determination of an individual's hydration status, and in particular to wearable, noninvasive systems that can determine an individual's hydration status.

### BACKGROUND

The determination of an individual's hydration status in a convenient fashion is a desired objective for athletes, general consumers, and elderly individuals.

As reviewed by Jequier and Constant (2010), water is a vital constituent of the human body, and sufficient hydration is critical to overall physiological performance due to the impact of vascular volume on cognitive function, kidney function and cardiovascular function. Jequier, E., & Constant, F. (2010). Water as an essential nutrient: the physiological basis of hydration. European journal of clinical nutrition, 64(2), 115.

Dehydration (body water deficit) is a physiologic state that can have profound implications for human health and performance. FIG. 1 displays the mental and physical effects of dehydration as a function of the percent of weight lost in water. Early effects include irritability and decrease of peak physical performance, while severe effects include coma and ultimately death. Unfortunately, dehydration can be difficult to assess and there is no universal gold standard. As illustrated in FIG. 2, roughly 60% of the human body is composed of water, which is contained in different intracellular and extracellular compartments. The complexity of hydration measurement arises, in part, from the ubiquitous presence of water in multiple body compartments, and the continuous homeostatic flux between compartments. Armstrong (2007) speaks to the complexity of (1) accurately defining hydration due to the multiple water compartments and (2) the measurement difficulties of any individual compartment. Armstrong, L. E. (2007). Assessing hydration status: the elusive gold standard. Journal of the American College of Nutrition, 26, 575S-584S.

Through the activities of daily living, an individual creates a “daily water deficit”, i.e., the amount of water that a person needs to consume to compensate for loss due to sweat, urine, and respiration, etc. Depending on individual size and activity level, daily water deficit varies from about 1 L to 6 L. This deficit must be replenished, largely through oral intake. Armstrong, Lawrence E. “Assessing hydration status: the elusive gold standard.” Journal of the American College of Nutrition 26.sup5 (2007): 575S-584S.

The large degree of variance in daily water requirements limits the use of standard tables or “rules of thumb” to guide oral replenishment. Perspiration rate, in particular, varies significantly between individuals and within individuals, and depends on factors including base physiology, activity intensity, environmental temperature and humidity, and the amount and type of clothing or equipment worn. Thus, the same individual can have strikingly different sweat rates and overall water loss for the same activity on different days.

Our innate mechanism for guiding rehydration, the “drink to thirst” mechanism, is typically effective under conditions with limited physiological perturbations or challenges. However, under conditions of physiological stress, the “drink to thirst” response has been shown to ineffective. A recent paper in Medicine & Science in Sports & Exercise by Stavros Kavouras at the University of Arkansas showed that endurance cyclists experienced performance declines when hydrating by thirst instead of following a predetermined hydration schedule. Adams, J. D., et al. “Dehydration Impairs Cycling Performance, Independently of Thirst: A Blinded Study.” Medicine and science in sports and exercise 50.8 (2018): 1697-1703.

U.S. Army researcher Robert Kenefick, who wrote that: “planned drinking is optimal in longer duration activities of greater than 90 minutes, particularly in the heat; higher-intensity exercise with high sweat rates; exercise where performance is a concern; and when carbohydrate intake of 1 gram/minute is desired.” He also pointed to 60-90 minute timeframes as a subjective gray zone, with no clear evidence in favor of hydration by thirst, or by schedule, (Kenefick, Robert W. “Drinking strategies: planned drinking versus drinking to thirst.” Sports Medicine 48.1 (2018): 31-37.).

Sufficient hydration is critical for optimal physical performance because dehydration directly affects the volume of extracellular fluid within the vascular space, known as the plasma volume. Reductions in plasma volume decrease the amount of blood entering the heart during diastole, the phase in the cardiac cycle where the heart relaxes and fills with blood. Less blood entering the heart during diastole decreases end diastolic volume and thus the amount of blood leaving the heart during systole, the phase where the heart contracts. The result is a decreased stroke volume, cardiac output, and maximal aerobic power (VO2 max).

Current hydration assessment techniques include (1) total body water as measured by isotope dilution or estimated by bioelectrical impedance analysis, (2) plasma markers, such as osmolality, sodium, hematocrit and hemoglobin changes, or the concentrations of hormones that help regulate body fluids, (3) urine markers, such as osmolality, specific gravity, or color, and (4) observable physical signs, such as salivary flow or gross, physical signs and symptoms of clinical dehydration. The majority of these methods require clinical equipment and/or expertise and are far from convenient.

Thus, the ability to conveniently access overall hydration status at multiple points throughout the day has significant value, particularly for individuals undergoing physiological stress who cannot rely on their thirst mechanism to guide rehydration.

Sports-science consensus holds that endurance performance and thermoregulation begin to suffer once dehydration exceeds about 2% of whole-body mass (BML), so many practitioners treat a 1% BML drop as an “early-warning” target that allows athletes or patients to intervene before harm occurs. Because the water fraction of the human body typically falls anywhere between roughly 45% and 65% of total mass, with population averages clustering around the mid-50s, a 1% loss of body weight translates to an absolute water deficit of about 1.5-2.2% of total-body water (TBW), the more physiologically relevant metric. Designing a sensor that can reliably resolve changes on the order of about 2% TBW defines a practical accuracy threshold implied by the 1% BML early-warning criterion.

U.S. Pat. No. 5,964,701 to Asada et al., entitled “Patient Monitoring Finger Ring Sensor”, discloses a health status monitor incorporated into a finger ring, comprising sensors that may include a thermocouple for measuring skin temperature, an electrical impedance plethysmograph, and one or more optical sensors for pulse rate and measurements of blood constituent concentration and blood flow. U.S. Pat. No. 6,402,690 B1 to Rhee et al., entitled “Isolating Ring Sensor Design”, discloses a heath monitoring system for a patient by performing measurements such as skin temperature, blood flow, blood constituent concentration, and pulse rate at the finger of the patient. The monitoring system has an inner ring proximate to the finger as well as an outer ring, mechanically decoupled from the inner ring, that shields the inner ring from external loads. US Patent application publication 2016/0166161 A1 by Yang et al., entitled “Novel Design Considerations in the Development of a Photoplethysmography Ring” discloses a wearable health monitoring apparatus comprising a light source and a detector configured to receive transmitted and/or reflected light from a tissue sample, wherein the source and/or detector are incorporated into protrusions located within a ring-like structure. US Patent application 2017/0042477 A1 by Haverinen et al., entitled “Wearable electronic device and method for manufacturing thereof”, discloses a wearable electronic device which may be worn on the finger, operable to measure different physiological parameters, such as blood volume pulse, to determine a heart rate of the user. U.S. Pat. No. 10,281,953 B2 to von Badinski et al., entitled “Wearable Computing Device and Transmission Method” discloses a wearable computing device configured as a ring for being worn around the finger of a user, comprising sensor modules that enable the device to perform multiple functions to include a heart rate sensor and pulse oximetry. US Patent application publication 2016/0066827 A1 by Workman and Bomsta, entitled “Pulse Oximetry Ring”, discloses a wearable finger can provide a variety of biometrics (including heart rate, blood oxygen level, and skin temperature) and health measures (e.g., fall detection, sleep pattern recognition, and movement tracking). US Patent application 2010/0298677 A1 by Lu et al., entitled “Wireless ring-type physical detector”, discloses a ring-type physical detector that uses a light signal to detect the blood oxygen saturation, the heartbeat and continuous blood pressure. Lu et al. also teach that the ring further comprises an adjusting belt for changing the inner diameter of the ring. US Patent application publication 2020/0085360 A1 by Yuan and Zhou, entitled “Ring-type pulse oximeter”, discloses a ring-type pulse oximeter, comprising, in part, an elastic device, a photodiode, and at least one light emitting diode that are protrudingly disposed on an inner circumferential surface of ring body. When the ring is worn, the elastic device is pressed so that the photodiode and at least one light emitting diode fit with a finger, and light emitted by the light emitting diode is attenuated by the finger, then received by the photodiode and processed to calculate blood oxygen saturation. The ring-type pulse oximeter exerts a force on a portion of the finger, such that the finger maintains a tight fit to the photodiode and the light-emitting diode, thereby providing a comfortable wearing experience as well as adaptability to different finger shapes, and improving measurement accuracy.

U.S. Pat. No. 9,711,060 B1 to Lusted et al., entitled “Biometric sensor ring for continuous wear mobile data applications” discloses a biometric sensing ring worn on the finger for estimating the emotional state of a user. The ring is configured with a plurality of sensors for sensing electrodermal activity (EDA), photoplethysmograph (PPG), temperature, and acceleration. The invention derives emotion metrics from the data collected by the biometric sensing ring, which includes heart rate (HR), heart rate variability (HRV), and respiration rate based on HRV. The ring is configured for creating variable ring geometry to accommodate different sized fingers while offering comfortable fit for the user. Lusted et al., teach the sensors must be in stable contact with the skin in order to acquire optical EDA and PPG data.

As evidenced by the above review of relevant prior art, there has been significant innovation in determining various physiological parameters with wearable devices, in particular finger rings. However, the above prior art does not disclose the determination of hydration based on aortic valve opening and closing with a wearable device.

### SUMMARY OF INVENTION

Some embodiments of the present invention provide an apparatus for determining the hydration status of a user, comprising: (a) a ring, having an internal surface with an effective internal diameter, configured to be worn around a finger of the user; (b) an optical sensor system comprising (i) one or more optical emitters mounted with the ring such that light emitted by the one or more emitters is directed toward the finger and (ii) one or more detectors mounted with the ring such that the one or more detectors produce a detector signal representative of light reaching the detectors from one or more emitters after the light has interacted with tissue of the finger, configured to detect physiological signals indicative of opening and closing of the user's aortic valve; (c) a trigger system, configured to detect an event indicating a hydration measurement is to be initiated; (d) an optical sampling control system responsive to the trigger system configured to operate the one or more emitters and the one or more detectors at a first set of operational parameters; e) an analysis system responsive to the detector signal and configured to determine an interbeat time interval between successive openings of the user's aortic valve, and an ejection time interval between opening and closing of the user's aortic valve; (f) a hydration determination system configured to determine the hydration status of the user from the interbeat time interval and the ejection time interval; (g) a feedback system configured to provide feedback. Some embodiments further comprise a user input system configured to receive input from the user, and wherein the hydration determination system is configured to determine the hydration status of the user from the interbeat time interval and the ejection time interval and from the input. Some embodiments further comprise a posture determination system configured to determine the user's posture responsive to optical sensor system, the user input system, or a combination thereof, and wherein the hydration determination system is configured to determine the hydration status from the interbeat time interval, the ejection time interval, and the user's posture at the time the detector signal is produced. In some embodiments, the hydration determination system is configured to determine the hydration status from the interbeat time interval and the ejection time interval at a first posture, and from the interbeat time interval and the ejection time interval at a second posture.

In some embodiments, the analysis system is further configured to determine the suitability of the detector signal for hydration determination. Some embodiments further comprise a motion sensor system, and wherein the analysis is configured to determine the suitability of the detector signal responsive to the motion sensor system. In some embodiments, the optical sampling control system is configured to change the operational parameters responsive to the suitability of the detector signal determined by the analysis system. In some embodiments, the ring is configurable to assume a plurality of effective internal diameters such that, when the ring is configured to a first effective internal diameter, the venous transmural pressure in the tissue of the finger that has interacted with the light is less than zero and the arterial transmural pressure at diastole in the tissue of the finger that has interacted with the light is greater than zero. In some embodiments, the ring is configurable to assume a plurality of effective internal diameters such that the ring can be configured to a first effective internal diameter, producing a first set of transmural pressures in blood vessels in the tissue of the finger, and to a second effective internal diameter, producing a second set of transmural pressures in the blood vessels, where the pressures in the second set of transmural pressures are smaller than the pressures in the first set of transmural pressures. In some embodiments, the ring is configurable to either of two stable states wherein the first stable state the ring has a first effective internal diameter, and wherein the second stable state the ring has a second effective internal diameter distinct from the first effective internal diameter. In some embodiments, the ring has a mechanical bias that encourages the ring to the second stable state. In some embodiments, the second effective internal diameter is less than the first effective internal diameter.

In some embodiments, the trigger system comprises a sensor sensitive to a change in the effective internal diameter of the ring. In some embodiments, the trigger system is responsive to the optical sensor system, the user input system, or a combination thereof. Some embodiments further comprise a motion sensor system comprising an accelerometer, a gyroscope, or a combination thereof; and wherein the trigger system is responsive to the motion sensor system. In some embodiments, the ring comprises one of more compressive features that protrude from the inner surface of the ring, and wherein the effective internal diameter can be altered by the movement of the one of more compressive features. In some embodiments, the ring comprises one or more ring features, and wherein the effective internal diameter can be altered by movement of the one or more ring features along the longitudinal axis. In some embodiments, the ring has a reducible internal circumference. In some embodiments, the ring has ring features comprising protuberances on the inside of the ring whose configurations can be changed between first and second configurations, wherein the ring has a first effective internal diameter when the protuberances are at the first configuration and a second effective internal diameter, different from the first effective internal diameter, when the protuberances are at the second configuration.

In some embodiments, the user feedback system comprises one or more LEDs or haptic sensors mounted with the ring. In some embodiments, the user feedback system comprises an external device in communication with the ring, wherein the external device comprises a visible display. In some embodiments, the one or more optical emitters and the one or more detectors are mounted with the ring such that light reaching the detector comprises a majority of photons that have traveled through the tissue and interacted with tri-layered vessels. In some embodiments, an angle between an emitter and a detector, measured from the center of the ring, is greater than 15 degrees.

Some embodiments provide a method of determining the hydration status of a user, comprising: (a) providing a ring configured for wearing around a finger of the user wherein the ring comprises one or more optical emitters mounted with the ring such that light emitted by the one or more emitters is directed toward the finger, and one or more detectors mounted with the ring such that the one or more detectors produce a signal representative of light reaching the one or more detectors from one or more emitters after the light has interacted with tissue of the finger; (b) triggering a hydration measurement by one or more of a user-based, time-based, or signal-based event; and then (c) operating the one or more emitters and the one or more detectors using a first set of operational parameters and acquiring a signal from the detector representative of light interaction with a sampling region of the finger; d) determining from the detector signal the interbeat time interval between successive openings of the user's aortic valve and the ejection time interval between opening and closing of the user's aortic valve; (e) determining the hydration status of the user from the interbeat time interval and the ejection time interval. Some embodiments further comprise prior to step (c) establishing a first set of transmural pressures in the blood vessels in the sampling region, such that the venous transmural pressure in the sampling region is less than zero and the arterial transmural pressure at diastole in the sampling region is greater than zero.

Some embodiments further comprise determining a metric indicative of the suitability of the detector signal for determining hydration status. Some embodiments further comprise determining if the metric is within predetermined bounds, and, if not, repeating step (c) using a second set of operational parameters, different from the first, before performing step (d). Some embodiments further comprise determining if the metric is within predetermined bounds, and, if not, establishing a second transmural pressure, different from the first transmural pressure, and repeating step (c) before performing step (d). Some embodiments further comprise determining if the metric is within predetermined bounds, and, if not, establishing a second transmural pressure, different from the first transmural pressure, and repeating step (c) using a second set of operating parameters, different from the first, before performing step (d).

In some embodiments, step (c) is repeated a plurality of times, each time using a different set of operational parameters and together producing a plurality of detector signals, and wherein step (d) comprises determining the hydration status from the plurality of detector signals. In some embodiments, step (c) is repeated a plurality of times, each time using a different set of operational parameters and together producing a plurality of detector signals, and further comprising determining a metric indicative of the suitability of the detector signal for determining hydration status for each of the detector signals; and wherein step (d) comprises determining the hydration status from the plurality of detector signals weighted by the metric for each of the plurality of detector signals. In some embodiments, detector signals corresponding to a metric outside a predetermined range are weighted at zero in step (d). In some embodiments, step (c) is performed while the user is in a first posture to produce a first detector signal and while the user is in a second posture to produce a second detector signal; and wherein step (d) comprises determining the hydration status from first and second detector signals.

In some embodiments, the ring has an adjustable effective internal diameter, and wherein establishing a transmural pressure comprises establishing the effective internal diameter of the ring. In some embodiments, establishing a transmural pressure comprises positioning the hand on which the ring is worn to a predetermined elevation relative to the heart. In some embodiments, establishing a transmural pressure comprises moving the ring to a different finger region. In some embodiments, establishing a transmural pressure comprises pushing a portion of the ring toward the finger. In some embodiments, step (e) comprises determining the hydration status of the user from the interbeat time interval and the ejection time interval and the user's posture when the detector signal was produced.

Some embodiments further comprise determining the posture of the user from one or more of an accelerometer mounted with the ring, a gyroscope mounted with the ring, or optical sensors mounted with the ring. Some embodiments further comprise accepting from the user an indication of the user's posture.

In some embodiments, step (e) comprises determining the hydration status of the user from the interbeat time interval and the ejection time interval and one or more of the user's age, gender, weight, or height at the time the detector signal was produced. In some embodiments, step (c) is performed two times, the first time using operational parameters that establish a transmission dominant sampling, and the second time using operational parameters that establish a reflectance dominant sampling; and determining which detector signal has the strongest aortic closure signal, and using that detector signal in step (d). Some embodiments further comprise displaying the hydration status on a device separate from and in communication with the ring. Some embodiments further comprise providing visual, aural, or haptic feedback to the user using the ring. Some embodiments further comprise providing a plurality of rings distinct in appearance from each other, and providing feedback to the user if a battery powering a first ring is low, such that the user can use a second ring.

Some embodiments provide a method for determining the hydration status of a user, comprising: (a) acquiring a signal from a wearable sensor nonobtrusive to the activities of daily life, configured to detect changes in blood volume in a measurement region of the user, which changes are indicative of opening and closing of the user's aortic valve, while the user is in one or more distinct postures; (b) using a hydration determination model to determine the hydration status of the user from the signal determined at one or more postures; (c) communicating the hydration status to the user.

In some embodiments, step (a) further comprises determining the posture and the maintenance of the posture by the user during the acquisition of the signal. In some embodiments, step (b) comprises (b1) determining an interbeat time interval between successive aortic valve openings; (b2) determining an ejection time interval between aortic valve opening and aortic valve closing; and (b3) determining the hydration status from the interbeat time interval, and the ejection time interval determined at one or more postures. In some embodiments, step (b) comprises (b1) determining an interbeat time interval between successive aortic valve openings; (b2) determining an ejection time interval between aortic valve opening and aortic valve closing; and (b3) determining the hydration status from the interbeat time interval, the ejection time interval, and the posture determined at one or more postures. In some embodiments, the sensor is worn around a finger, wrist or upper arm of the user. Some embodiments further comprise establishing a transmural pressure in the blood vessels contained in measurement region, such that the transmural pressure in veins in the region is less than zero and the transmural pressure in arteries in the region at diastole is greater than zero.

The invention provides a situationally aware, adaptive hydration monitoring system. Situation awareness is built upon two complementary awareness mechanisms, each capable of acting independently or in concert, resulting in increased measurement cadence. First, a situational-awareness subsystem continuously evaluates real-time physiological signals (e.g., heart-rate deviation, skin temperature, respiratory rate, activity level) and environmental factors (e.g., ambient temperature, humidity, geolocation) to determine if the user will likely experience a significant change in hydration. The second system, a predictive-assessment subsystem, computes a rate of change (e.g., derivative) of successive hydration measurements to forecast future fluid status and identify when the subject may leave a prescribed hydration range. Whenever either subsystem exceeds its configurable threshold, a cadence control system alters the measurement frequency to ensure clinical fidelity for effective hydration management.

The circumstances that drive hydration changes, such as physical motion, diminished pulse size, elevated heart rate, surface perspiration, and bright sunlight, often compromise plethysmographic signal quality. To guard against poor plethysmographic data, a measurement suitability system evaluates the incoming pulses. When quality drops such that accurate left ventricular ejection time and heart rate cannot be determined, an operational-control system modifies operational parameters in real time. Typical operational changes include raising the sampling frequency, changing transmural pressure, adjusting the optical acquisition profile, and opportunistically timing measurements for moments that naturally favor stable signal capture. This closed-loop strategy ensures that the system maintains reliable data despite the very factors that perturb hydration in the first place.

The situational-awareness, predictive-assessment, measurement-suitability, and operational-control subsystems create a closed-loop architecture that delivers high-resolution hydration tracking only when needed, conserving battery life during physiologically quiet periods and stepping up performance when conditions demand. When either awareness layer calls for improved temporal detail, the cadence control system temporarily increases measurement frequency; conversely, when the measurement suitability system detects degraded signal quality, the operational-control system retunes acquisition conditions. By orchestrating both how often and how well data are collected, the invention ensures that every hydration estimate based on plethysmographic signals meets predefined fidelity thresholds, even under the motion, perspiration, and lighting challenges that accompany real-world use.

### BRIEF DESCRIPTION OF DRAWINGS

FIG. 1 illustrates the effects mental and physical effects of dehydration.

FIG. 2 presents the different water compartments in the human body.

FIG. 3 is an error sensitivity comparison table for different approaches to hydration assessment.

FIG. 4 shows the relationship between several measurable signals and cardiac function.

FIG. 5 represents a Sagawa pressure-volume loop.

FIG. 6 demonstrates the relationship between left ventricular volume and pressure.

FIG. 7 illustrates pressure-volume curves under conditions of changing hydration.

FIG. 8, comprising FIGS. 8A, 8B, 8C, 8D, 8E, and 8F, shows example measurement locations of the invention.

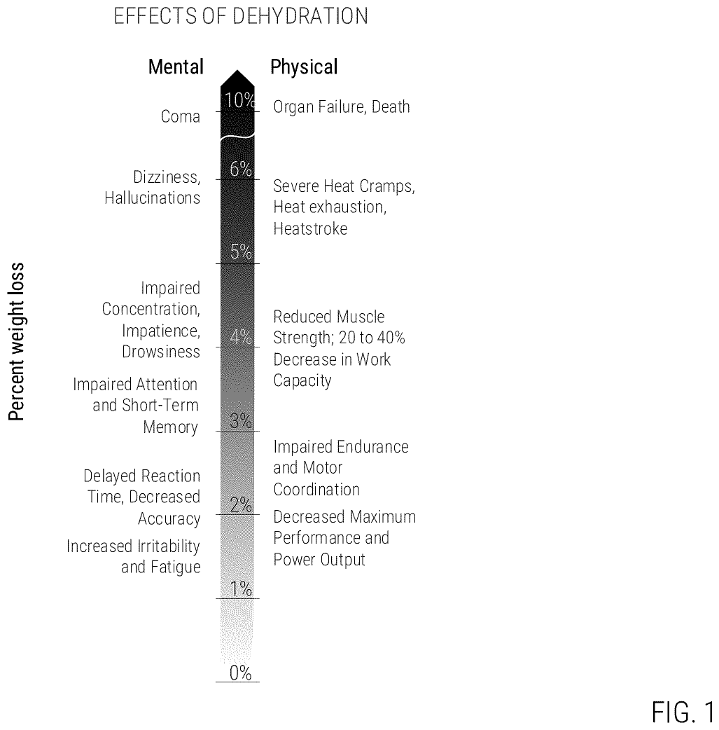

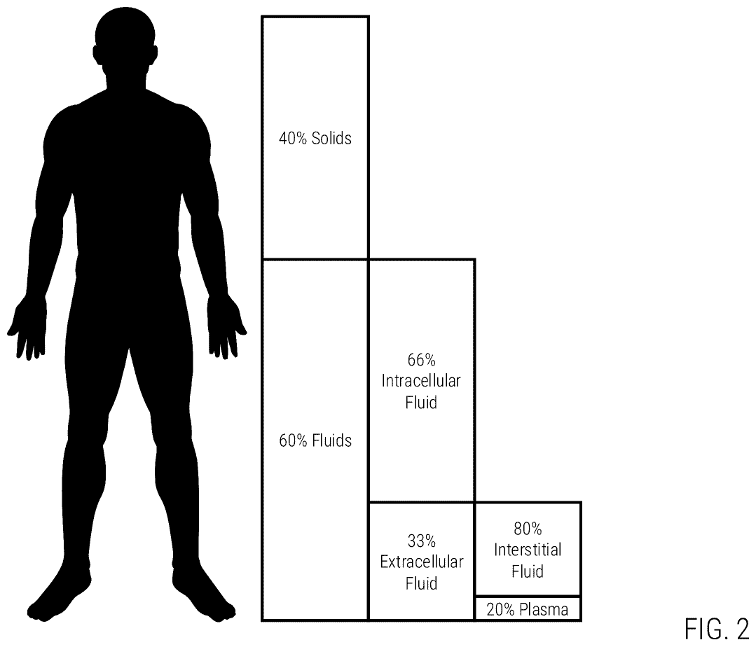

FIG. 1, comprising FIGS. 9A, 9B, and 9C, shows the influence of sampling resolution on determination of aortic valve closure.

FIG. 10 shows the effect of decreasing transmural pressure on pulse size.

FIG. 11 is a second example of the effect of decreasing transmural pressure on pulse size.

FIG. 12 shows a typical relationship between heart rate and ejection time.

FIG. 13 is schematic of heart rate vs ejection time at two hydration levels.

FIG. 14 is an illustration showing the impact of body position on venous return.

FIG. 15 shows the relationship between ejection time and body posture.

FIG. 16 shows heart rate and ejection time relationships at different body postures.

FIG. 17 illustrates a set of the inputs that can be used for hydration determination.

FIG. 18 illustrates an alternative set of the inputs for hydration determination.

FIG. 19 shows an illustrative embodiment of the apparatus used to measure the aortic valve time series.

FIG. 20 presents an example of how the systems of the illustrative embodiment can interact.

FIG. 21 defines a coordinate system for a finger or other body member.

FIG. 22, comprising FIGS. 22A, 22B, 22C, 22D, and 22E, defines the effective internal diameter for a ring-type device.

FIG. 23, comprising FIGS. 23A, 23B, 23C, 23D, and 23E, shows example mechanisms to change the effective internal diameter of a ring.

FIG. 24, comprising FIGS. 24A, 24B, and 24C, shows example mechanisms to change the effective internal diameter of a ring.

FIG. 25, comprising FIGS. 25A, 25B, and 25C, shows example mechanisms to change the effective internal diameter of a ring.

FIG. 26 shows example mechanisms to change the effective internal diameter of a ring.

FIG. 27 shows example mechanisms to change the effective internal diameter of a ring.

FIG. 28, comprising FIGS. 28A and 28B, shows example mechanisms to change the effective internal diameter of a ring.

FIG. 29, comprising FIGS. 29A and 29B, shows example mechanisms to change the effective internal diameter of a ring.

FIG. 30 shows example mechanisms to change the effective internal diameter of a ring.

FIG. 31 presents an example of the steps to determine hydration status.

FIG. 32 presents an example process for triggering a hydration measurement and providing feedback.

FIG. 33 presents an example process for determining hydration status with the possible variation of operational parameters and transmural pressure.

FIG. 34, comprising FIGS. 34A, 34B, 34C, 34D, and 34E, illustrates measurement results during exercise-induced hypertonic dehydration.

FIG. 35, comprising FIGS. 35A, 35B, 35C, 35D, and 35E, illustrates measurement results during exercise with and without fluid replenishment.

FIG. 36 illustrates heart rate and ejection time during simulated isotonic dehydration.

FIG. 37, compriding FIGS. 37A, 37B, and 37C, shows heart rate, mean arterial pressure, and ejection time during simulated changes in hydration.

FIG. 38 illustrates heart rate and ejection time acquired during exercise.

FIG. 39, comprising FIGS. 39A and 39B, shows measurement results using positional changes in the hydration assessment.

FIG. 40, comprising FIGS. 40A and 40B, shows examples of pairs of rings that may be provided to a user.

FIG. 41 shows operational flows chart associated with operation of the system

FIG. 42 shows an example embodiment of a temple based hydration sensor

### INDUSTRIAL APPLICABILITY AND MODES OF CARRYING OUT THE INVENTION

### Definitions.

For the purposes of this invention, hydration or dehydration are defined broadly as a measure of the amount of water present in the body. Changes in hydration status occur when water intake is inconsistent with changes in free water lost due to normal physiologic processes, including breathing, urination, and perspiration, or other causes, including diarrhea and vomiting. Total body water (TBH2O) represents about 45-60% of body weight depending on age, gender, and race. TBH2O is further divided into an intracellular fluid compartment (ICF; about 60% of total body water) and an extracellular fluid compartment (ECF; about 40% of total body water), which are proportional to the ratio of osmotically-active intracellular K+ to extracellular Na+. During normal physiology these compartments are dynamically equilibrating to maintain whole-body fluid balance.

For the purposes of this invention, dehydration includes hypertonic, isotonic, and hypotonic dehydration. Hypertonic dehydration occurs when more water is lost from the body than salt, increasing blood osmolality. Increased sweat rate is a common cause of hypertonic dehydration. Isotonic or hypotonic dehydration occur when the amount of water lost is equal to or less than the amount of salt lost, respectively. Isotonic dehydration is commonly caused by diarrhea or blood loss.

As used herein, effective circulating volume, vascular volume, and blood volume are interchangeable terms. The effective circulating volume refers to that part of the extracellular fluid compartment that is within the vascular space and is effectively perfusing the tissues. Circulating volume refers to the total amount of fluid circulating within the arteries, capillaries, veins, venules, and chambers of the heart at a given time that is available to the heart for pumping.

As used herein, the time course of aortic value opening and closing refers to any data representation that contains the relationship between the status of the aortic value and some measurement of time.

As used herein, the ejection time (ET) is the time interval between the aortic valve opening (AVO) and the aortic valve closure (AVC). Blood is ejected from the left ventricle during this interval.

As used herein, the interbeat interval (IBI) refers to the time interval between similar points in the cardiac cycle. For example, the IBI can be defined as the time between aortic valve openings (AVOs) in successive cycles.

As used herein, the terms “body posture”, “body position”, or “body pose” refer to the different physical configurations that the human body can assume. The most common body postures include supine (lying on the back), seated, and standing positions.

As used herein, “positional changes”, “posture changes”, or “changes in pose” are terms that refer to any process that alters body position in a manner that changes the venous return to the heart. For example, one simple way to manipulate venous return via positional changes is to move between supine, seated, and standing positions.

The term “determination model” or “determination system” as used herein is broadly defined as any process that takes defined inputs and applies calculations or a designated set of steps to determine a desired output. Determination models include many classes of models but can be broadly broken into “prediction models” and “matching models”. Prediction models are constructed by determining the relationship between data or data features and desired output; once the relationship is determined the model can be applied to novel data with no reliance on the training or reference data. These models are distinct from matching models which rely on pre-existing library of training or reference data. A matching model determines the proximity of novel data to reference data to produce the desired output. Examples of prediction models include regression models, where features are mapped to outputs through linear or non-linear relationships, as well as some machine learning models, in which more complex data representations are mapped to the desired output. In these approaches, often referred to as “deep learning models”, the useful features and representations are essentially learned by the model in training, along with the function that maps the inputs to the desired outputs. Because the relationship between input and outputs is often quite complex (involving thousands of weights in multiple hierarchical layers) the engineer or architect of the model might be completely unaware of the features or information that the model has extracted, or how and why that information is combined to form the output. In some embodiments of this invention, the determination model can use as inputs features extracted from a data representation that contains the relationship between the status of the aortic value and some measurement of time. In other embodiments, the determination model can use as inputs a raw or conditioned data representation that contains the relationship between the status of the aortic value and some measurement of time. A determination model can also include additional inputs, such as body position or information about the user. The output of a given determination model is the desired parameter, such as hydration status.

Transmural pressure is a general term that describes the pressure across the wall of a vessel (transmural literally means “across the wall”). A flexible container expands if there is a positive transmural pressure (pressure greater inside than outside the object) and contracts with a negative transmural pressure. A positive transmural pressure is sometimes referred to as a “distending” pressure.

Speckle plethysmograph (SPG), as used herein, is a noninvasive optical measurement system that measures blood flow in the body. The system uses a laser, coherent light sources, or other light source to illuminate the skin and tissue, and then analyzes the scattered light patterns, or speckles, which are produced. The system can operate in reflection sampling mode and transmission and transmission sampling mode. The system can be used to measure blood flow in various parts of the body, such as the hand, finger, wrist, foot, or brain, and can provide important information about the function of the circulatory system and the health of tissues and organs. A speckle sensor system creates a plethysmogram representing changes in blood flow over the cardiac cycle and has a signal that is related to the cardiac cycle and contains aortic value opening and closure information.

Photo plethysmograph (PPG), as used herein, is an optical measurement system that measures changes in blood volume using changes in light absorption and can be used to measure blood volume in a transmission sampling mode and reflection sampling mode. The measured signals, a plethysmogram, can be used to calculate both physiological and cardiometric parameters for both physiological assessments and the determination of cardiac fitness. A PPG system creates a photo plethysmogram representing changes in blood volume over the cardiac cycle and has a signal that is related to the cardiac cycle and contains aortic value opening and closure information.

Radar plethysmograph (RPG) is a noninvasive millimeter-wave, radar-based device for the accurate measurement of arterial pulse waveforms. Radar plethysmography can be utilized at any location on the body where a pulse creates a detectable movement of the skin or tissue. A common location is to use the system as a wrist-worn device that positions the radar near the radial artery without touching the skin, allowing for interrogation of the pulse at close range without perturbing the pulse waveform. The resulting information has a signal that is related to the cardiac cycle and contains aortic value opening and closure information.

Plethysmographic data or signals, as used herein, denotes any raw or processed time-series signal that captures moment-to-moment changes in tissue volume or blood flow—whether obtained optically, electrically, or mechanically. These data can include the full waveform as well as derived parameters such as amplitude, area under the curve, rise time, and systolic time intervals that characterize cardiovascular dynamics and related physiological states.

Plethysmographic analysis system, as used herein, denotes an integrated hardware-and-software subsystem that acquires plethysmographic data or signals—i.e. any raw or processed time-series waveform capturing moment-to-moment changes in tissue volume, or blood flow—and applies filtering, signal transformation, noise-cancellation, feature detection, and analytic algorithms (such as probabilistic or prediction models) to extract key parameters (e.g., ejection time, interbeat interval, systolic time intervals) and derive physiological or cardiometric metrics.

Prediction model, as used herein, means any algorithmic, mathematical, heuristic, physics-based, or machine-learned process—implemented in hardware, firmware, software, or any combination thereof—that receives measured, derived, or assumed inputs and returns an estimated, inferred, classified, or forecast output. The term deliberately embraces the full spectrum of analytic techniques, from explicit parametric equations and compartment models to non-parametric or distribution-free methods, rule-based and fuzzy-logic systems, physics-informed simulations, and contemporary machine-learning architectures such as decision trees, ensemble methods, neural networks, transformers, and adaptive or online-learning variants. It applies whether the computation is deterministic or stochastic, supervised or unsupervised, executed in real time or batch mode, and whether it runs locally on-device, at the network edge, in the cloud, or in any distributed or federated environment.

Hydration Determination System, as used herein, denotes an integrated hardware-and-software platform that acquires plethysmographic data or signals, optionally derives plethysmographic parameters from those signals, and then applies a prediction model to those data to produce a quantitative or qualitative indication of the user's hydration status. The definition encompasses all practical embodiments, wearable, phone based, or otherwise, irrespective of sensor modality or location, signal-processing technique, model architecture, power management scheme, or deployment location, provided the system creates a predictive inference of hydration.

Wearable housing, as used herein, denotes any enclosure, band, shell, or chassis configured to be worn on the body (e.g., finger ring, wristband, eyeglass temple, adhesive patch) and to mechanically support the optical sensor system, contextual-sensor suite, electronics, and power source required for hydration monitoring.

Optical sensors, as used herein, refers to any optically based system that can be used to capture signals related to changes in blood volume, flow, or pressure in a measurement region of the individual, which changes are indicative of cardiac function.

Optical sensor system, as used herein, comprises at least one light emitter and at least one photodetector arranged to deliver photons into tissue and receive a resulting signal so that aortic-valve opening and closing events can be resolved.

The term “signal” as used herein includes any means of transmitting information such as a measurement, including without limitation an analog electrical waveform or digital representation thereof, e.g., that which is collected or transmitted by a biological or physiological sensor, such as a PPG.

The term “noninvasive” refers to a method or apparatus that does not create a break in the skin and makes no contact with an internal body cavity beyond a natural body orifice. PPG and EKG sensors are examples of noninvasive sensors that can make measurements without breaking the skin. Likewise, PPG is an example of noninvasive sampling, wherein measurements are acquired optically from the skin surface without introducing instruments into the body.

As used herein, the term “emitter” describes any device emitting electromagnetic radiation. One example of an emitter is a light emitting diode (LED).

The terms “photodetector”, “optical detector”, or simply “detector”, refer to any device that detects or responds to incident light by using the electrical effect of individual photons.

As used herein, the term “tri-layered vessels” refers to blood vessels comprised of three layers: the tunica intima, the tunica media, and the tunica adventitia. Tri-layered vessels include arteries, arterioles, venules, and veins, but do not include capillaries, which are comprised of a single layer of endothelial cells.

Transmission dominant sampling refers to optical sampling of the tissue where the majority of photons penetrate and travel through the tissue, interacting with (i.e., reflected by, scattered by, or absorbed by) tri-layered vessels.

Reflection dominant sampling refers to optical sampling of the tissue where the majority of photons do not penetrate deeply into the tissue and primarily interact with (i.e., are reflected by, scattered by, or absorbed by) vessels in the capillary bed.

As used herein, the term “deformable” broadly describes an object that changes its shape or volume while being acted upon by an external force. The process of deformation can occur within a single deformable component, for example, one with elastic material properties that may stretch or bend, or through the respective movement of rigid components, as seen in, for example, telescopic expansion and hinges.

As used herein, “user-based” or “user-initiated” events or triggers broadly refer to a process in which a hydration determination is performed responsive to an action of the user. The user action may include, but is not limited to, a gesture, specified motion, application of force, button press, vocal expression, or communication through a connected device representing a volitional choice on the part of the user to obtain a hydration assessment.

As used herein, the terms “activities of daily life” or “activities of daily living” refer to the routine activities people do every day in normal life. Minimally, these activities include eating, bathing, getting dressed, using the toilet, and getting in and out of bed.

As used herein, the “effective internal diameter” of a ring is defined as the diameter of the largest possible circle that can be inscribed in the ring when viewed as a longitudinal projection.

For the purposes of this invention, the “sampling” is defined as the acquisition of physiological data from a user. The related terms, “sampling site”, “sampling region”, and “sampling location”, refer to a region of a user where sampling is performed. When physiological data are acquired using an optical system, these terms refer to the region where light interacts with the tissue.

Standard-fidelity sampling, as used herein, is defined as a class of optical-sensor operational parameters that are adequate to detect prominent cardio-physiologic events-most notably aortic-valve opening-thereby enabling heart-rate or inter-beat-interval determination, but that do not ensure the reliable resolution of lower-amplitude features such as aortic-valve closure. Exemplary parameter ranges include a sampling frequency≤100 Hz, a single emitter, and one photodetector. Standard-fidelity sampling is a default “wear-mode” configuration chosen for minimal power consumption and user comfort, and it serves as a baseline against which higher-performance modes are defined

Burst sampling, as used herein, refers to a conventional engineering term that describes a temporary, high-density acquisition period inserted between longer intervals of lower-density sampling. The device escalates from Standard-fidelity sampling to a short-lived, more intensive mode whenever a Hydration-Change Period is detected or forecast. Burst sampling defines a class of operational parameters that supports the concurrent detection of aortic-valve opening and aortic-valve closing events. Burst sampling can comprise any combination of (i) increased sampling rate (e.g., ≥100 Hz), (ii) elevated light-source drive current, (iii) extended or adaptive detector-integration time, (iv) expanded sample averaging or stacking, and/or (v) activation of additional wavelengths, emitters, or photodetectors. Burst sampling may be associate with changes in transmural pressure. These parameter increases are defined relative to Standard-fidelity sampling and are invoked when higher temporal or amplitude resolution is required for hydration assessment. Burst sampling can also be referred to as high-fidelity sampling.

Measurement cadence or measurement frequency, as used herein, defines the number of hydration measurements made per unit of time.

Sampling frequency, as used herein, means the rate at which the optical-sensor subsystem acquires individual plethysmographic data points, expressed in samples per second (Hz). A sampling frequency of 100 Hz therefore corresponds to 100 discrete optical acquisitions each second, irrespective of how often full hydration determinations are performed.

Situational awareness, as used herein, describes the continuous recognition and evaluation of both internal physiological conditions and external environmental factors—and their combined influence on hydration risk—so that the device can automatically adjust its sensing cadence and operating parameters in real time.

Probability of Hydration Change System, as used herein, is the combination of hardware, firmware, and software that uses an algorithm or Bayesian or machine-learned estimator that fuses contextual inputs to output a hydration-change probability and optional rate, and may execute on an on-device micro-controller, companion application processor, smartphone or cloud service.

A probability score, probability of hydration change or a hydration-change probability score, as used herein means machine, software, or hardware-generated indicator, numeric or non-numeric, that represents the likelihood that a user's hydration status will cross, or has crossed, one or more predefined or adaptively determined limits within a specified time horizon. It includes any signal, value, or representation conveying information about the chance, risk, or likelihood that a user's hydration state will meet, exceed, or satisfy at least one hydration-related criterion within a reference interval.

Contextual parameters, as used herein, means the real-time combination of internal thermoregulatory signals (e.g., heart-rate deviation, skin temperature, respiratory rate, activity level) and external environmental factors (e.g., ambient temperature, humidity, solar radiation, or geolocation-derived weather data), which together influence the computation of hydration-change probability and control adaptive measurement parameters.

Thermoregulatory signals, as used herein, are measurable parameters whose magnitude or rate of change correlates with the user's endogenous heat production or the body's capacity to dissipate that heat. The parameters include, but are not limited to, heart rate deviation from baseline, skin temperature, respiratory rate, heart rate variability, activity level, motion identification, and sympathetic tone.

Burst-sampling controller, as used herein, is control logic that compares the hydration-change probability, hydration adequacy trigger and baseline cadence and, when threshold criteria are met, commands the optical sensor system to enter burst sampling (≥100 Hz and/or enhanced operational parameters).

Operational parameters, as used herein, are the adjustable data acquisition settings that shape the physical and electronic conditions under which plethysmographic—or other physiologic—signals are captured. They fall into three broad categories: (1) temporal settings, which include sampling frequency, integration or averaging window, duty-cycle scheduling, and any sleep/awake timing of the electronics; (2) mechanical settings, such as sensor-to-skin transmural pressure, emitter-detector spacing, and other forces that influence optical coupling or tissue perfusion; and (3) photonic or electronic settings, collectively referred to as the optical acquisition profile, encompassing emitter drive current, number and wavelength of emitters, duty-cycle sequencing, detector count and type, detector gain, and detection bandwidth. Whenever the measurement suitability system flags inadequate signal quality, the operational-control system can return anyone, or any combination, of these parameters, thereby restoring the signal-to-noise ratio required for reliable hydration assessment.

Motion-Scenario-Opportunity Sampling (MSO Sampling), as used herein, is the device's opportunistic strategy for capturing plethysmographic data at moments most likely to yield high-quality signals with minimal motion artefacts. The strategy relies on three real-time filters working in concert: (i) Motion, which detects brief intervals of minimal or absent movement; (ii) Scenario, which recognizes contextual cues—such as activity type, posture, or environment-that suggest forthcoming periods of lower motion or improved signal quality; and (iii) Opportunity, which triggers a standard measurement-or a sequence of short measurement windows that can be aggregated—when the combined Motion and Scenario filters predict a high probability of obtaining data suitable for hydration assessment. By concentrating acquisition within these windows, MSO Sampling maximizes the probability of acquiring high signal-to-noise ratio data without forcing the sensor to remain in a high-energy state when the likelihood of success is low.

Optical acquisition profile, as used herein, is a collection of configurable parameters that govern emission and detection of optical signals within the sensor system. Without limitation, the profile may specify one or more of: emitter drive current, number of emitters, emitter wavelength or spectral band, emitter duty cycle, detector count, detector type, detector gain, detector sampling frequency, optical path geometry, and any timing, sequencing, or multiplexing scheme applied to the foregoing elements.

Operational-control system, as used herein, the operational-control system is the combination of hardware, firmware, and software that executes real-time adjustments to the sensor's operational parameters whenever prompted by data-quality requirements or power-management policies. It receives suitability flags (or graded confidence scores) from the measurement-suitability system and responds in two complementary ways. First, it retunes operational parameters—such as sampling frequency, sensor-to-skin transmural pressure, or elements of the optical-acquisition profile—to restore or maintain the signal-to-noise ratio needed for accurate hydration assessment. Second, it orchestrates MSO Sampling within each measurement cadence, opening brief, context-appropriate windows for data capture whenever motion subsides and scenario cues indicate a favorable opportunity. By coupling adaptive parameter control with MSO Sampling, the operational-control system delivers reliable hydration assessment across diverse activities while minimizing unnecessary energy expenditure.

Cadence control system, as used herein, is a set of hardware, firmware, and/or software elements that dynamically determine and implement the temporal spacing of hydration-related measurements. Without limitation, the Cadence control system (i) operates with a baseline measurement interval, (ii) adjusts that interval upward or downward in response to inputs from the situational-awareness subsystem and predictive-assessment subsystem. In some embodiments, the Cadence control system is configured to conserve power during stable hydration periods yet increases measurement frequency whenever required to meet the clinical-fidelity criteria.

Measurement suitability system, as used herein, is the hardware, firmware, and/or software logic that processes and evaluates the plethysmographic (or otherwise physiologic) data and decides whether that data is the characteristics needed for hydration analysis. The plethysmographic analysis system can be contained inside the measurement suitability system, and process the data for determine of heart rate, LVET and other cardiometric parameters and Without limitation, the measurement suitability system may: (i) compute quantitative quality metrics such as signal-to-noise ratio, pulse-shape similarity, motion-artifact indices, or confidence scores for derived features (e.g., heart rate and left-ventricular ejection time); (ii) compare those metrics against one or more predefined or adaptive thresholds; and (iii) output a binary or graded “suitability flag.” When the flag indicates inadequate quality, the system communicates with the operational-control system to trigger corrective actions, as further described herein.

Clinical fidelity, as used herein, is a context-dependent concept whose threshold will vary depending upon the patient population, the clinical environment, the patient's health condition and comorbidities, and the desired clinical objectives. The work of Sawka et al. states: “The goal of drinking during exercise is to prevent excessive (>2% body-weight loss) dehydration and excessive changes in electrolyte balance to avert compromised performance.” See Sawka M. N. et al., “Exercise and Fluid Replacement,” Med. Sci. Sports Exerc. 39(2): 377-390 (2007). Hypohydration of ≥2% body mass is a common boundary for dehydration determination and has been shown in many studies to define a boundary of impaired endurance performance and mental capacity across a range of exercise modalities and durations. This easy-to-measure guideline of 2% loss of body mass ignores the wide inter-individual spread in baseline total-body water (TBW), a figure that can range from ˜45% of weight in older, adipose individuals to ˜65% in lean, muscular adults. Because fat tissue contains far less water than muscle, two people who both lose 2% of their scale weight may experience markedly different thermoregulatory and cardiac stress. For this reason, the present invention casts clinical fidelity in terms of percentage change in TBW, a metric that normalizes for body-composition variance. Using the 2% body-mass benchmark and an average adult TBW of roughly 55% of weight, the corresponding “action point” is about 4% TBW depletion. To guarantee ample warning before that point is reached, the system sets two quantitative fidelity targets: (i) measurement accuracy within ±1.0% TBW, providing a four-fold safety margin relative to the 4% action threshold; and (ii) detection of cumulative changes as small as 0.5% TBW, optical sensor system yielding an eight-fold margin that enables timely clinical intervention even under rapid fluid shifts. These limits define one rationally based objective standard for “clinical fidelity.”

### Approaches to Wearable Hydration Determination

Conventional Approaches. Multiple groups have sought to create hydration measurement systems that are noninvasive and wearable. The majority of prior attempts have sought to measure the concentration of water within the skin or blood, and typically fall into one of the following approaches: (1) spectroscopic determination of constituent concentrations in the blood or tissue, (2) impedance-based determination of body water content, (3) pulse size or perfusion analysis, or (4) chemometric-based assessment of sweat. These methods and their challenges are discussed briefly.

Absorbance spectroscopy refers broadly to spectroscopic techniques that measure the absorption of radiation, as a function of frequency or wavelength, due to its interaction with a sample. Absorption spectroscopy is employed as an analytical chemistry tool to determine the presence of a substance in a sample and, in many cases, to quantify the amount of the substance present. In practice, absorbance measurements are challenging to implement due to instrumentation drift, the use of multiple wavelengths, instrument drift, pathlength differences, and tissue sampling errors. The degree of absorbance is determined by the light interaction with all materials located between the source and detector. The water content in the sweat on the surface of the skin absorbs at the same level as the same amount of water located in the skin, producing a significant error in methods relying on absorption. Additionally, changes in the physical relationship or interface between the tissue and the optical measurement system that change the measured absorbance represent problematic error sources. Such changes can easily occur during typical movement of the body. Absorbance measurements are highly sensitive to changes in the tissue-optical instrument interface, and skin surface contamination (e.g., by sweat or other substances).

US Patent application publication 2020/0000345 A1 by Connor, entitled “Wearable Ring of Optical Biometric Sensors”, is an example of an invention that tries to address the technological challenges of spectroscopic measurements of biometric parameters, to include hydration. Connor describes a wearable ring of sensors comprising an arcuate array of light emitters and receivers configured to collectively span at least half of the circumference of the finger, wrist or arm wearing the ring. The location, emission angle, distance, and pressure of the emitters can be adjusted such that the emitters remain in close optical communication with the surface of the finger, wrist, or arm even if the device shifts and/or rotates.

Spectroscopic assessment of hydration based on optically-determined hemoconcentration has also been proposed. The concept is based on the fact that as hydration changes, the number of red blood cells in the vascular system will remain roughly constant but the volume of fluid in the vascular compartment decreases. The result is mild hemoconcentration that occurs with dehydration. Most efforts have pursued analytical methods that isolate the signal to the arterial pulse. US Patent application publication 2015/0148623 A1 by Benaron, entitled “Hydration Monitoring Sensor and Method for Cell Phones, Smart Watches, Occupancy Sensors, and Wearables”, is an example of an invention for hydration monitoring with wearables and other devices that uses a spectroscopic approach. Benaron discloses estimating hydration by determining a measure of water content, said measure of water content at least in part based on a function of a concentration of components of the bloodstream or tissue of the subject over time determined using spectral analysis of the detected light.

Bioelectrical impedance analysis (BIA) is a commonly used method for estimating body composition, in particular body fat and muscle mass. In BIA, a weak electric current flows through the body and the voltage is measured in order to calculate impedance (resistance) of the body. BIA determines the electrical impedance, or opposition to the flow of an electric current, through body tissues which can then be used to estimate total body water (TBW), which can be used to estimate fat-free body mass and, by difference with body weight, body fat. Dehydration is a recognized factor affecting BIA measurements because it causes an increase in the body's electrical resistance. Thus, under the assumption of constant muscle mass, BIA can be used to determine a change in hydration, expressed as total body water. In a typical use-case the measurement process requires that four electrodes be attached to the body, typically attached to hands and feet. BIA is capable of estimating total body water with good accuracy in healthy subjects. However, the biophysical principles of BIA limits accuracy and applicability for hydration assessment. This is well described by O′Brien et al., who write, “while BIA can reliably estimate total body water and body density in euhydrated individuals under standardized clinical conditions, changes in fluid and electrolyte content can independently alter bioimpedance measurements. Because hydration changes typically involve concomitant changes in fluid and electrolyte content, the interpretation of a change in bioimpedance will often be confounded.” O'Brien, C., Young, A. J., & Sawka, M. N. (2002). Bioelectrical impedance to estimate changes in hydration status. International Journal of Sports Medicine, 23(05), 361-366. Thus, because BIA is dependent on both water and electrolyte concentrations, the type of hydration (e.g., isotonic or hypertonic) will have a significant impact on the assessment of hydration.

US Patent application publication 2016/0338639 A1 by Myers et al., entitled “Personal Hydration Monitor”, is an example of an invention for a hydration sensor in a wearable device based upon impedance. Myers et al. disclose a wearable hydration monitor comprising a flexible electrode on a flexible substrate configured to measure the level of hydration of an individual using a skin impedance measurement obtained by the electrode. US Patent application publication 2015/0182164 A1 by Utter, entitled “Wearable Ring for Sleep Monitoring”, is a second example of an invention of that proposes to use bioimpedance and other variety of other sensors to detect dehydration in a flexible and wearable ring. Utter discloses the potential use of a plurality of biometric sensors selected from the group consisting of a heart rate sensor, a respiration sensor, a temperature sensor, a skin conductance sensor, a skin conductance response sensor, a galvanic skin response (GSR) sensor, an electromyography (EMG) sensor, an electrodermal activity sensor, and an electrodermal response sensor.

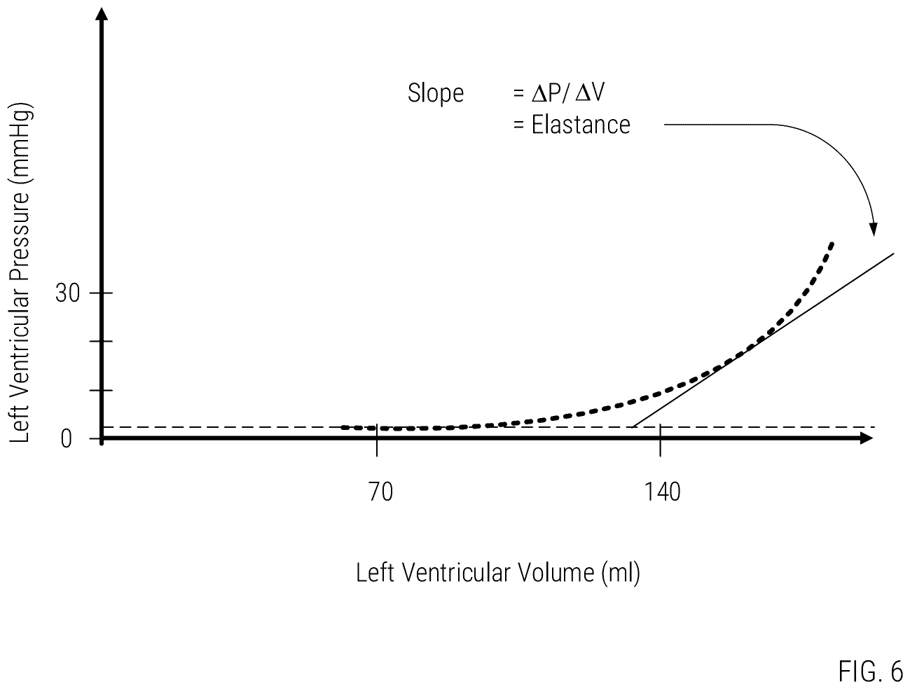

Other efforts have suggested using the size and shape of the pulse as a metric for hydration. Weak pulses are associated with severe dehydration. However, for the proposed purpose of maintaining or optimized physiological performance, pulse size is an inadequate approach. Pulse size, which is often parameterized as height, width, or area under the curve (AUC), is influenced by vasodilation of the peripheral vasculature as well as hydrostatic pressure. Variation in body temperature, or even temperature at the local site of the sensor, will strongly affect pulse size due to changes in arterial tone. Additionally, a simple arm raise will dramatically alter both the size and shape of the pulse. Hickey et al has quantified the type and magnitude of change as illustrated in FIG. 6 of their publication examining the impact of arm raise on PPG signals, Hickey, M., J. P. Phillips, and P. A. Kyriacou. “The effect of vascular changes on the photoplethysmographic signal at different hand elevations.” Physiological measurement 36.3 (2015): 425. A second paper by Hickey explicitly explored the changes of pulse shape in response to arm raises and found pronounced morphological changes. Hickey, M., Phillips, J. P., & Kyriacou, P. A. (2016). Investigation of peripheral photoplethysmographic morphology changes induced during a hand-elevation study. Journal of clinical monitoring and computing, 30(5), 727-736. Thus, pulse size measurements will be limited by vasodilation and arm position, and pulse shape measurements will be strongly affected by the relation of the measurement site relative to the heart.

Perfusion methods have also been used to assess hydration. The most common method used clinically is the capillary refill test. The capillary refill test is initiated by applying pressure to a fingernail for 5 seconds. Following pressure release, the observer examines the time needed for the color of the nail to return to normal. If it takes longer than 1 to 3 seconds, dehydration may be present. Methods based on a similar principle use frequency-or amplitude-based analysis of the PPG signal to determine a so-called “perfusion index”, which assesses the strength of the arterial pulse relative to other signals (often the non-pulsatile mean or “DC” signal). Such a method is disclosed in US Patent Application 2013/0261468 A1, by Semler and Scott, entitled “Non-invasive portable dehydration diagnostic system, device and method.” Similar to approaches based solely on pulse size or shape, perfusion-based methods are limited by sensitivity to the position of the sampling site relative to the heart, the local temperature of the sampling site (which alters tone) and the perfusion of the sampling site, which can be uncorrelated with overall hydration status.

Sweat-based assessments have focused on several measurements including the amount of sweat lost as well as concentration measurements in the sweat. Proposed measurements include determination of cortisol, while other use measurement methods developed for cystic fibrosis test to measure sodium levels. The use of SW[Na+] (sweat sodium concentration, mmol/l) has been studied and the review article by Villiger, et al. describes a number of limitations including the need for a baseline measurement, influenced due to aldosterone, and sympathetic nervous system, Villiger, M., et al. “Evaluation and review of body fluids saliva, sweat and tear compared to biochemical hydration assessment markers within blood and urine.” European journal of clinical nutrition 72.1 (2018): 69.

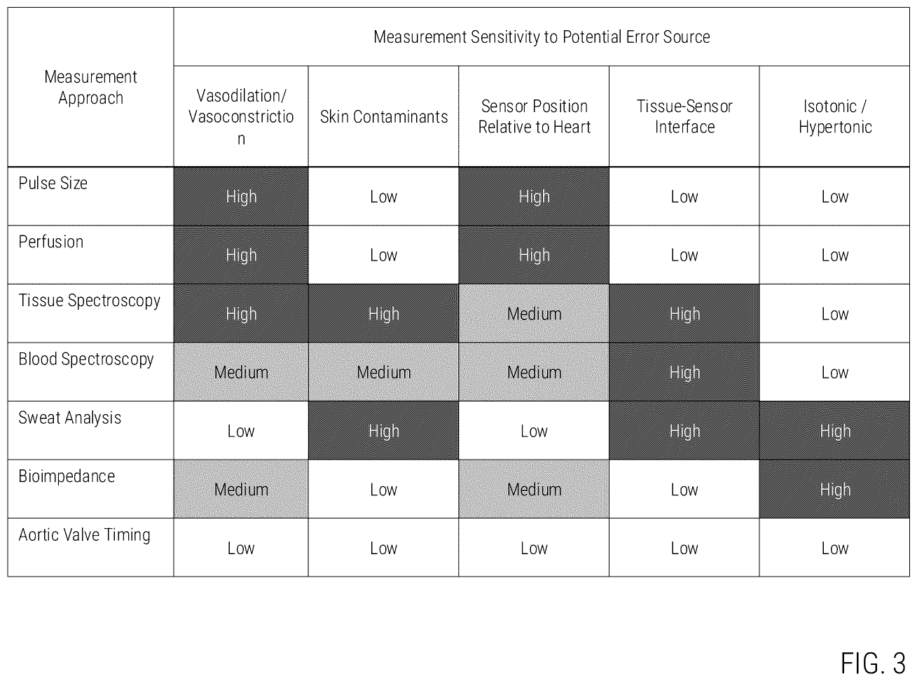

Novel Approach. The current approach is a significant departure from prior efforts largely focused on determining water concentration. Instead, the invention is based on the time course of aortic valve opening and closure. Embodiments of the current invention can be used during activities of daily living. Thus, the approach can be relatively insensitive to changes in vasodilation, sampling site location relative to the heart, skin contaminants (such as sweat), and subtle changes in the tissue-sensor interface. Because the current invention is based on the detection of aortic valve opening and closing, events which are generated centrally by the heart, the conditions at the peripheral sampling site (e.g., arterial tone, precise interface with the sensor, and position relative to the heart) have relatively little or no influence. Furthermore, because the measurement approach of the current invention is equally not intrinsically affected by electrolyte concentration, it is capable of detecting both isotonic dehydration (e.g., caused by water loss in diarrhea) and hypertonic dehydration. (e.g., caused by water loss in sweat). FIG. 3 shows the sensitivity of the previously discussed conventional methods to confounds that are likely be present during typical use. Examination of the table illustrates the value of the invention in the target use environment of everyday living. The ability to determine hydration from the time course of aortic value opening and closing is unique and valuable because it solves many preexisting measurement problems and support embodiments that can be used during activities of daily living.

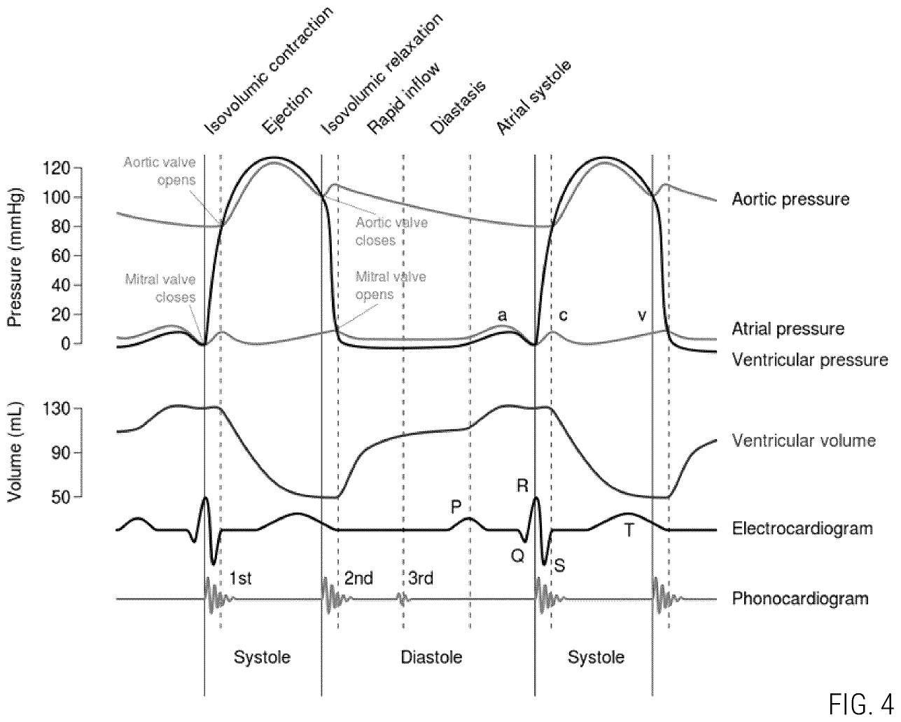

The ability to use the time course of aortic value opening and closing for the determination of hydration requires an understanding of cardiac physiology. The relationship between cardiac function and the time course of aortic valve status is illustrated in FIG. 4. The figure shows a time axis with pressure and volume relationship defined over the cardiac cycle with aortic and mitral valve function illustrated.

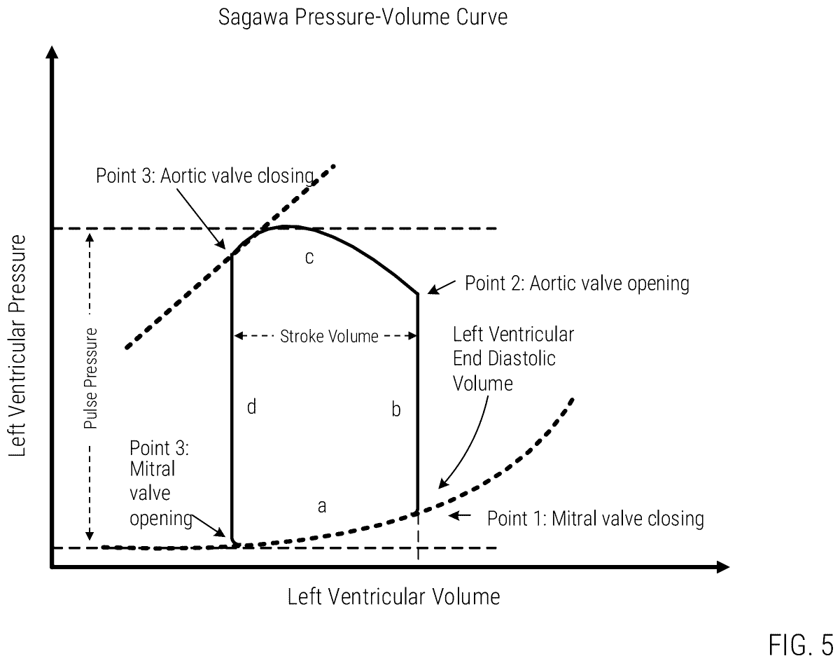

Further axes and relationships are necessary to understand how aortic valve timing relates to hydration status. Sagawa pressure-volume loops (or “PV loops”) create a relationship between pressure and volume with the aortic value status defined as critical transitions in the loop. FIG. 5 is a schematic representation of a PV loop and illustrates the relationship between filling pressure, stroke volume and aortic valve opening and closing. To generate a pressure volume loop for the left ventricle, the left ventricular pressure (LVP) is plotted against left ventricular (LV) volume at successive time points during a complete cardiac cycle. This creates a PV loop as shown in FIG. 5. A single cardiac cycle can be divided into four basic phases: ventricular filling (phase a; diastole with aortic valve closed), isovolumetric contraction (phase b, aortic value closed), ejection (phase c, aortic value open), and isovolumetric relaxation (phase d, aortic value closed). Point 1 on the PV loop is the pressure and volume at the end of ventricular filling (diastole), and therefore represents the end-diastolic pressure and end-diastolic volume (EDV) for the ventricle. As the ventricle begins to contract isovolumetrically (phase b), the LVP increases but the LV volume remains the same, resulting in a vertical line (all valves are closed). Once LVP exceeds aortic diastolic pressure, the aortic valve opens (point 2) and ejection (phase c) begins. During this phase the LV volume decreases as LVP increases to a peak value (peak systolic pressure) and then decreases as the ventricle begins to relax. When the aortic valve closes (point 3), ejection ceases and the ventricle relaxes isovolumetrically. The LV volume at this time is the end-systolic (i.e., residual) volume (ESV). When the LVP falls below left atrial pressure, the mitral valve opens (point 4) and the ventricle begins to fill. Initially, the LVP continues to fall as the ventricle fills because the ventricle is still relaxing. However, once the ventricle is fully relaxed, the LVP gradually increases as the LV volume increases. The width of the loop represents the difference between EDV and ESV, which is by definition the stroke volume (SV).

The opening of the aortic valve defines the end of diastole and the closure of the aortic valve defines the end of systole, thus the time separation of these two events is directly proportional to stroke volume.

For the purpose of quantifying hydration, the relationship between changes in hydration status and changes in the aortic valve timing must be quantifiable. The ability to quantify hydration is based on mechanical properties of the left ventricle and the resulting pressure volume relationships. At start of diastole, the blood entering the ventricle is filling the ventricle and the degree of pressure change is minimal. This period of filling can be referred to as the unstressed filling phase. The situation is like filling an empty balloon. However, as the ventricle fills further the heart begins to stretch and the pressure increases dramatically. This phase of filling can be referred to as stressed filling as the heart wall is becoming stressed. The mechanical properties of the heart are designed to prevent a burst or failure situation. The resulting pressure-volume curve has highly nonlinear relationship as shown in FIG. 6. During a condition of dehydration, the filling pressure into the heart is lower and the heart operates in the more linear region of the pressure-volume curve. The ratio of unstressed filling to stressed filling will be higher than a condition of euhydration or over-hydration.

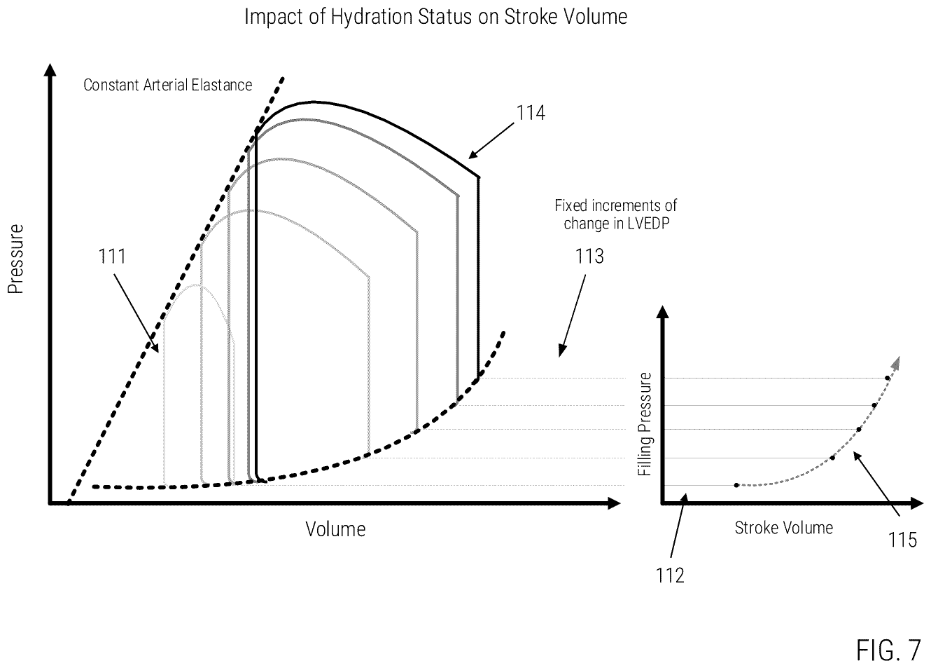

FIG. 7 illustrates the impact of the unstressed to stressed filling ratio and the resulting impact on stroke volume. Pressure volume curve 111 illustrates a lower left ventricular end diastolic pressure curve with most of the filling occurring under unstressed condition as evidenced by the small change in left ventricular pressure. This curve is representative of a dehydrated state, where circulating volume has been reduced. The stroke volume associated with pressure-volume loop 111 is shown by the segment labeled 112. For illustration purposes, the left ventricular end diastolic pressure was increased in equal increments, as shown by the horizontal lines, 113. The resulting pressure volume curves of each increment in left ventricular end diastolic pressure are illustrated and the resulting stroke volume illustrated in the graph to the right of the pressure volume curves. Pressure volume curve 114 is at the highest left ventricular end diastolic pressure and corresponds to a condition of over hydration. Curve 115, shown with the dashed line is a curve connecting the end points of the various stroke volumes as defined by the above pressure-volume curves and their associated stroke volumes. The illustration clearly shows a highly nonlinear response of stroke volume with filling pressure, which can be used to determine hydration.

Thus, the ejection time, defined as the duration between the opening and the closing of the aortic valve, directly corresponds to the stroke volume defined by the separation of vertical lines b and d in FIG. 5, which can be used to assess hydration status as shown in FIG. 7.

The current invention effectively transforms changes in hydration into an observable time-based measurement that support embodiments that can be used during activities of daily living.

Determination of Aortic Valve Closure. There are several sensor technologies capable of sensing aortic valve opening and closing. However, the ability to reliably detect aortic closure in a noninvasive and wearable device presents challenges that are specifically addressed by the current invention. A brief overview of sensing technologies is provided, followed by an in-depth discussion of some innovative elements of embodiments of the present invention that facilitate reliable aortic closure determination.

In medical settings, aortic valve closure is frequently determined from a central artery pressure waveform, as measured by Doppler ultrasound or invasive catherization. The closure of the valve produces a downward notch in the aortic blood pressure, known as the incisura, due to a brief backflow of blood. The incisura is readily detected with ultrasound and catheterization, however such measurement systems are inconvenient and inconsistent with in the activities of daily living.

Optical sensors measuring changes in blood volume, commonly referred as photoplethysmography (PPG) sensors, have the potential to measure aortic valve closure and are significantly more amenable to use in wearable devices. PPG sensors can be used on various locations on the body including one or more fingers, one or more ears, one or more wrists, chest, or forehead. PPG devices can also include image-based systems with spatial resolution over one or more dimensions.

Methods such as laser Doppler flowmetry, tonometry, pulse transduction, and impedance cardiography (the measurement of electrical conductivity of the thorax), that are sensitive to changes in volume, flow, or pressure related to the cardiac cycle, can also be used to acquire signals indicative of aortic valve closure.

An alternative group of methods, sensitive to the vibrations associated with the movement of the aortic valve includes, phonocardiography, ballistocardiography, seismocardiography. Phonocardiography (PCG) is a method of detecting the sounds produced by the heart and blood flow. Similar to auscultation, PCG is most commonly measured noninvasively from the chest with a microphone. Ballistocardiography (BCG) and seismocardiography (SCG) are both methods for studying the mechanical vibrations that are produced by the cardiac cycle. BCG is a method where the cardiac reaction forces acting on the body are measured. SCG, on the other hand, is a method where the local vibrations of the precordium (the region of the thorax immediately in front of the heart) are measured.

The preceding examples do not comprise an exhaustive list of technologies that can sense physiological changes associated with opening and closing of the aortic valve, but illustrate the variety of methods that have the potential to be used in the current invention.

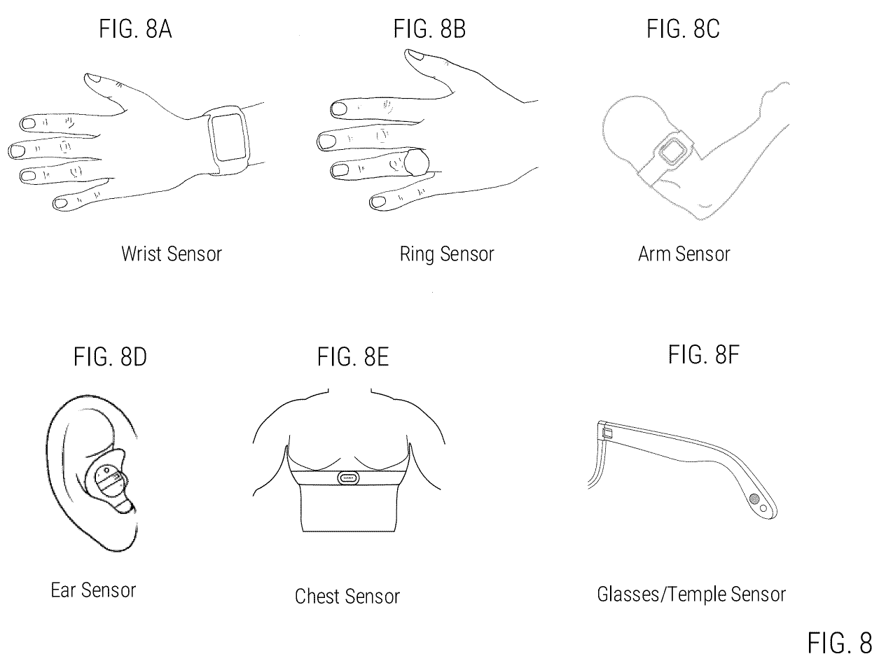

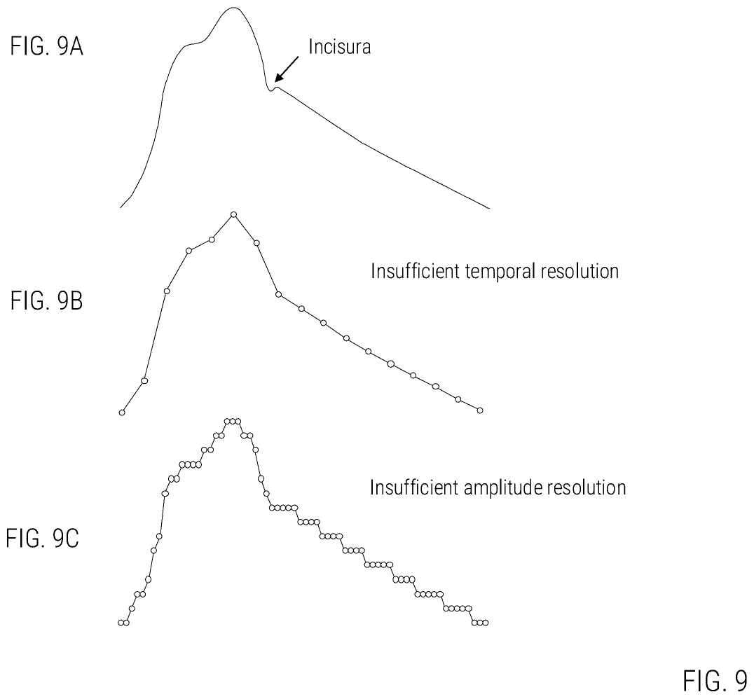

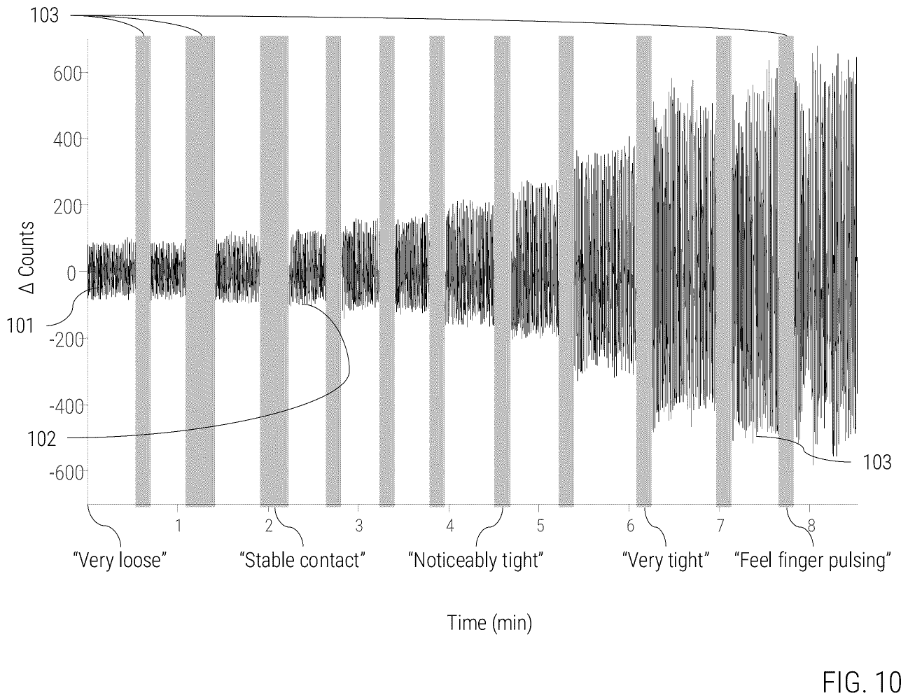

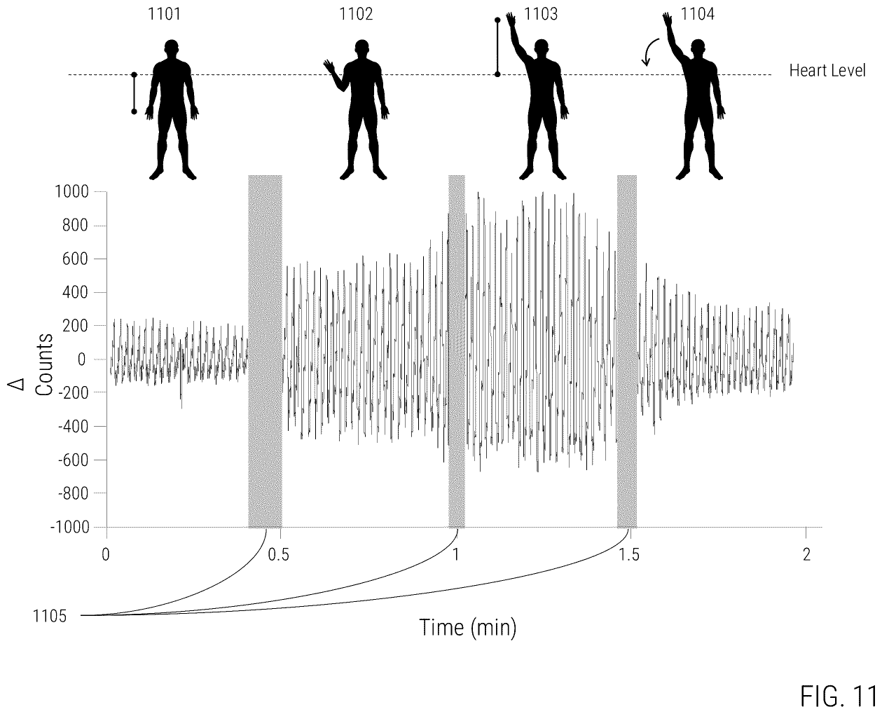

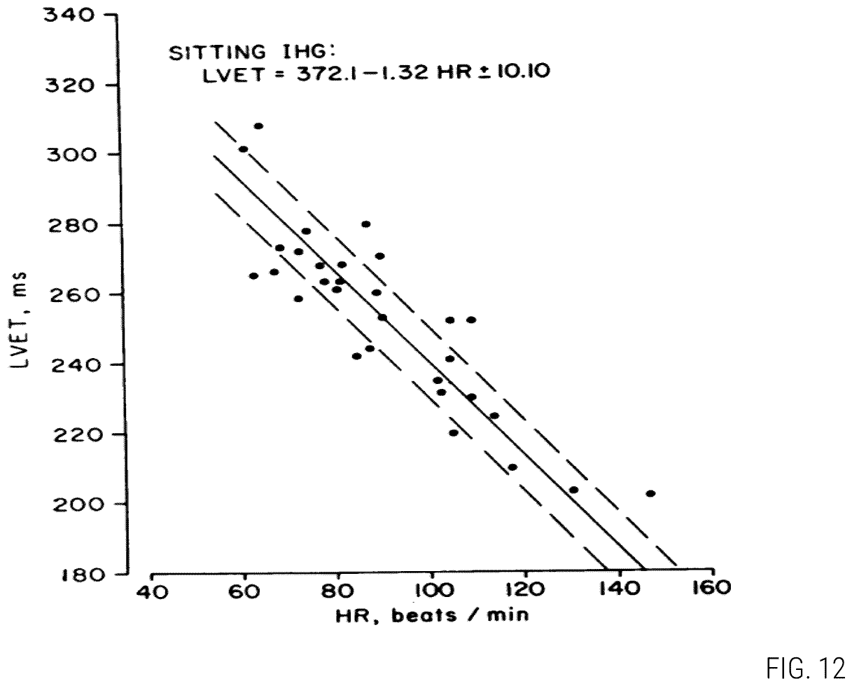

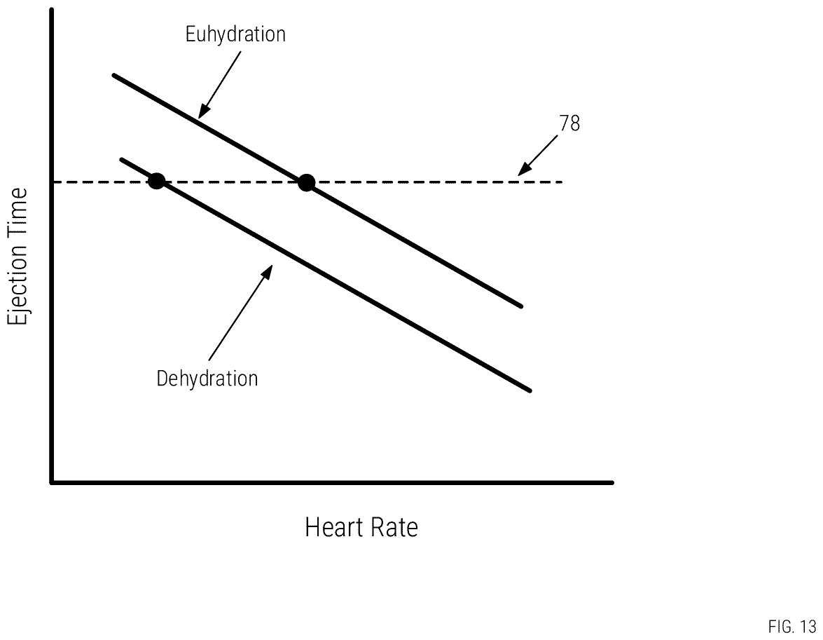

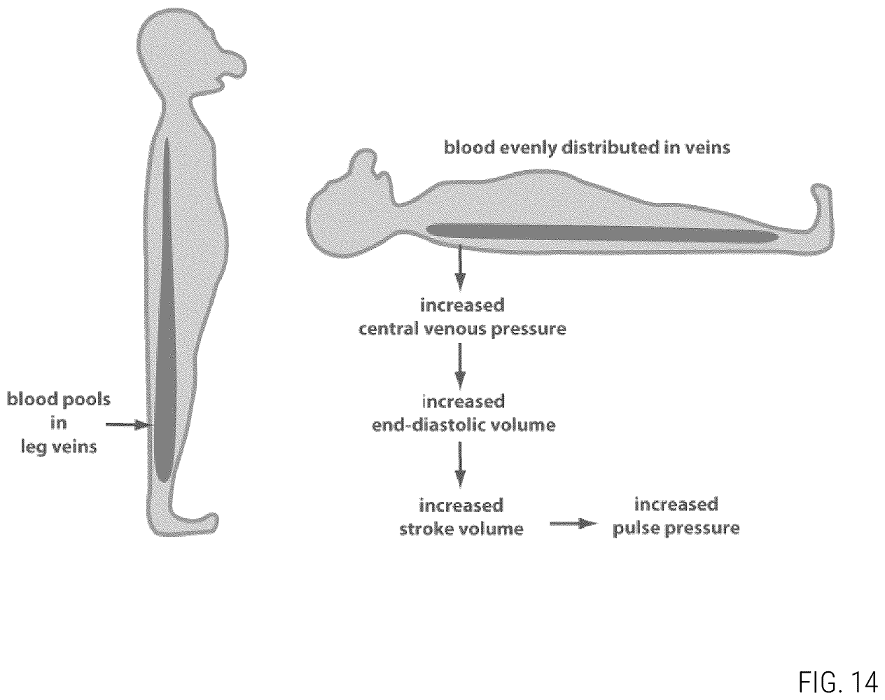

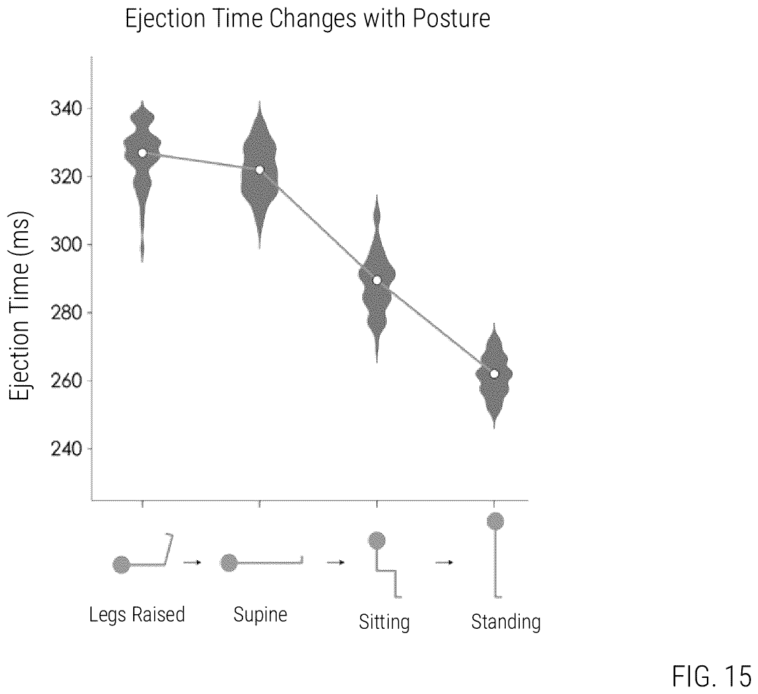

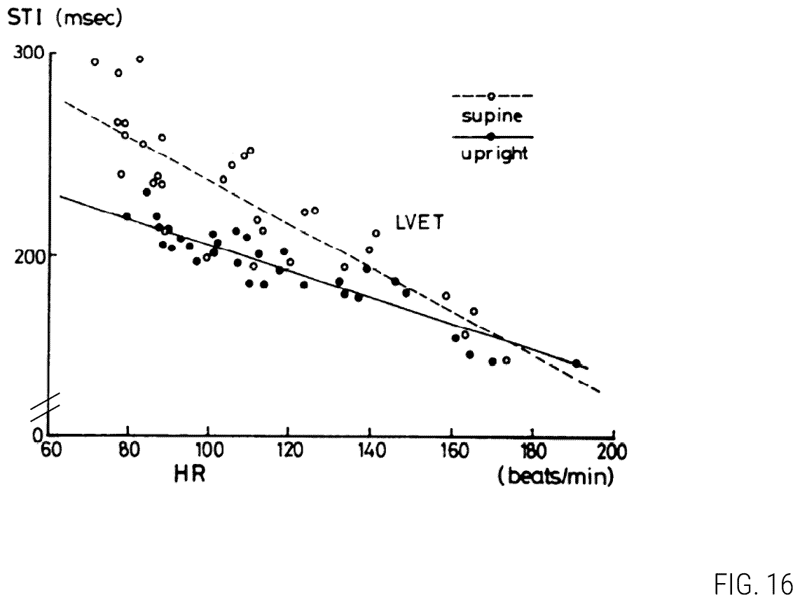

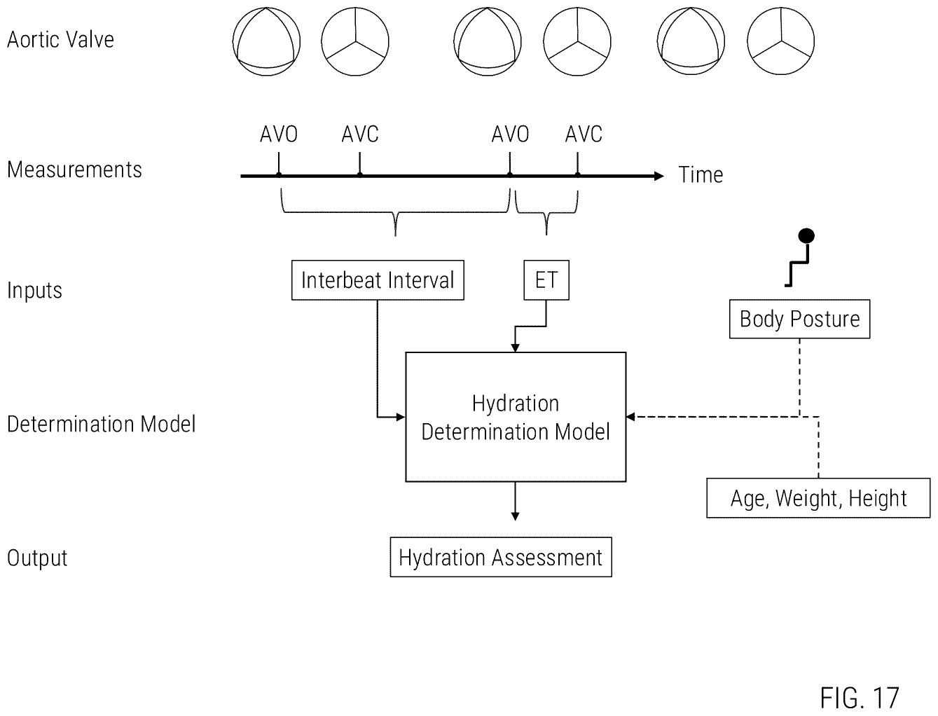

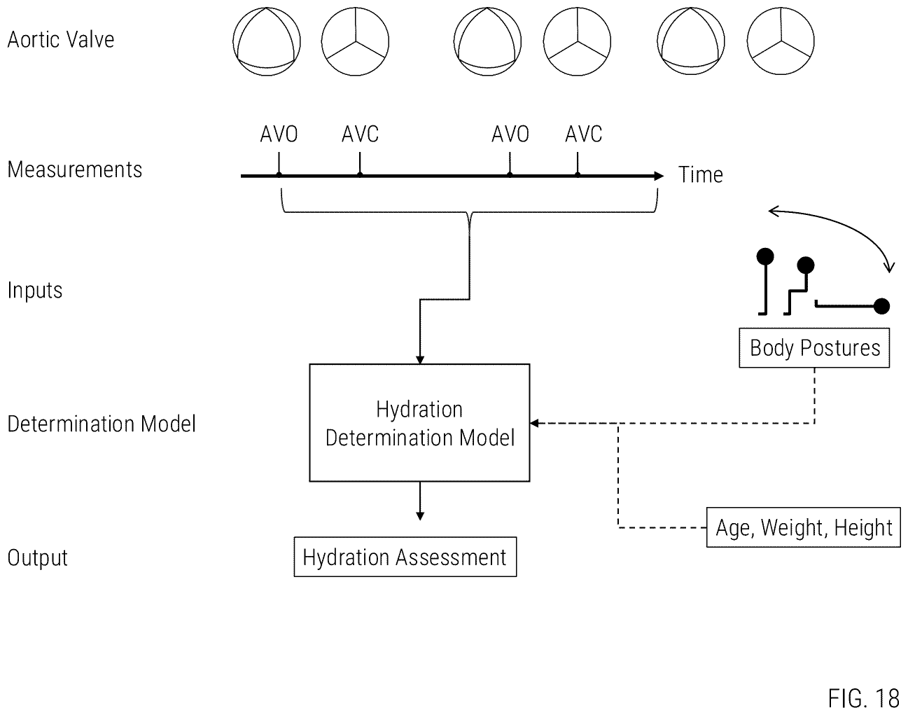

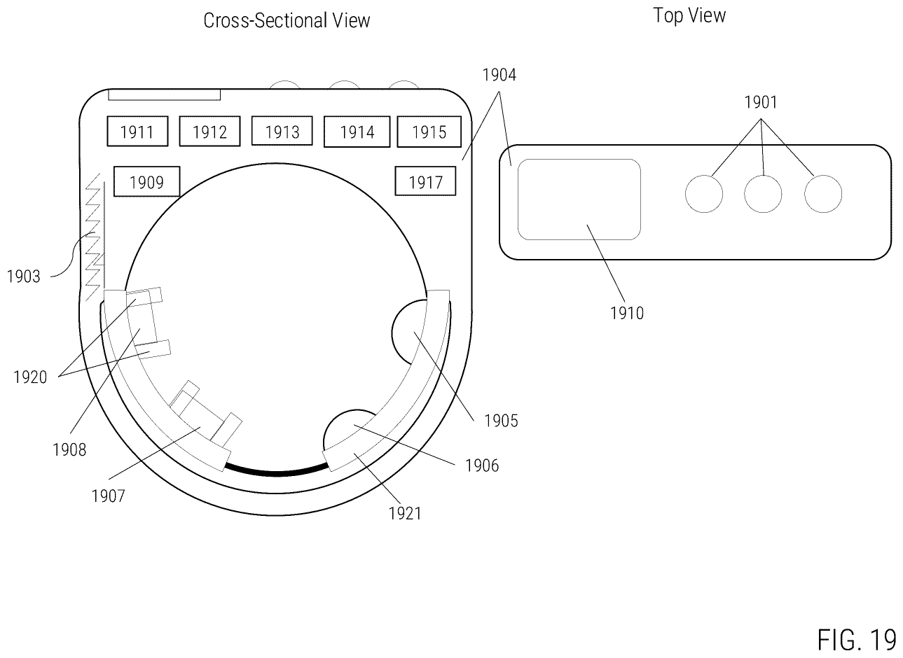

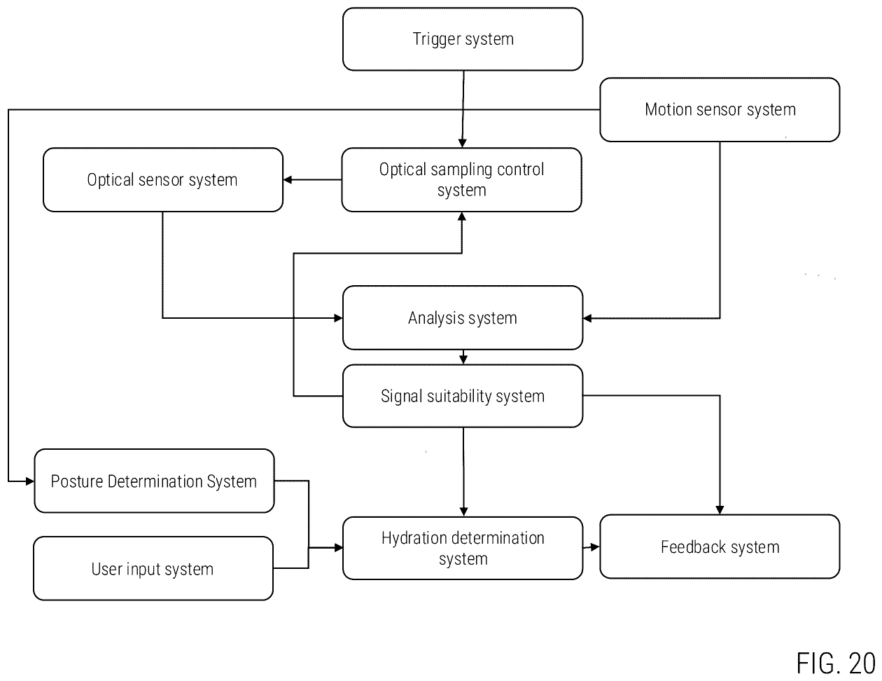

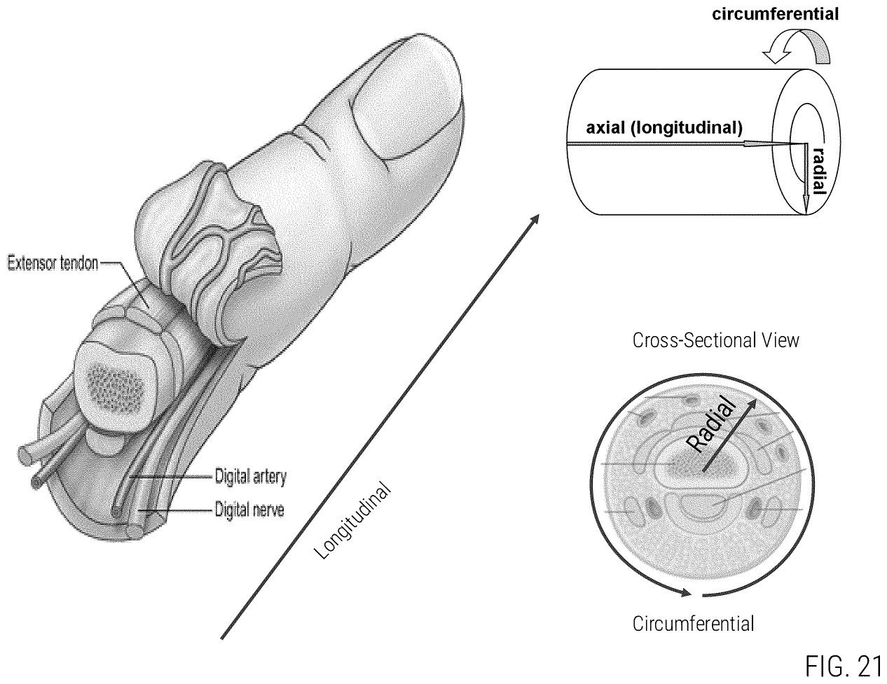

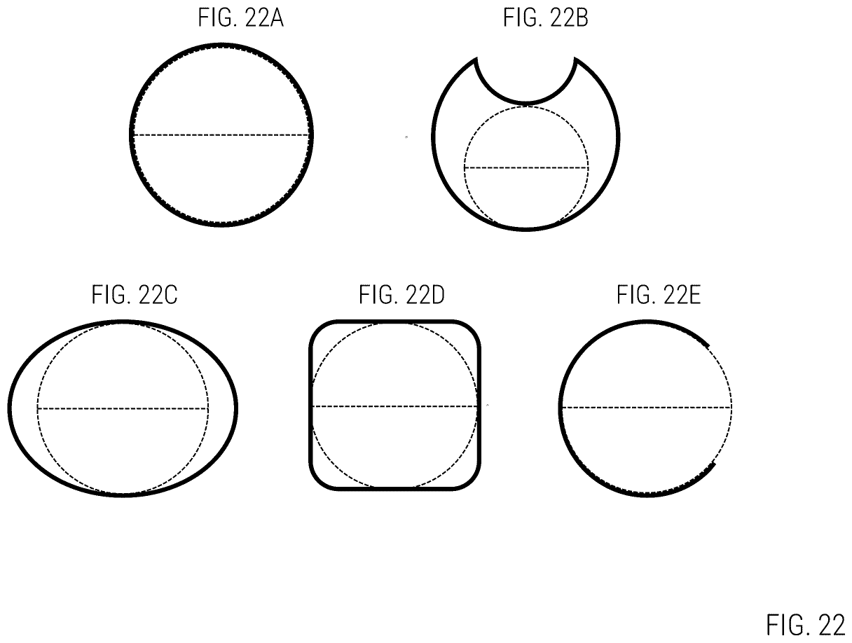

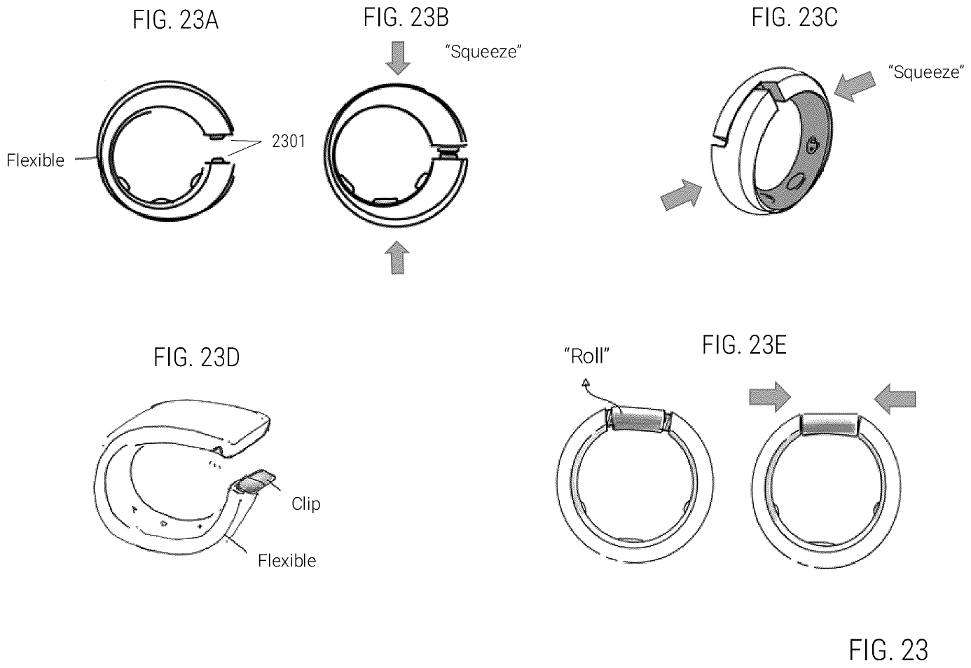

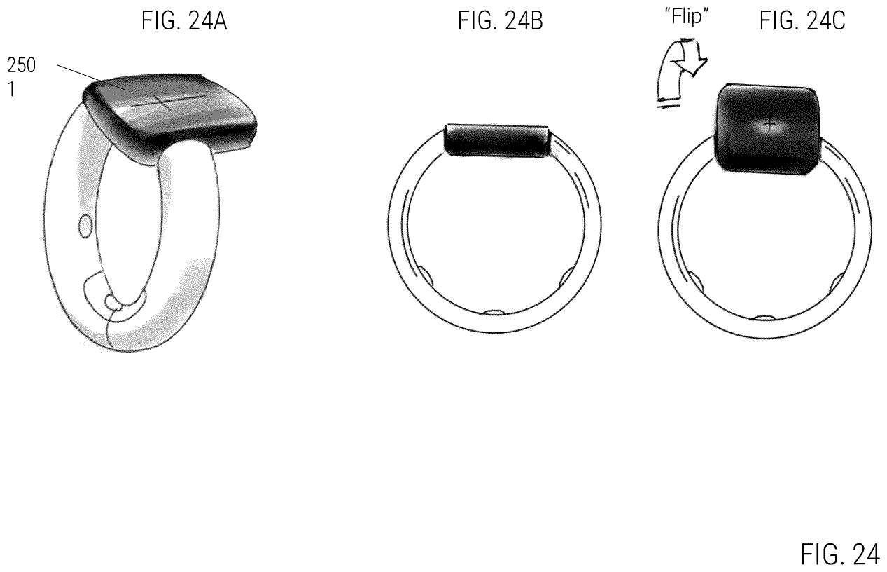

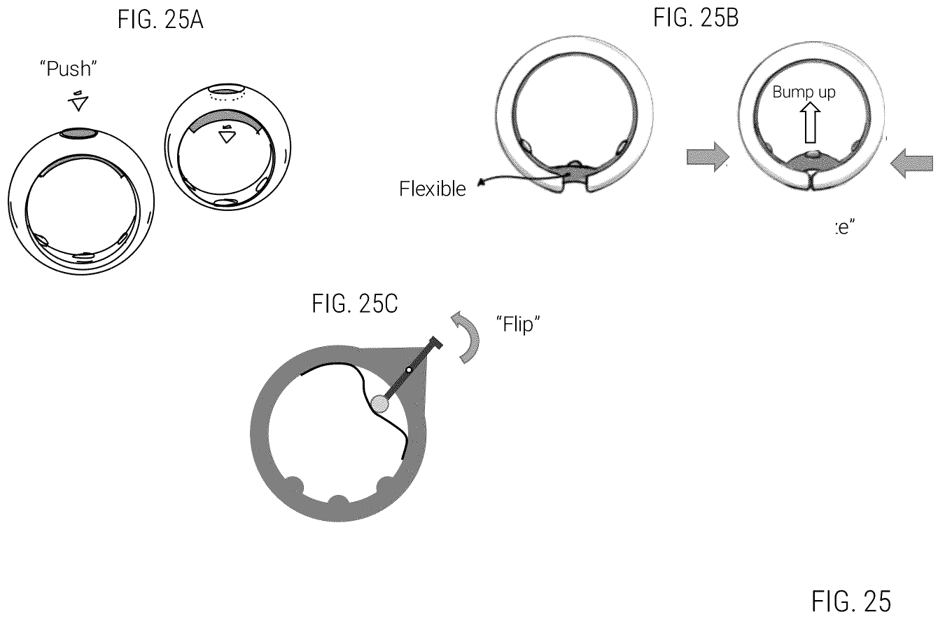

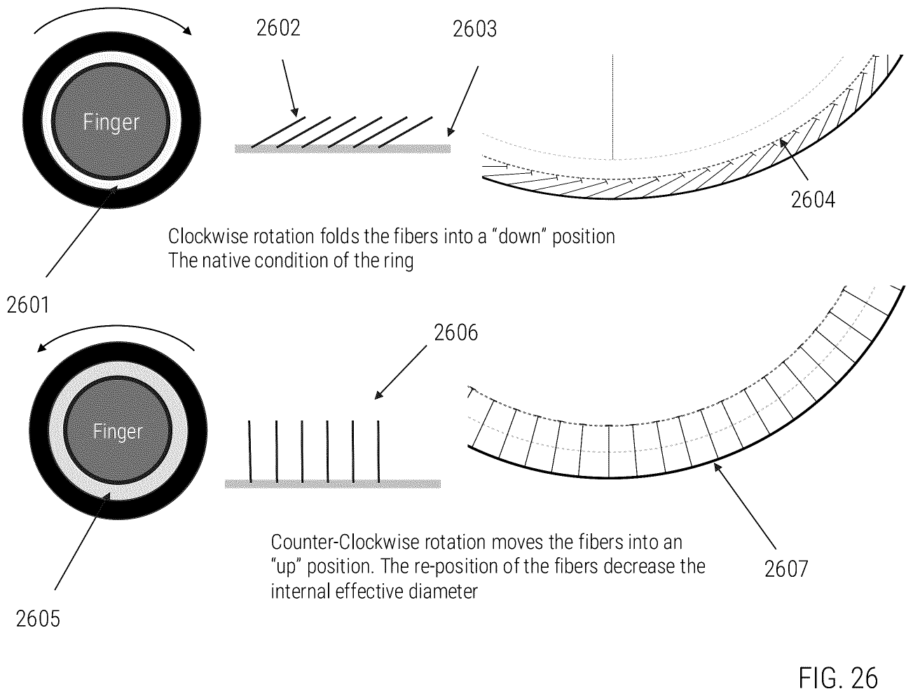

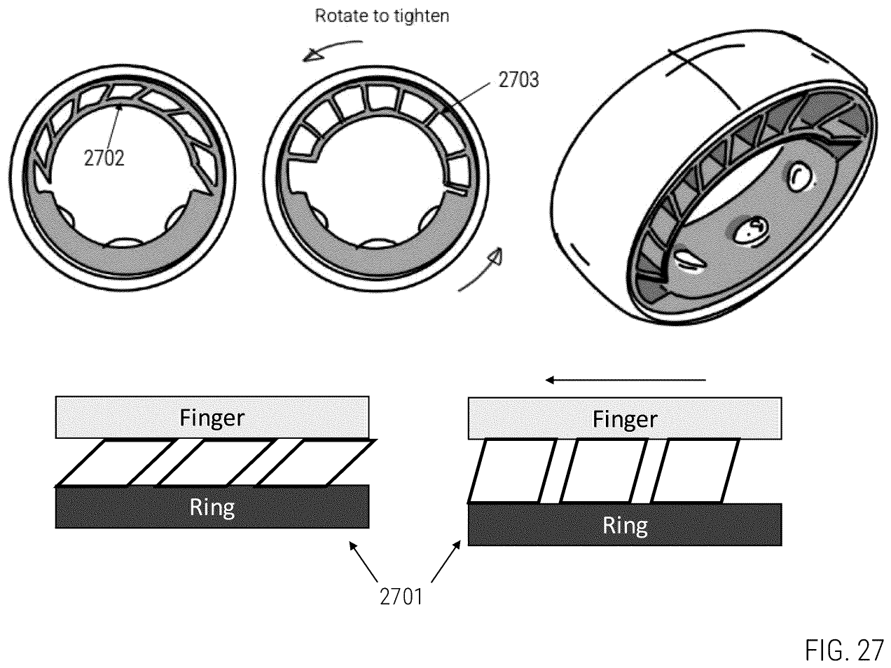

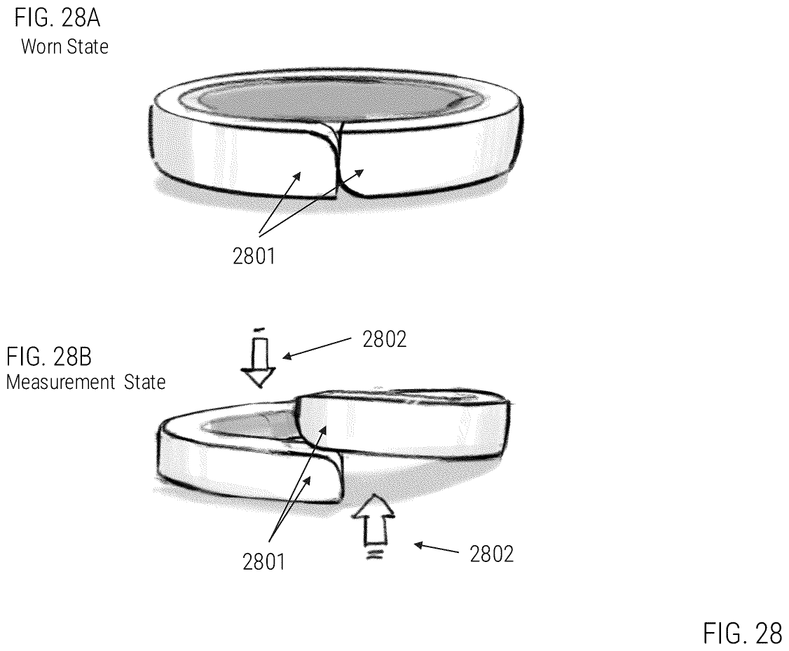

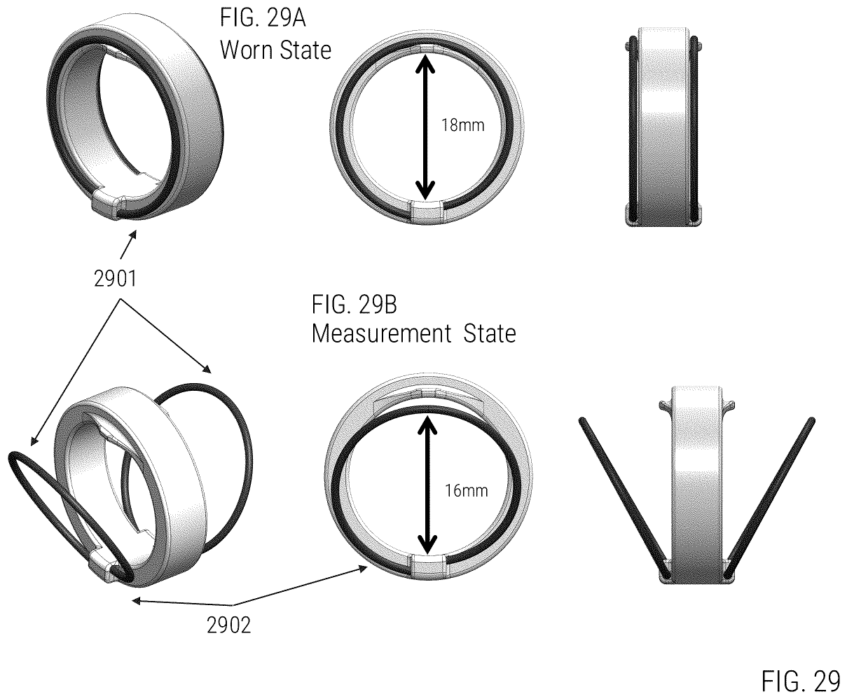

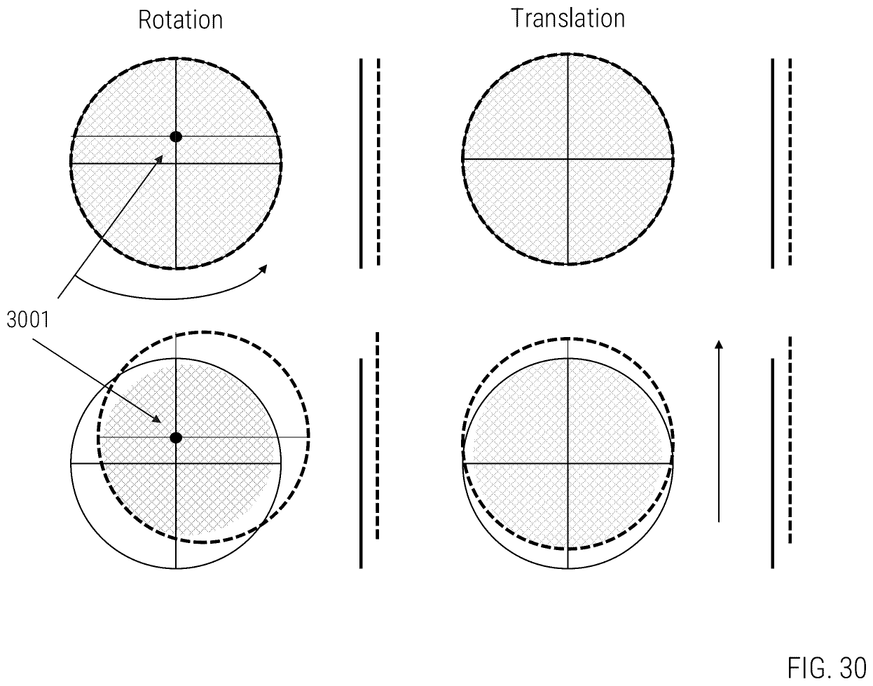

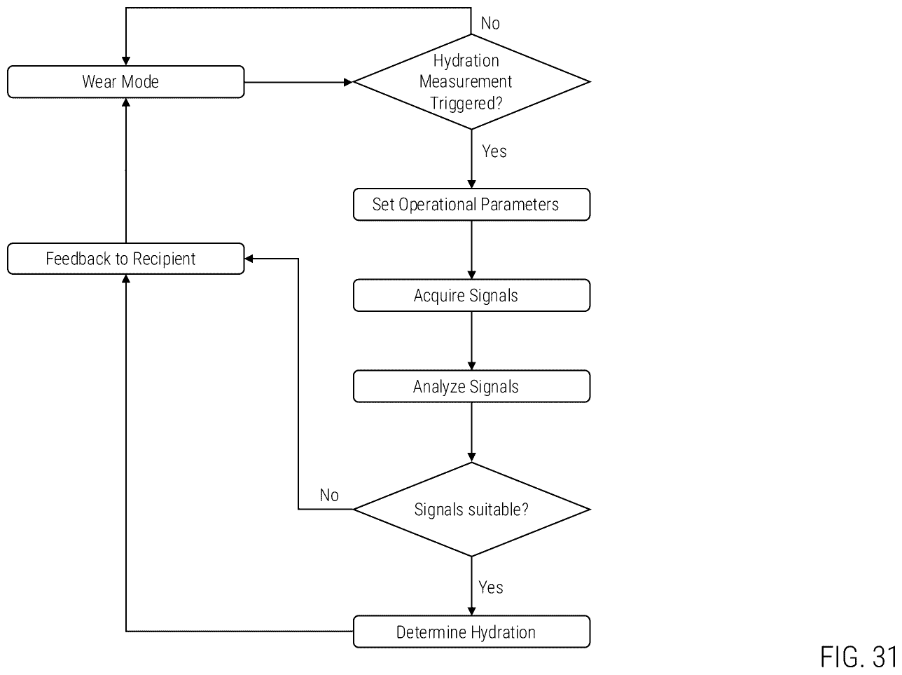

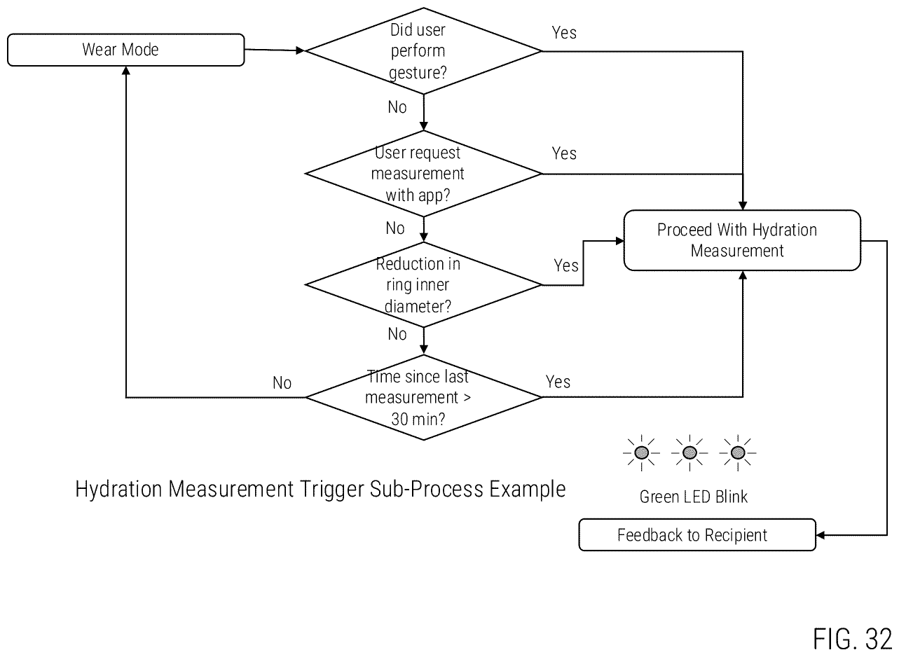

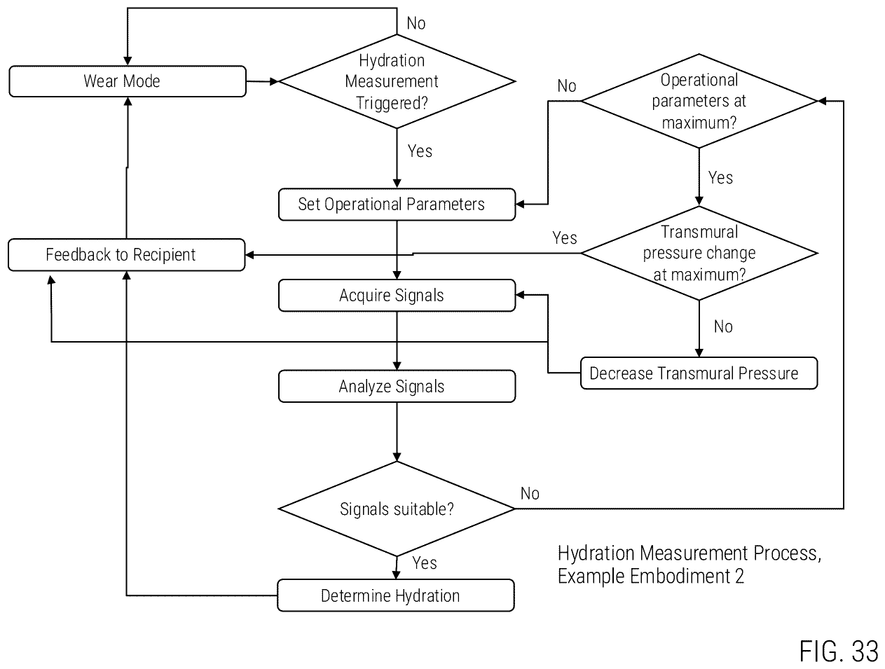

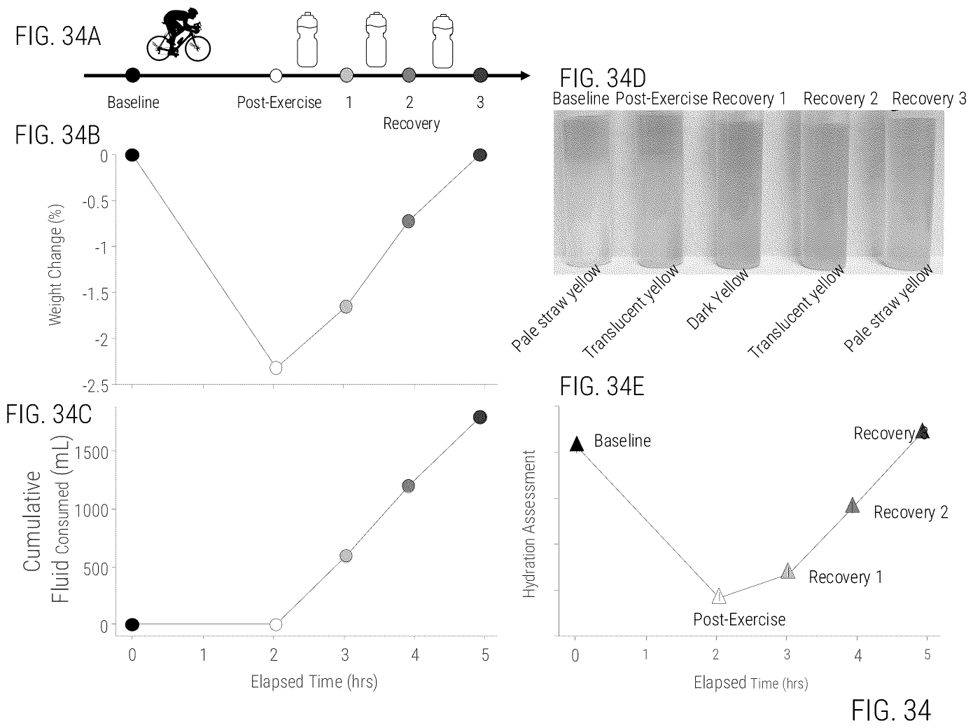

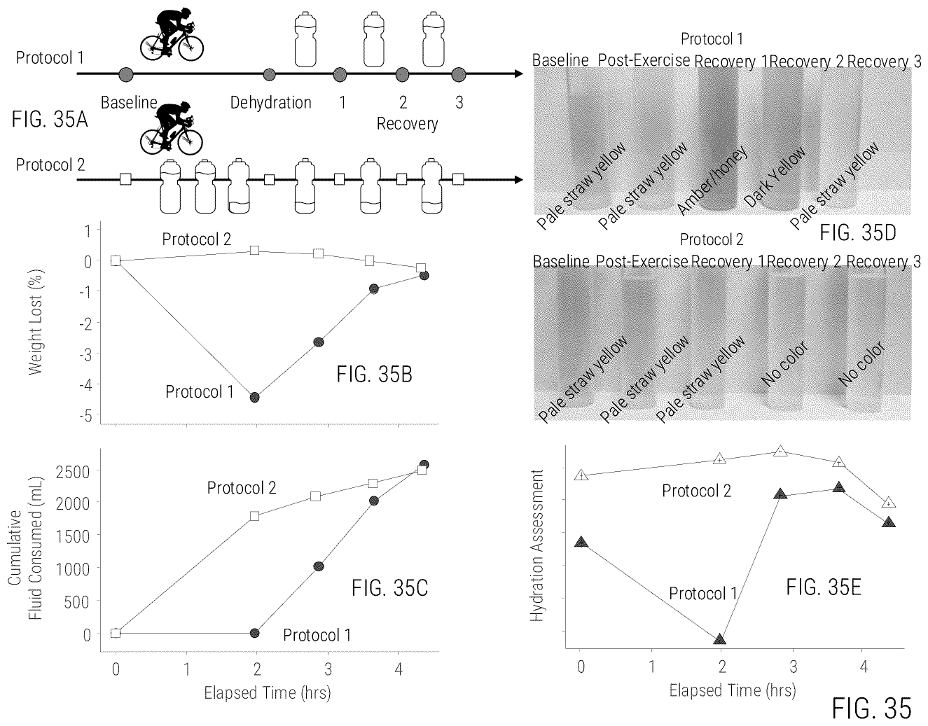

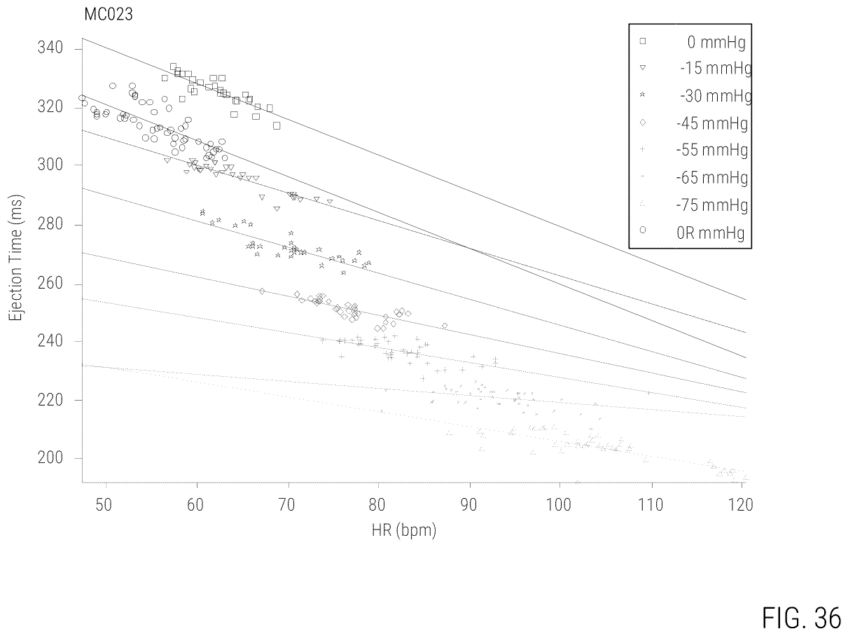

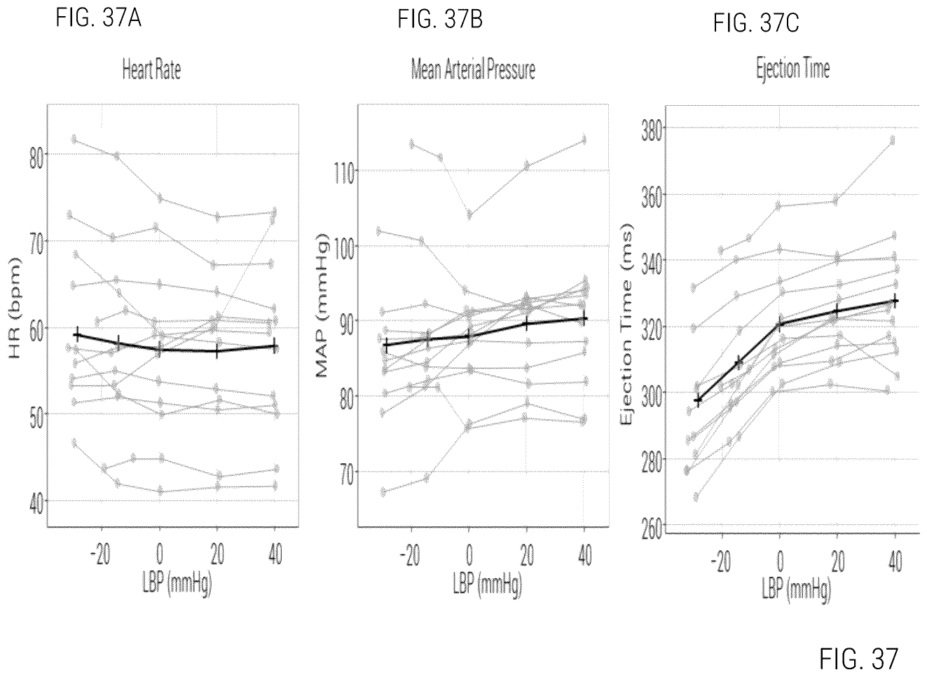

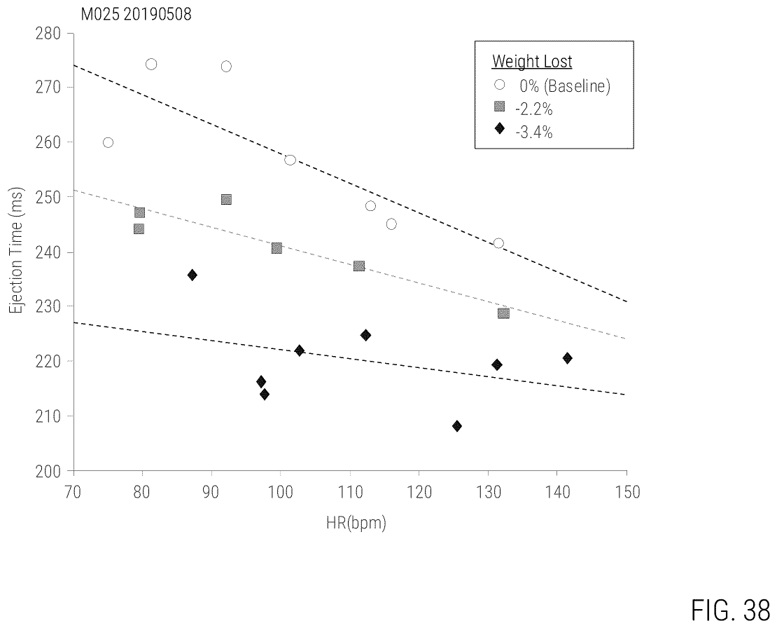

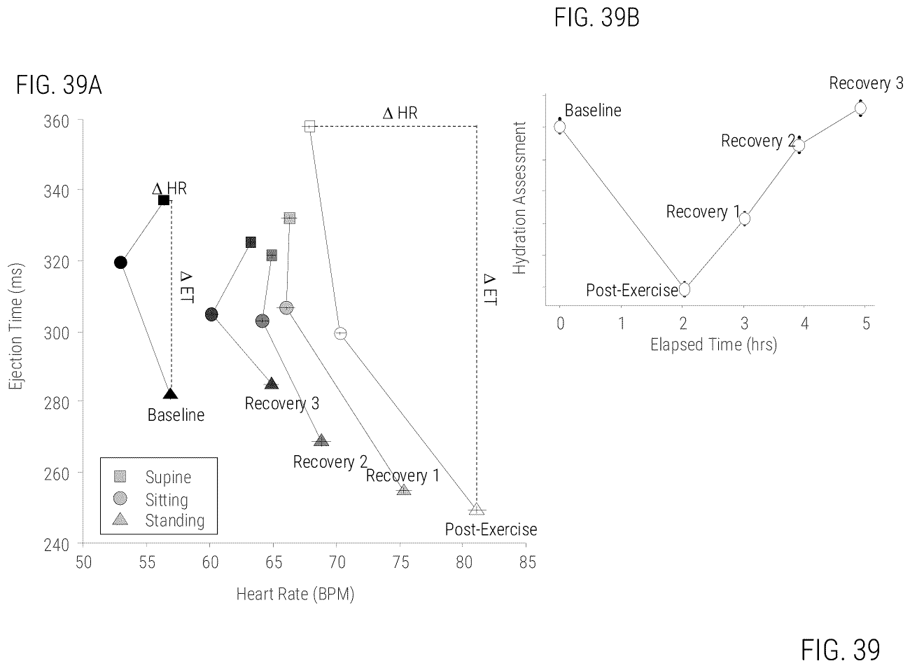

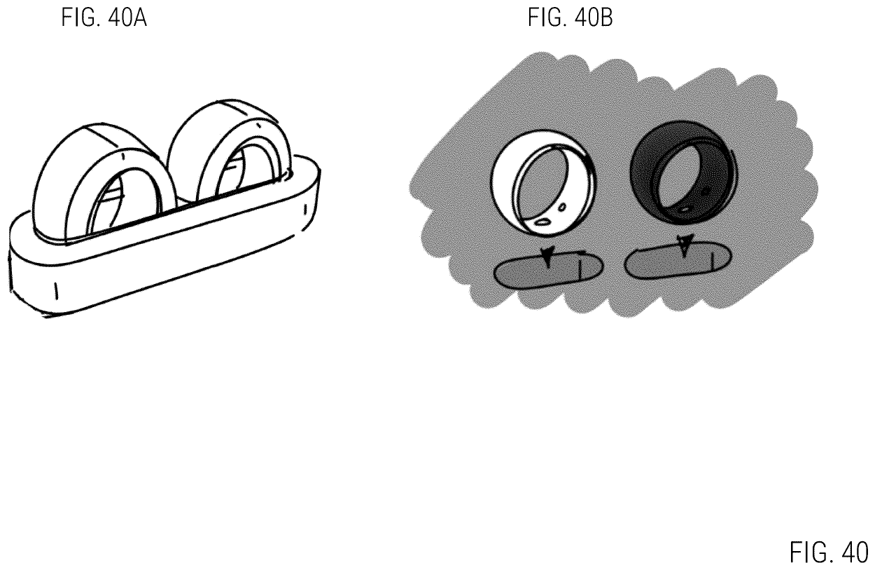

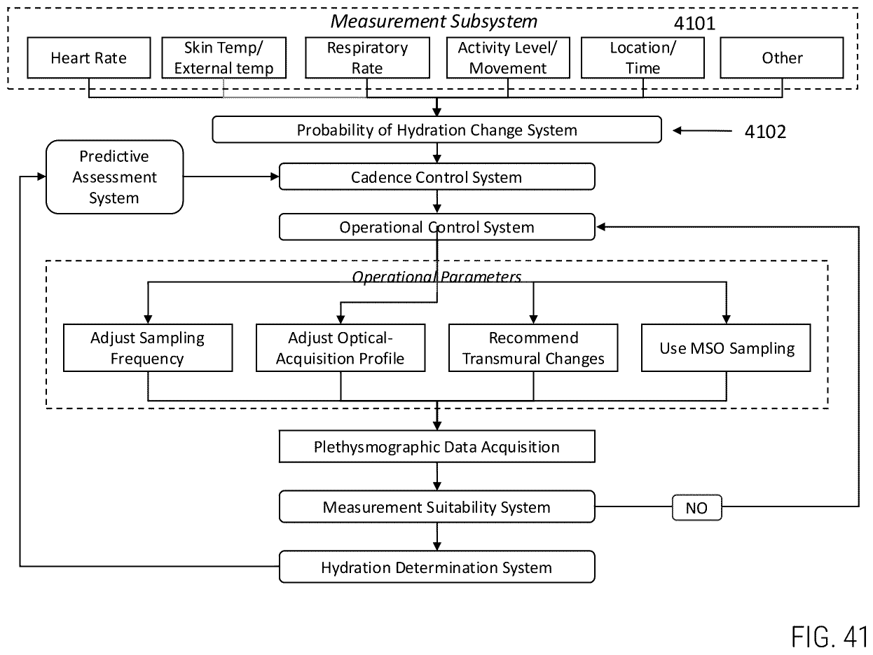

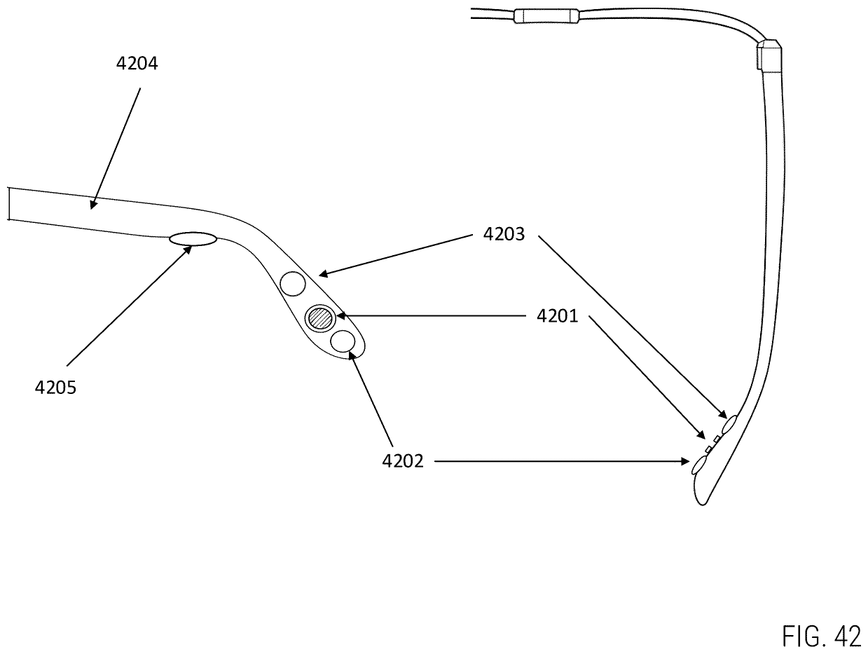

FIG. 8 illustrates several measurement locations and wearable housing where such sensors can be used to create data streams containing information of aortic valve opening and closing without interfering with the activities of daily living. FIG. 8A is a wrist-based reflectance sampling system. FIG. 8B is a ring-based sampling system that can have transmission sampling, reflectance or both. FIG. 8C is an arm-based sampling system. FIG. 8D is an ear-based sampling system. The system can have sensors that sample the external ear, such as the tragus or the inter ear canal. FIG. 8E is a chest-based reflectance sampling system.

FIG. 8F is a temple tip-based reflectance sampling system. Temple tips, also known as earpieces, are the parts of eyeglass frames located at the end of the temples (arms) that rest behind the ears in contact with the skin. A hydration-monitoring system can be realized by sampling optical signals at the eyeglass temple tip—the portion of the frame that rests behind and slightly above the auricle. This location offers several intrinsic advantages for physiologic sensing. The skin in the post-auricular and parietal region is thin, richly vascularized, and situated above the level of the right atrium, which minimizes hydrostatic and venous pooling effects that can confound peripheral measurements at the wrist or ankle. Properly fitted eyeglasses maintain a nearly fixed spatial relationship to this skin surface during routine activities and most athletic movements, so motion artifacts are markedly reduced compared with other wearable sites. Because eyeglasses are already worn for vision correction, fashion, or eye protection, integrating a sensor in the temple imposes no additional adherence burden on the user and does not alter established wearing habits. The relatively flat geometry of the temple tip also simplifies optical coupling and packaging of solid-state components. The illustration of FIG. 8F shows a single source detector pair. The detector is the circle region that is crosshatched, and the emitter is the circle without crosshatching.

Beyond the sampling locations illustrated in FIG. 8, any skin surface that permits acquisition of plethysmographic signals, whether by reflection, transmission, trans-illumination, speckle, radar, or related modalities, constitutes a viable sampling site for the hydration-monitoring system. Suitable locations include, without limitation, (i) the forehead, brow, nose bridge, cheeks, and periorbital rims (integrated into caps, visors, headbands, goggles, face shields, respirators, masks, helmets, or smart-glasses frames); (ii) the external ear, ear canal, tragus, concha, and mastoid areas (earbuds, hearing aids, behind-the-ear receivers, temple arms, or adhesive patches); (iii) the scalp, vertex, temporal, or occipital, with sensors embedded in hats, helmets, hairbands, sweatbands, or scalp patches; (iv) the neck, lateral carotid triangle, suprasternal notch, or nuchal surface, via collars, neckbands, lanyards, or cervical kinesiology tape; (v) the upper limb, including finger tips, finger bases, inter-phalangeal creases, dorsal and palmar wrist, volar and dorsal forearm, antecubital fossa, triceps region, and deltoid insertion, supported by rings, thimbles, gloves, watch cases, wristbands, armbands, compression sleeves, adhesive strips, or clothing panels; (vi) the thorax, sternum, clavicular saddle, axilla, precordium, and inframammary fold, using chest straps, compression vests, sports bras, or ECG patch hybrids; (vii) the abdomen and flanks, with integration into belts, abdominal binders, smart waistbands, or ostomy-style adhesive wafers; (viii) the lower limb, spanning groin crease, quadriceps, hamstrings, popliteal fossa, medial and lateral malleoli, Achilles insertion, instep, plantar arch, toe webs, and toe tips, facilitated by shorts, tights, knee sleeves, socks, insoles, anklets, or toe-ring sensors; and (ix) sites on the back, scapular plane, lumbar ridge, or sacrum, embedded within posture braces, adhesive patches, smart textiles, hydration packs, or backpack straps. Each anatomic zone may exploit native vasculature (e.g., superficial temporal, facial, carotid, radial, posterior tibial, or plantar arteries) and can accommodate single-point, multi-point, or imaging-array configurations, thereby ensuring that the invention is not limited to any specific wearable form factor or body region but extends to essentially any locus where optical, mechanical, electrical, or radio-frequency plethysmography can be reliably captured with the sensor in contact, or in proximate near-field coupling, with human tissue.

The previous sensing technologies and the sensor locations in FIG. 8 may resemble wearable devices currently available and designed for other purposes, but such “off the shelf” sensors cannot be used to reliably determine aortic valve closure. As an example, numerous currently available wearable PPG systems are designed to determine heart rate or heart rate variability. This determination requires only the measurement of signals or events associated with aortic valve opening. At the PPG measurement site, aortic valve opening manifests as a rapid increase in blood volume corresponding to the arrival of the pulse. Conventional wearable PPG heart rate monitors often use frequency or spectral analysis of the PPG signal to identify periodic changes in the PPG signal consistent. An example of this approach is disclosed in U.S. Pat. No. 10,178,973 B2 entitled “Wearable Heart Rate Monitor.” Venkatraman discloses that a user's heart rate can be determined from an optical PPG signal using a process that outputs the “periodic component” of the PPG signal. Venkatraman does not teach to determine the events of aortic valve closing nor aortic valve opening, per se.

Thus, devices designed to measure other physiological parameters are not suitable for the reliable determination aortic valve closure. In peripheral pulse waveforms, the signal associated with aortic valve closure is 50 to 100 times smaller than the signal associated with aortic valve opening. Accurate detection of aortic closure with a wearable device requires a carefully considered measurement system that incorporates physical and operational features distinct from those conventionally used to detect other physiological parameters. The following sections detail these physical and operational features, with some details and examples specific to optical sensing technologies. One of skill in the art will recognize that many of the same principles can be used with alternative measurement technologies.

Sampling Resolution. The ability to assess hydration at a level useful to the user requires high resolution of the change in blood volume, flow, or pressure in both the temporal domain and the signal amplitude domain. In the temporal domain, a sampling rate near or above 100 Hz facilitates determination of the events of aortic valve opening and closing to within 10 ms. Lower sampling rates can increase the error in ejection time calculation and hence subsequent hydration assessment. In the signal amplitude domain, amplitude resolution should be sufficient to resolve the changes associated with aortic closing, which are on the order of 1% of the magnitude of changes related to aortic valve opening. In embodiments where acquired signals are digitized through an analog-to-digital converter, the bit-depth of the system should be sufficiently high such that signals related to the aortic valve closure are not lost with discretization.

In optical systems, the amplitude of signals associated with aortic valve closure can be enhanced by increasing the intensity or brightness of light used, provided that detectors and other aspects of the data acquisition system are not saturated. Light intensity can be increased with increased LED drive current or by increasing the number of LEDs in use, or both. Signal amplitude can also be increased by configuring additional operational parameters of the optical system, such as the integration time (length of time that photons are acquired at the detector). In wearable devices that are intended to be worn for prolonged periods battery life is always a concern. Because LED activation can produce a significant drain on batteries, overall LED intensity and duration of use can be considered prudently and used only as needed.

FIG. 9 demonstrates the effects of insufficient resolution on determination of aortic valve closure. FIG. 9A shows the pressure trace of a cardiac pulse sampled with high resolution in both domains. Aortic valve closure is determined from the incisura in the pressure wave. When the pulse is sampled with low temporal resolution of 16 Hz in FIG. 9B, the ability to determine the timing of the incisura is significantly degraded. A sampling rate of 16 Hz is common for heart rate determination in wearable devices but is insufficient for aortic valve closure determination. In FIG. 9C, the temporal resolution is improved but the resolution of the amplitude has been strongly degraded due to discretization. Here again, the precise timing of the aortic valve closure is difficult to discern. Thus, embodiments of the invention comprise a measurement system with the resolution in both the time and signal amplitude dimensions to enable detection of the aortic valve closure.

Sampled Vessels. For measurements of pressure, volume, or flow, the incisura signal associated with aortic valve closure will be largest at more proximal arterial segments and will dissipate along the vasculature tree. The signal will be more apparent in larger tri-layered vessels such as arteries and arterioles than in the largely inelastic capillaries.

As it relates to optical systems, near-infrared light, which is absorbed weakly by blood and tissue, can penetrate deeply (>1 mm) into the tissue and interact with larger vasculature segments. This contrasts with shorter wavelengths in the visible range, in particular green light, which is strongly absorbed by pigments in skin, blood, and tissue. For green light, the capillary bed effectively serves as a screen to prevent direct interaction with larger vessels. Thus optical sensors employing shorter wavelengths (green or blue light) with short optical paths that interact with capillaries have less sensitivity to the signal associated with the aortic valve closure than sensors employing longer wavelengths (red and infrared) with longer optical paths that interact with more proximal arterial segments.

The physical configuration of light emitters and detectors in an optical system also plays an important role in determining the optical path length and the type of vessels that are sampled. When the emitters and detectors are placed in close proximity (e.g., separated by <5 mm) the detected photons are more likely to have interacted primarily with superficial vessels in the capillary bed. When the detector is at greater separation from the emitters, the photons that reach the detector are more likely to have interacted with deeper tissue containing more proximal arterial segments. Because shorter wavelengths of light in the visible range are so strongly absorbed by tissue, emitters and detectors must be in relatively close proximity to enable sufficient photon detection. However, longer wavelengths in the red and near-infrared range can be used when emitters and detectors are physically separated by more than 10 mm, supporting optical paths where the majority of photos interact with artery and arteriole segments. To further encourage interaction with such vascular segments, emitters and detectors can be arranged such that the optical path traverses known anatomical locations of arteries. For example, in the fingers, the prominent palmar digital arteries run longitudinally along the sides of fingers, close to the volar surface of the hand. Therefore, more volar (ventral) placement of emitters and detectors can be advantageous to sample the arteries.

Notably, maximization of SNR related to aortic valve closure might not be equivalent to maximizing SNR for aortic valve opening. Because green light is so strongly absorbed by blood, the magnitude of the pulsatile signal associated with aortic valve opening can be significantly larger than the signal obtained with longer wavelengths. In addition, green light sensors are less influenced by venous compartments due to their shallow penetration depths, reducing sensitivity to some motion-related artifacts. The result is that for conventional wearable systems measuring heart rate and heart rate variability, green light can be optimal. This is taught, for example, by Maeda et al (Maeda, Y., Sekine, M., & Tamura, T. (2011). The advantages of wearable green reflected photoplethysmography. Journal of Medical Systems, 35(5), 829-834).

For the purpose of a hydration measurement, the system seeks to maximize the SNR related to aortic valve closure by deeper sampling of larger vessels such as arteries and arterioles that maintain a stronger signal of aortic valve closure.

### Tissue-Sensor Interface

A prominent noise source for all sensing technologies is movement of the measurement device relative to the tissue. Device design can mitigate this issue, by protruding sensing components relative to the surface of the device such that they maintain consistent contact with the tissue.

For optical systems, device design can also reduce noise caused by ambient or stray light. Preferably, only light rays that have interacted with the tissue will be captured by the detector. However, light rays that have merely bounced off the skin or other surfaces, or that originate from environmental sources might also be detected and constitute a source of noise. Embodiments of the invention can include light-management components that control or restrict detected light. These components include but are not limited to physical blockers placed around the detector to limit the angles of light rays that can reach the photosensitive surface, optical elements (such as optical fibers or lenses) placed in front of the photodetector that similarly restrict the numerical aperture of the detector, and polarizers placed between the light source and detector at orthogonal orientations to limit detection of light rays that have only undergone surface reflections.

Additionally, ambient light cancellation (ALC) can be incorporated to remove interference from ambient light. ALC approaches detect light both when LEDs are active and inactive, allowing for compensation of signals in LED active periods by LED inactive periods. An example of ALC circuitry is disclosed by Kim et al (Kim, Jongpal, et al. “Ambient light cancellation in photoplethysmogram application using alternating sampling and charge redistribution technique.” 2015 37th Annual International Conference of the IEEE Engineering in Medicine and Biology Society (EMBC). IEEE, 2015).

### Size of Physiological Signal

Beyond changes to the operational parameters and configuration of the optical sensor system, the SNR can be increased by changing the size of the pulsatile signal.

The size of arterial pulsations can be increased by decreasing the vascular transmural pressure (TMP), that is, the pressure gradient across artery walls. At least three mechanisms are responsible for this enhancement in pulse size with TMP decrease: (1) decreases in TMP trigger arterial dilations through the local venoarterial reflex (VAR), (2) decreases in TMP trigger the myogenic response, i.e., the relaxation of the smooth muscles in artery walls, and (3) because vessel compliance is a function of TMP, decreases in TMP increase arterial compliance such that a given change in arterial pressure results in a large change in arterial volume. TMP can be reduced by applying external pressure at the measurement site or raising the elevation of the measurement site relative to the heart to decrease hydrostatic pressure.

Optimal external pressure is typically greater than the venous pressure but less than the arterial diastolic pressure; pressures beyond this point will begin to occlude flow and distort the pulse waveform. Based on the work of Balijepalli et al (2014), 95% of individuals aged 18-99 years have a diastolic pressure above 60 mmHg. If the sampling site is near or below the level of the heart, external pressures in the range of 50 mmHg can be appropriate to increase the magnitude of arterial pulsation; Error! Reference source not found. and Error! Reference source not found. (discussed below), the effect of TMP on pulse size is graded, thus any appreciable external pressure (e.g., greater than 5 mmHg) will produce some increase in the pulse. (Balijepalli, C., Lösch, C., Bramlage, P., Erbel, R., Humphries, K. H., Jöckel, K.-H., & Moebus, S. (2014). Percentile distribution of blood pressure readings in 35683 men and women aged 18 to 99 years. Journal of Human Hypertension, 28(3), 193-200).

By way of comparison, Brophy-Williams et al (2014) report that “sports compression” tights exert an interface pressure in the range of 5 to 30 mmHg, depending on the region of the lower limb and body posture. Coltman et al (2015) find that standard bra straps can exert ˜40 mmHg of pressure in static positions, and as much as 75 mmHg during high intensity activities. Thus, an external pressure of ˜50 mmHg is not outside the range of pressures exerted by standard garments, though is likely beyond the range of pressures produced by garments or devices intended to be intrinsically comfortable. (Brophy-Williams, N., Driller, M. W., Shing, C. M., Fell, J. W., & Halson, S. L. (2015). Confounding compression: The effects of posture, sizing and garment type on measured interface pressure in sports compression clothing. Journal of Sports Sciences, 33(13), 1403-1410). (Coltman, C. E., McGhee, D. E., & Steele, J. R. (2015). Bra strap orientations and designs to minimise bra strap discomfort and pressure during sport and exercise in women with large breasts. Sports Medicine—Open, 1(1), 21).

Examples of the effect of TMP on pulse size are shown in Error! Reference source not found. and Error! Reference source not found. Error! Reference source not found. shows the detector signal from an adjustable PPG ring worn at the base of the finger. The signal has been band-pass filtered to focus on the pulsatile component. Roughly every 45 s, the ring is tightened incrementally on the wearer's finger via a ratcheting mechanism on the ring band. These tightening events are denoted by gray rectangles 103. The wearer's reported subjective experience associated with the different levels of tightness are indicated below the graph. Initially in period 101, the ring is reported by the user to be “very loose” and the magnitude of the pulse is ˜100 detector counts. After several tightening events the user reports that the ring makes “stable contact” with the finger. The pulse size at this period (102) is ˜150 counts. After this point, each tightening event increasingly changes the TMP through applied external pressure, as evidenced by the increase in pulsatile signal size. When the ring is reported by the user to be “very tight”, the pulse size increases to ˜1000 counts (period 103). After further tightening, the user reports feeling pulsations in the finger, an indication that the external pressure is approaching arterial diastolic pressure. Cumulatively, the tightening events produced a 10% reduction in the circumference of the ring and created a 10-fold increase in signal size is due to the decrease in arterial TMP caused by the increased external pressure at the sampling site.

Error! Reference source not found. shows a second example of the effect of TMP on pulse size, in this case using manipulations in hydrostatic pressure to alter the TMP. Error! Reference source not found. shows a band-pass filtered detector signal from a PPG ring worn at the base of the finger. The ring size is constant throughout the experiment, but the subject undergoes changes in arm positions, indicated by gray rectangles 1105. In period 1101, the arm hangs in a relaxed position at the subject's side. The sampling site is estimated to be 50 cm below the right atrium of heart, resulting in ˜37 mmHg of additive pressure distending the walls of the veins and arteries, due to the hydrostatic pressure exerted by the vertical columns of blood in these vessels. The pulse size in this period is just under 400 counts. In period 1102, the subject raises their hand such that the sampling site is roughly level with the shoulder. The change in vertical displacement with respect to the heart decreases the hydrostatic pressure, decreasing the TMP accordingly. The pulse size therefore increases by more than a factor of 2 to nearly 1000 counts. In period 1103 the subject extends their arm to a comfortable position above their head. The sampling site is now an estimated 67 cm above the right atrium, resulting in a hydrostatic pressure of roughly −50 mmHg. This reduces the TMP, which causes a further increase in the pulse size to roughly 1500 counts. In period 1104, the subject slowly lowers their arm down. As would be expected, the pulse size gradually decreases.

Decreasing TMP at the sampling site provides the additional benefit of reducing physiological signals that are unrelated to aortic valve closure. A large source of plethysmographic noise is venous blood. Since the venous system operates at relatively low pressures, it is quite susceptible to the local effects of volume perturbation during motion. The venous blood in the vascular bed will be easily deformed during subtle motion, changing light absorption and producing a significant source of in-band noise. This noise source can be managed by reducing the venous TMP to below zero, effectively collapsing the veins such that their volume is minimized. This not only stabilizes the venous contribution to vascular volume, but also reduces the overall absorbance of light by non-pulsatile sources.

The magnitude of the pulse signal can also be enhanced by increasing the cross-sectional area of the arteries and arterioles at the sampling site via vasodilation. This can be achieved by warming the tissue at the sampling site, for example, with a heating element embedded in the apparatus.

### Management of Differences in User State

An effective hydration assessment system can manage differences in user states, or differences across individuals, that are unrelated to changes in hydration status. The following paragraphs describe differences in physiological states that can represent potential confounds for hydration assessment, as well as approaches to effectively manage such differences.

### Heart Rate

Some embodiments of the invention provide the ability to determine hydration in the presence of heart rate changes. Heart rate is influenced by many variables, including altitude, age, physical activity, temperature, stress, alcohol, and stimulants such as coffee. Thus, a useful hydration assessment system can provide accurate assessments of hydration in the presence of heart rate changes arising from multiple sources.

A change in heart rate can change the ejection time during conditions of constant hydration. The change in ejection time occurs largely because the heart has less time to fill with blood. For the purpose of detecting hydration, in some embodiments these non-hydration related changes can be mitigated by using heart rate as an additional independent parameter to effectively compensate for ejection time alterations such that an accurate determination of hydration is possible.

Error! Reference source not found. shows a typical relationship between heart rate and ejection time at a single hydration level, reproduced from Lance et al. (Lance, V. Q., & Spodick, D. H. (1976). Heart rate—left ventricular ejection time relations. Variations during postural change and cardiovascular challenges. Heart, 38(12), 1332-1338.) Ejection time falls roughly linearly with heart rate, although numerous studies show that the precise relationship varies across individuals and is a function of age, gender, and fitness level.

Error! Reference source not found. illustrates the potential error that can occur if heart rate is not considered in the determination of hydration. The figure shows a relationship between heart rate and ejection time at two different hydration levels. Line 78 shows the potential error of using only ejection time as the metric for hydration. A change in hydration from normal to dehydrated can be missed if the measurements are acquired at a significantly different heart rates. Conversely, an increase in heart rate can be misinterpreted as a decrease in hydration. Some embodiments of the current invention use both heart rate and aortic timing information to effectively determine hydration despite changes in heart rate.

The ability to compensate for heart rate changes can use a simplistic correction for all individuals. However, improved hydration measurements can be possible by using a more refined and user-specific approach. The system can use a “matched cohort” approach based on age, gender, body mass index (BMI) or fitness level. The user can input in such demographic information into the device, or into an application in communication with the device, to support improved heart rate correction.

The hydration assessment system can request that the user undergo a type of heart rate calibration or compensation procedure. Such a procedure can request the user to get a transient elevation in heart rate heart over a measurement period with little or insignificant changes in hydration statues. A more extensive calibration can request the user provide heart rate changes at two different hydration states.

### Body Position

Another potential difference in user state is the level venous return into the heart. When in the supine (lying flat), the large veins in the chest are filled or plump with blood. The additional volume creates an increased pressure as the veins are stretched. The result of a supine position is an increased central venous pressure, increased end-diastolic volume, and increased stroke volume. When standing, the pressure in the large veins in the legs increases. For example, one meter below the heart, the effect of gravity adds about 74 mm Hg of pressure. The change from supine to standing causes venous distension and blood pools in the legs. The resulting translocation of blood to the legs reduces the blood in the central veins, and the cardiac filling pressures drop. Error! Reference source not found. is a pictorial representation of the above physiology.

The impact of body position on ejection time in a constant hydration state is shown in Error! Reference source not found. The figure shows measurement results (ejection time in ms) of moving a subject through 4 positions: legs raised, supine, sitting and standing. The estimated volume changes between legs up and flat is estimated to be 150 ml. As demonstrated, body position changes impact aortic timing and are potential hydration errors. For example, a decrease in venous return due to a position change can mimic a change in hydration.

Thus, the hydration assessment system can be used when individuals are in a fixed body position, e.g., standing. Examination of Error! Reference source not found. illustrates that standing decreases central venous pressure and end-diastolic pressure. Re-examination of Error! Reference source not found. and Error! Reference source not found. shows that the overall sensitivity to hydration changes is greatest at lower end diastolic pressures. Thus, requesting the subject always stand during the hydration measurement (rather than sit or lay down) can improve the accuracy of the hydration measurement.

Alternatively, information about the user's body position at the time of the measurement can be taken into consideration. Body posture can be determined in many ways, including direct measurements, inferred measurements or self-reported measurements.

The effect of heart rate on ejection time interacts with body position. This is shown by Miyamoto et al, reproduced in Error! Reference source not found. (Miyamoto, Y., Higuchi, J., Abe, Y., Hiura, T., Nakazono, Y., & Mikami, T. (1983). Dynamics of cardiac output and systolic time intervals in supine and upright exercise. Journal of Applied Physiology, 55(6), 1674-1681.). The slope relating ejection time to heart in the supine position is steeper than that in the upright (standing) position. Thus, both the body posture and heart rate can be considered in concert to make an effective hydration assessment.

### Changes in Body Position

In addition to controlling for body position, acquiring measurements over different body positions can be used to perform the hydration assessment. Additionally, changes in body position can be used to create a self-referenced measurement where the degree of change between positions is compared or calculated and compared against an existing standard. The comparison standard used can be a general population-based standard that is used for all users. The standard for comparison can be a “matched” standard where the selected standard is based on parameters associated with a cohort of users that match characteristics of the user. For example, potential matching features can include, but are not limited to, gender, age, body mass index, height, physical fitness, use of tobacco, etc. A final standardization can be based on self-determined standards. The user can establish their response when adequately hydrated and a secondary response standard at a defined dehydration level. In use, the self-reference approach can be used to access hydration status in the morning when rising from the bed. The system can use postural transitions from sleeping to sitting to standing as a method for accessing aortic valve timing under three different venous return conditions. The ability to compare day-to-day trends for a single individual enables the system to identify small perturbations in hydration that can influence physiological performance.

### Incorporating Information

Error! Reference source not found. and Error! Reference source not found. illustrate the incorporation of information about user state into the effective determination of hydration status. As shown in Error! Reference source not found. , hydration assessment depends primarily on the time course of aortic valve opening and closing, from which the interbeat interval (IBI; the inverse of heart rate) and ejection time (ET) are determined. Additional inputs can include body posture, which can be assumed based on user compliance, acquired based on input from the user, or determined from sensors, as well as physiological information about the user, such as age, weight and height. These inputs are then used by a hydration determination model to produce the desired output of hydration status.

Error! Reference source not found. represents a variant on the prior example. In this example, the hydration determination model incorporates the time course of aortic valve opening and closing acquired at more than one body posture. As before, the postures can be assumed based on user compliance, acquired based on user input, or determined from sensors. Also as before, demographic information about the user can comprise an additional input to the hydration determination model. Distinct from Error! Reference source not found. , the hydration determination model in this example does not take as input the explicitly calculated ET and IBI; instead, the model utilizes a data representation that contains information associated with aortic valve opening and closing. Deep neural networks, particularly those with convolutional layers or recurrent structures, can be trained to predict hydration status based on a data stream, using information in the data stream that is inherently associated with the opening and closing of the aortic valve. The data stream used by the determination model can be raw (unprocessed), or can be processed, filtered, or transformed, in some manner prior to being entered into the model.

One of skill in the art will recognize that the above approaches to incorporating information for hydration determination comprise limited examples and that many other approaches are possible.

### Example Apparatuses for Hydration Assessment

Error! Reference source not found. shows an illustrative embodiment of an apparatus (1904) capable of making a hydration measurement based on aortic valve opening and closing. In the example embodiment, the apparatus is configured as a ring to be worn on a finger. The apparatus includes one or more of the operational systems described below. Functional element(s) of each system are described, though additional capabilities can also be present.

### Key Systems of Operation

The apparatus includes an optical sensor system comprising one or more emitters (1905 and 1906) and one or more detectors (1907 and 1908). The optical sensor system is used to emit photons into the tissue at a sampling location and detect photons that have interacted with the tissue. In this embodiment, physical blockers 1920 surround the detectors to limit the influence of stray light. The emitters can have the same emitting wavelength or different wavelengths. A given emitter can also represent a package of LEDs, with the capability to emit a plurality of wavelengths. The detectors can be the same or different, with regard to their active area, spectral sensitivity, or other parameters. The optical sensor system can be configured to perform time-division multiplexing and de-multiplexing, such that signals from a plurality of wavelengths can be acquired during the same acquisition period. The optical sensor system can be further configured to perform ambient light cancellation.

A motion sensor system, e.g., accelerometer 1909, is used to obtain motion information at the sampling location. In alternative embodiments, the motion sensor system can comprise sensors that quantify motion in at least one dimension, such as accelerometers, gyroscopes, magnetometers, barometers, and altimeters. One or more of these sensors can be present in an inertial measurement unit (IMU). The motion sensor system can also quantify the degree of motion based on variance in the detector signal from the optical sensor system. Other systems for motion assessment include optical or image detection systems. The motion sensor system can use a singular source of information of motion assessment or combine information from sensors as needed.

A trigger system, e.g., button 1910, is configured to detect a trigger (e.g., pressing of the button) and then initiate a hydration measurement. In other embodiments, the trigger system can be configured to detect a sensor-based, user-based, or time-based trigger. A sensor-based trigger refers to initiation of a hydration measurement based on sensor signals. For example, little or no motion (as sensed by the optical sensor system) or the detection of large pulsatile signals (as sensed by the optical sensor system) can indicate the presence of suitable measurement conditions and can constitute a triggering event in isolation or combination. User-based triggers refer to the initiation of a hydration measurement based on any intentional activity generated by the user. Examples include both activities with the apparatus itself or with an external device in communication with the apparatus. Direct interaction with the apparatus can include a tap, turn, or twist of the device in a defined manner, or a defined hand or finger gesture. In such cases, the trigger system would be configured to be responsive to the motion sensor system. Alternatively or in addition, the trigger system could be configured to be responsive to a user input system, defined below. For example, users can interact with an application on a smartphone to initiate a hydration assessment. A further example can include a triggering event based on voice commands or a defined sound sequence. Lastly, time-based triggers refer the initiation of a hydration measurement based on absolute or relative timing. Such triggers include the elapsed time since the last hydration measurement (e.g., 30 minutes since the last successful measurement), a specific time of day (e.g., 6:00 AM and 10:00 PM every day), or times dictated by or by the user's circadian rhythms (e.g., after the user falls asleep or gets out of bed).

An optical sampling control system (1911) is used to establish and change the operational parameters of the optical sensor system. Operational parameters include parameters of the optical sensor system that can be configured at the initiation of sampling, to include emitter and detector selection, wavelength selection, sampling frequency, detector integration time, ambient light cancellation, and the duration of sampling. During a hydration measurement, when detection of aortic valve opening and closing is required, the following operational parameters for the optical sensor system shown in Error! Reference source not found. in can be suitable: use of both emitters (1905 and 1906) with a near infrared wavelength (e.g., peak wavelength of 940 nm) at near maximal intensity (e.g., drive current of 60 mA); use of detector 1908 to encourage a long optical path with deep sampling of arteries and arterioles, with a maximal integration time of 100 μs; sampling frequency of greater than 100 Hz; acquisition duration of 30 seconds. The above operational parameters are provided for illustration only; variations of these parameters can also be suitable. When a hydration measurement is not taking place, the optical sampling control system can specify a different set of operational parameters. For example, if only heart rate is being determined, the operational parameters can be altered to reduce power requirements and conserve battery life. Such operational parameters can include: use of a single emitter 1906 emitting green light (e.g., peak wavelength of 530 nm) at sub-maximal intensity (e.g., 15 mA); use of detector 1907 to encourage a short optical path where photons largely interact with the capillary bed; sampling frequency of 16 Hz. During periods where no optical measurements are required, the optical sampling control system can also fully inactivate the optical sensor system (achieving an effective sampling rate of 0 Hz for all detectors and drive current of 0 mA for all emitters) to further conserve power.

An analysis system (1912) receives signals from one or more detectors in the optical sensor system and determines the ejection time and inter beat interval. The analysis system can combine or otherwise aggregate signals from one or more detectors and from one or more wavelengths. The analysis system can also use signals from the motion sensor system, e.g., accelerometers and/or gyroscopes, which can be used to minimize or eliminate noise in the detector signal caused by motion or other artifacts. In addition, the system can incorporate signals or extracted features from prior sampling periods. The analysis system can employ filtering or signal transformation, noise-cancellation, feature detection, algorithmic processes, probabilistic models, prediction models, or other analytic techniques.

The analysis system can further comprise a signal suitability system (1913), which determines a metric indicative of the suitability of the acquired signals for hydration determination such that a reliable result will be generated. The determination of suitability can be based on a variety of factors, to include the stability and consistency of the raw or processed detector signals, the consistency or model-based likelihood of extracted features such as ET and IBI, the magnitude of motion as determined with the motion sensor system, the estimated degree of motion contamination in the detector signals. The signal stability system can use outlier detection methods, anomaly detection methods, probability models, or other techniques to assess suitability. The signal suitability system can be configured to determine the cause for a lack of signal suitability and provide this diagnostic information to the user via a feedback system such that corrective action might be taken. Additionally, the signal suitability system can be configured to provide information to the optical sampling control system such that changes in operational parameters can be implemented to improve the quality of acquired signals.

A posture determination system (1914) is a system for determining the body posture of the user. In the illustrative embodiment of Error! Reference source not found. , the posture determination system is configured to be responsive to the motion sensor system 1909. In general, a number of technologies can be used to perform posture determination, but approaches can be grossly divided into (1) direct measurements, (2) inferred measurements and (3) self-reported determination. Direct measurements to determine posture include data from sensors that enable detection of body movement, such as accelerometers, gyroscopes, magnetometers, and cameras or other imaging platforms. These sensors can be worn on the user, or can observe the user from an unattached position, or a combination thereof. Inferred measurements to determine posture rely on the current activities of the user. For example, when sleeping it is reasonable to assume that the user is in a supine or semi-recumbent position. When traveling at a significant speed (e.g., >30 mph) one can infer a seated position in a car or other vehicle. In some embodiments, self-reported measurements of posture determination include the user reporting their posture using a user input system (1917).

A hydration determination system (1915) takes the IBI, ET, and potentially additional information, such as user input, to determine the hydration status of the user. The resulting information can be communicated to the user via a feedback system (1901).

A feedback system comprising display LEDs 1901 provides feedback to one or more recipients. The recipient can include the user and/or an interested party or parties, such as coaches, teammates, caregivers, or medical professionals interested in the hydration status of the user. Feedback as used herein refers to the transfer of any information related to hydration status or a hydration measurement. For example, the feedback can communicate the hydration status of the user, the quality of signals acquired during a hydration measurement, or instructions for making a measurement or taking corrective actions. Feedback on hydration status can be provided to in real or near real-time, allowing the recipient to make near-term lifestyle, fluid consumption, activity, or medication changes to improve performance, recovery, health status, and general wellbeing. In alternative embodiments, feedback can be provided on the device itself, or on an external device, such as a smartphone, cyclometer, smart waterbottle, smart watch or personal computer in communication with the device. Feedback can be visual (e.g., via a readable display or LEDs), audible (e.g., beeps, tonal patterns or speech), tactile (e.g., produced by vibratory or haptic technologies), in the form of an action (e.g., the lid of a smart water bottle popping open) or any combination thereof.

A user input system, Bluetooth receiver 1917, allows information to be transferred from the user to the device. In other embodiments, the user input system can be configured to receive input in many forms to include physical interaction, voice interaction, gesture interaction and other communication methods. For example, to receive physical input, the user input system can comprise a button or switch. To receive gesture input, the user input system can comprise a gesture detection system responsive to the motion sensor system. Gestures can include tapping on the device, rotating the device, or a motion sequence such as clapping the user's hands three times. Additional examples of communication methods include wireless transmission with electromagnetic or ultrasonic waves, wherein the user input system would comprise an appropriate receiver.

The process of obtaining a useful hydration measurement involves coordination and dependencies between various systems. Error! Reference source not found. depicts the operational relationships between the described systems in the illustrative embodiment of Error! Reference source not found. Alternative embodiments can include a subset of interactions, additional interactions, other otherwise differentiated dependencies.

### Mechanisms for Decreasing Transmural Pressure

The apparatus can be configured with mechanisms that change the transmural pressure at the sampling site. To facilitate effective description of these mechanisms and their operation, Error! Reference source not found. defines a coordinate system based on a finger on which the apparatus is worn. The longitudinal dimension is aligned with the length of the finger, and specifically a phalange. An orthogonal radial dimension is defined from the center of the finger to the skin surface. A circumferential dimension, perpendicular to both the longitudinal and radial axes, is defined around the circumference of the finger. Analogous coordinate systems can be defined for other body members. For example, the longitudinal dimension for the wrist is aligned with the length of the ulna and radius, and for the upper arm is aligned with the humerus bone.

The apparatus can be configured to decrease transmural pressure by applying external pressure to the sampling site or in adjacency to the sampling site. Because fingers and other body members are semi-rigid objects with limited deformation capabilities, pressure applied to one location is transmitted throughout the volume of tissue with reasonable efficiency. For the finger, an adjacent area is considered to be within a given phalange. External pressure can be exerted locally, circumferentially, at a single longitudinal location or distributed along the longitudinal axis.

The apparatus can be configured to decrease transmural pressure by decreasing the effective internal diameter of an internal surface that surrounds all or a significant portion of a finger or other body member. The effective internal diameter is defined as the largest circle that can be inscribed by the internal surface of the apparatus, as viewed from a longitudinal projection. Error! Reference source not found. illustrates the determination of effective internal diameter for a number of different ring-type forms that can partially or fully encircle a body member. In Error! Reference source not found. A-F, thick black lines denote the internal surface of the ring; dashed lines denote the largest inscribed circle and corresponding diameter. Error! Reference source not found. F shows the effective internal diameter for a ring with an open form (i.e., does not completely encircle or enclose the body member).

For the purposes of illustration, several different mechanisms for changing effective internal diameter will be discussed, as incorporated into a finger ring; the majority of these mechanisms are equally applicable to devices worn around the wrist, upper arm, or other body members. For purposes of explanation, configurations that exert less pressure at the sampling site will be referred to as the “worn state”, whereas configuration exerting greater pressure (and thus reducing transmural pressure) will be referred to as the “measurement state”.

One means for changing effective internal diameter is through a gross change in the inner circumference of the ring, similar to tightening or loosening a belt. Embodiments of this type are considered to have a reducible internal circumference. The illustrative embodiment in Error! Reference source not found. includes such a means for changing the effective internal diameter: an integrated ratchet mechanism 1903 that can tighten the inner flexible surface of the ring 1921 during a measurement state, and loosen it during the worn state. Error! Reference source not found. A-B shows a second example of a gross reduction in inner circumference. This example embodiment shows an open form ring capable of flexure when force is applied. In the worn state (Error! Reference source not found. A), the ring form remains open. To achieve the measurement state (Error! Reference source not found. B), the user applies force to the sides of the ring (across the opening) to compress the ring to the defined stop location. A trigger system can be configured to sense when contacts, 2301, at the open ends of the ring meet, and the initiate a hydration measurement. This process constitutes a user-based trigger, as well as a trigger based on the detection of a change in effective internal diameter. A signal suitability system can require persistent contact throughout signal acquisition as criteria for signal suitability. Additional examples of embodiments configured for gross circumferential change rings are shown in Error! Reference source not found. C-E. Error! Reference source not found. C is similar to the device of Error! Reference source not found. A in that application of force by the user reduces the circumference and compresses the ring to a defined stop. Error! Reference source not found. D also changes from an open form to a closed form, and incorporates a clip mechanism to maintain the reduced circumference configuration in the measurement state such that the user is not required to apply continual force throughout measurement period. Error! Reference source not found. E incorporates a screw or “roller” mechanism that gradually changes the circumference, and thus creates the ability to incrementally increase or decrease transmural pressure.

An alternative means for changing effective internal diameter is via the movement of one or more compressive features into the interior of the ring. Error! Reference source not found. illustrates an example of an embodiment with a singular compressive feature, asymmetric block 2501, mounted on the top of the ring. In the worn state (Error! Reference source not found. A-B), the feature is rotated such that it does not compress the finger. To enter the measurement state (Error! Reference source not found. C) the user rotates the compressive features by 90 degrees where it locks into a stable configuration. The rotation of the feature creates a significant change in the effective internal diameter. Error! Reference source not found. shows drawings of additional types of compressive feature rings. In all cases there is a mechanism for creating a protrusion within the interior of the ring via local movement. Embodiment Error! Reference source not found. A operates via a force in the radial dimension, which moves compressive feature toward the interior of the ring. Embodiment Error! Reference source not found. B operates by squeezing the ring with opposing forces in the radial dimension, forcing a flexible element to “bump up” into the interior of the ring. Error! Reference source not found. C operates via a latch, which when “flipped up” rotates a compressive into the interior of the ring.

Alternative embodiments constitute protuberance rings, which comprise a multitude of protuberances on the internal surface of the ring that change configuration. The protuberances can be connected together or act independently to change the effective internal diameter. Error! Reference source not found. is an illustration of a protuberance ring in operation. In the wear state, the material between the ring and the finger is in a “down” state and fills a smaller volume between the ring and the finger, 2601. The material between the ring and finger is a material composed of many stiff protuberances, 2602, on a flexible supporting layer, 2603. When the ring has been rotated in a clockwise direction, the protuberances are angled toward the supporting layer and the effective internal diameter is smaller, 2604. In the measurement state, the material between the ring and finger is in an “up” state, and fills an enlarged volume state between the ring and the finger, 2605 (material enlargement exaugurated in size for clarity). When the ring is rotated in the counterclockwise direction, the protuberances are rotated or moved into a more vertical position, 2606, resulting in decreased effective internal diameter, 2607. Protuberance type materials that exhibit this type of behavior are frictional anisotropy-based systems, mohair cross country climbing skins, brushes where the “comb elements” are moderately rigid but are mounted in a material that is flexible, internally architectured materials, parallel ribs, gills, rotational blinds, and 3D printed structures to include hair-like elements. The stress and force interactions of the system have been modeled and several types of structures illustrated by Bafekrpour et. Al. Bafekrpour, Ehsan, et al. “Internally architectured materials with directionally asymmetric friction.” Scientific reports 5 (2015): 10732. The protuberances can be composed of single filaments, a small block, or “pleats or blinds”. If all protuberances are linked together, the movement of one can encourage the movement of other protuberances. To facilitate this coordinated conformational change, material may be placed between the protuberances so as to create a linked interaction between protuberance members. Additionally, the material between the protuberances or the attachment of the protuberances to the supporting layer may impart a configuration bias in the native or resting location of the protuberances. The material between the protuberances can be used to create a linked interaction between protuberance members or to impact a bias in the native or resting location of the protuberances. Error! Reference source not found. is an example embodiment of a protuberance ring where only the upper half of the ring has protuberances. The illustrated ring shows an inner surface connecting the protuberances. As shown in Error! Reference source not found. , counterclockwise rotation of the ring causes a configuration change and a reduction in the internal diameter. The counter-clockwise rotation of the ring results in a sight compression of inner surface, 2702, as the protuberance structure becomes more vertical, 2703. This compression creates an inherent mechanical bias that returns the structure to the wear state. The figure illustrates a connected structure but many variances in implementation exist to include independent members, parallelograms, or other geometric shapes, 2701. Many variances of the protuberance ring concept exist to include different material types, the ability to lock the ring into a measurement mode, and protuberance structures that have asymmetric bend profiles. Depending on the details of implementation, the protuberance ring concept can be implemented as the movement of one or more compressive features into the interior of the ring, as shown in Error! Reference source not found. However, if the protuberance structure has a singular inner surface, then the result is a gross change in the inner circumference of the ring.

Another approach for changing the effective internal diameter is through the movement of ring features along the longitudinal axis. Error! Reference source not found. shows an example of device configured with such an approach. In the worn state configuration (Error! Reference source not found. A), the features comprising the terminal ends of the ring, 2801, abut each other in the same cross-sectional plane. The device can have mechanisms to maintain or stabilize this wear configuration, not shown. The application of opposing forces, 2802, in the longitudinal dimension causes a conformational change resulting in the measurement state configuration (Error! Reference source not found. B). In this conformation, the ends of the ring, 2801, by-pass each other in the longitudinal dimension and are no longer in the same cross-sectional plane. The resulting change in configuration decreases the effective internal diameter, and decreases the transmural pressure at the sampling site through the application of force at or in adjacency to the sampling region. The “measurement state” conformation represents the native or low stress configuration, thus the device maintains this state throughout the measurement. The ring is designed such that the inherent mechanical bias of the system is to maintain a stable measurement state. Error! Reference source not found. presents a second example of a ring embodiment configured to reduce the effective internal diameter through the movement of features along the longitudinal axis. In the worn state (Error! Reference source not found. A), features comprising wire loops, 2901, are positioned parallel with the body of the ring. In this configuration the effective internal diameter is maximal. Via a release mechanism (not shown), the wire loops pivot away from the ring in the longitudinal dimension, resulting in a reduction of the effective internal diameter change for the measurement state (Error! Reference source not found. B). The mechanism for force in this example embodiment is generated by the wire loops: the lengthwise ends of each wire loop are oppositely coupled to the ring at a coupling point, 2902, such that that the spring/rebound rigidity of the loop creates an automatic biasing mechanism. As the wire loops pivot about the coupling point, the torsional properties of the wire loop generate a biasing force that mechanically urges the loop toward the open or expanded configuration. This inherent mechanical bias creates a stable measurement configuration. The wire loops exert force on the top of the finger, which is opposed by internal surface on the bottom of the ring, effecting a reduction in transmural pressure at the sampling region.

An additional ring embodiment capable of creating change in internal effective diameter is the respective movement of two or more rigid bodies. These rings systems can create changes in effective internal diameter via both rotational and translational movement of two rings or other geometric shapes relative to another. Error! Reference source not found. illustrates the concept in both rotation and translation. In the rotational design, the rings rotate relative to one another, but the axis of rotation is not the same. For example, the dashed line ring rotates about point 3001. Due the non-symmetrical rotation point, the rotation leads to a change in the effective internal diameter, as show via the cross hatched area. Similar changes can be made by translating one ring or geometric shape relative to the other. In the wear configuration, the rings are concentrically located and the internal effective diameter is maximal. As one ring translate relative to the other, the effective internal diameter decreases as shown by the cross hatched area.

The above embodiments are illustrative in nature and do not represent all physical mechanisms for producing a decrease in transmural pressure. One of ordinary skill could develop multiple variants or alternatives based on the goal to change the effective internal diameter.

### Stability of Measurement State

As illustrated and subsequently described, several ring embodiments require the user of the ring to maintain a force or pressure during the measurement. Examples of such user-dependent maintenance-force embodiments include those rings in illustrated in the following figures: 23 A-B, 23C, 25B, and 27. Other embodiments include a physical mechanism to stabilize the measurement state configuration, for example a latch or retention component: 23D 25A, and 25C. A stable measurement configuration can also be achieved via ratchet mechanisms, 19 or a screw mechanism, 23E. Error! Reference source not found. illustrates stable condition due to the shape of the rotated element. Error! Reference source not found. and Error! Reference source not found. represent embodiments where the inherent mechanical bias of the system creates a stable measurement state resulting in decreased transmural pressure. Ring embodiments that have a stable measurement state can be preferred by the user as they do not require the engagement of operator during the measurement period. Additionally, these rings will generate measurement data that is not subject to movement of the ring due to the use of the other hand.

### Example Methods for Hydration Assessment

The method of operation is a significant element of the invention; key information should be acquired and criteria fulfilled in the anticipated use environment, where individuals will perform the activities of daily and can engage in exercise and athletic events. Increases in physical activity will result in increased heart rate, skin contaminations, and peripheral vasodilation. The hydration assessment method of the current invention is robust to these expected conditions and potential error sources. The method of operation and associated systems make this difficult measurement by the judicious balancing of error source minimization, signal optimization, physiological signal manipulation, and feedback to the user.

### Process for Hydration Determination

The process of obtaining a hydration measurement involves a series of steps that provide robust and reliable device performance. FIG. 31 is an example embodiment of such a process. The process for a hydration measurement is initiated by a trigger event that can be sensor-based, user-based, or time-based. Following the detection of such a trigger event, the hydration measurement process is initiated. The optical sampling control system provides a set of operational parameters to the optical sensor system. The optical sensor system, and in some embodiments the motion sensor system, then acquire signals for the designated measurement period. Following signal acquisition, the sensor signals are analyzed by the analysis system, and concurrently or subsequently analyzed for suitability by the signal suitability system. If the signal is not determined to be adequate, feedback is provided to a recipient via the feedback system. If the signal is determined to be adequate, information extracted by the analysis system, and potentially other systems, is provided to the hydration determination system. A hydration status is determined, and this information is provided to the recipient. Following this step, the device can enter an “idle” state in “wear mode” with a modified set of functions (e.g., assessment of user motions and heart rate) while waiting for another trigger event to be detected.

### Triggering a Hydration Measurement

An example of a hydration measurement trigger sub-process is illustrated in FIG. 32. The disclosed embodiment contemplates a variety of triggering methods. In this example process, hydration measurements are triggered only when requested by the user (by means of a specific gesture, interaction with a connected device, or physical deformation of the ring), or if a significant time interval has passed since the last measurement. In this example embodiment, feedback is provided to the user regarding hydration determination status: if a measurement process is triggered, indicator LEDs blink green in succession. In alternative embodiments feedback can take other forms. This example trigger sub-process supports battery conservation, a vital objective for small wearable systems. Hydration measurements are performed only every 30 minutes unless specifically triggered by the user. Practically speaking, there is little need for more frequent hydration determinations since changes in systemic hydration are relatively slow, except when internal heat generation or external heat creates rapid change that can be in the liters of sweat per hour.

### Alternative Process for Hydration Determination

FIG. 33 presents an alternative process for hydration determination that includes coordinated interaction between systems, power management, and user involvement to achieve a reliable result. As in FIG. 31, following a trigger event, the optical sampling control system designates a set of operational parameters to the optical sensor system and signals are acquired from one or more sensor systems. Deviations from FIG. 31 begin after the evaluation of signals for suitability. If the suitability metric indicates that the signals are inadequate, then the optical sampling control system can change the operational parameters, and the signal acquisition/analysis steps are repeated.

In cases where signal inadequacy is due to failed or unreliable detection of aortic valve opening or closing, changes in the optical system operational parameters can achieve signal suitability. As an example, high-fidelity sampling defines a class of operational parameters that supports the detection of both the aortic valve opening and aortic valve closing events and comprises any combination of increased sampling rate, increased light intensity, increased detector integration time, and increased sample averaging. These increases are defined relative to operational parameters used in standard-fidelity sampling that enables only detection of aortic valve opening or related signals, i.e., heart rate determination. The use of high-fidelity sampling comes at an expense, as it consumes far more power than that required by standard-fidelity sampling. The additional power consumption creates a challenge for wearable devices with limited battery sizes and a mandate for power conservation to prolong battery life. Thus, embodiments of the invention can employ high-fidelity sampling only as necessary and in a staged manner to prolong battery life to the extent possible.

In some embodiments, changes to operational parameters can also include changes in emitter wavelength and the set of active emitters and detectors to affect the optical path and the vascular structures with which photons interact. Shorter wavelengths in the visible range and selection of proximal emitters and detectors encourages reflection dominant sampling, i.e., optical sampling of the tissue where the majority of photons do not penetrate deeply into the tissue and primarily interact with (i.e., are reflected by, scattered by, or absorbed by) vessels in the capillary bed. Longer wavelengths in the near infrared range and selection of emitters and detectors with greater physical separation (>5 mm) encourages transmission dominant sampling, i.e., optical sampling of the tissue where the majority of photons penetrate and travel through the tissue, interacting with (i.e., reflected by, scattered by, or absorbed by) tri-layered vessels. Though detection of aortic valve closure is typically aided by transmission dominant sampling, reflection dominant sampling can be preferred during with greater user motion since reflection dominant signals are less likely to be contaminated by venous sources. Thus, an example embodiment can first acquire signals with a set of operational parameters consistent with transmission dominant sampling, then depending on signal suitability, acquire additional signals with a second set of operational parameters consistent with reflection dominant sampling.

If a suitable signal has not been acquired and operational parameters are at a maximal level (e.g., LED drive current cannot be safely increased), or the power consumption or battery usage exceed defined thresholds, then additional steps can be taken. As illustrated in FIG. 33, decreases in transmural pressure can be used to improve signal quality. Transmural pressure decreases can be achieved with a variety of processes, to include (1) raising the sampling region relative to the level of the heart, (2) manually pushing the device against the sampling region, (3) moving the device to another sampling region (such as a region of the same finger with a larger circumference, or a different finger with a larger circumference) such that greater external pressure is applied, or (4) reducing the effective internal diameter of the device such that greater external pressure is applied to the sampling region. These processes can be used alone or in combination to create the desired effect. As these changes require the active participation of the user, feedback to the user is provided. As signals are acquired and analyzed, the signal suitability system can continuously access whether the decreases in transmural pressure have created the desired effect of suitable signals. In some embodiments, the system can modify operational parameters concurrently, in parallel, or in sequence with changes in transmural pressure to achieve suitable signal with the minimal power expenditure. Depending on the mechanism for reducing transmural pressure, the changes can be incremental (e.g., gradually raising a finger on which the device is worn, or slowly reducing the effective internal diameter using an embodiment of the device like that illustrated in FIG. 23E) or discrete (e.g., moving the device to another finger region or using an embodiment of the device like that illustrated in FIG. 28). The desired extent of transmural pressure decrease is limited: decreasing the arterial transmural pressure below zero at diastole by any mechanism or combination of mechanisms will begin to occlude arterial flow and distort the pulse waveform. Additional decreases in transmural pressure beyond this point will not improve the signal, and might render the signal unsuitable for hydration determination. As shown in the example process in FIG. 33, feedback is provided to the user if the maximum achievable reduction in transmural pressure change has been produced and the signal remains unsuitable for hydration determination.

### Motion-Scenario-Opportunity Sampling

The device can incorporate Motion-Scenario-Opportunity Sampling (MSO Sampling) to “seize the moment” for high-quality data acquisition. While the user is engaged in motion-intensive activities, the operational-control system continuously scans for micro-windows that satisfy the prescribed measurement cadence. During a basketball game, active play introduces substantial motion artefact, yet a free-throw pause, time-out, or change of possession provides a brief low-motion interval that is ideal for measurement. Similarly, during high-intensity-interval training (HIIT), each sprint phase is followed by a short recovery period in which body movement subsides and heart rate decelerates; MSO Sampling targets these quieter recovery intervals, capturing clean plethysmographic waveforms without disrupting the workout. When an opportunity arises, the device opens a sampling window, temporarily adjusting optical drive current, detector gain, or sampling rate only for the duration required, then reverts to its power-efficient baseline. This context-aware strategy focuses resources on measurements with the highest likelihood of success, avoids energy-wasting attempts during heavy motion, and helps extend battery life.

### Situational Awareness System

In contrast to conventional “one-rate-fits-all” wearables, the present invention describes a sensor system that actively interprets its situation to measure hydration effectively. The system continuously fuses real-time thermoregulatory signals, such as heart rate, heart rate variability, motion intensity, activity intensity and duration, and respiration rate with environmental heat-load indicators such as ambient temperature, relative humidity, solar radiation, altitude, and geolocation. It converts these measures into a composite probability of hydration change risk score that enables real-time adjustments of measurement cadence and operational parameters, so device operation adapts to ensure the capture of hydration measurements that engage effective hydration management. The adaptive concept comprises: when a user's hydration is essentially stable, low measurement cadence hydration measurements are sufficient; but when exercise, fever, or hot environments accelerate fluid loss, the device should increase the measurement cadence or frequency so that changes of roughly 1% TBW or 0.5% total-body water are detected in near real-time. This situationally aware adaptive process uses the control logic to score the situational inputs continuously and, whenever the score exceeds a threshold, the system adaptively increases the hydration-measurement cadence. When the likelihood of hydration change diminishes, the control logic can decrease the measurement cadence, conserving battery life without sacrificing clinically meaningful fidelity. By linking situational awareness directly to adaptive control of measurement frequency, the invention delivers hydration tracking that mirrors the dynamics of fluid loss while maximizing run time, thereby resolving the longstanding trade-off between measurement precision and power efficiency in wearable hydration monitors.

### Predictive Assessment System

The situationally aware system fuses real-time thermoregulatory signals (heart rate, skin temperature, activity level, respiratory rate) with environmental factors (temperature, humidity, altitude, location) to anticipate periods when hydration is likely to change. Because unexpected influences—such as the initiation of a diuretic, an acute gastrointestinal illness, or a rapid shift in cabin pressure—can still provoke sudden fluid loss, the predictive assessment system continuously re-estimates the instantaneous hydration slope (dH/dt) based on prior measurements, quantifies its confidence, and tightens or relaxes the measurement cadence so that unobserved deviation in hydration never exceeds a predefined threshold. This predictive assessment system uses prior measurement data to estimate the anticipated change in the future so effective hydration management can occur.

The predictive assessment system defines an adaptive hydration-monitoring methodology that replaces fixed-interval sampling with a forecast-driven cadence grounded in measured hydration dynamics. After each non-invasive measurement of effective circulating volume (or an equivalent proxy for total body water), the assessment system derives a short-term rate of hydration change, dH/dt, from a rolling window of prior readings using a weighted-least-squares or Kalman-filter estimator. That derivative—and its quantified uncertainty—feeds a predictive model that projects the user's hydration trajectory forward in time. The system then selects the earliest future instant τ (a time period) at which the projected absolute change equals a preset threshold ΔHₘ_{^{ax}} (e.g., 0.5% TBW), subject to safety bounds on minimum and maximum interval length. By guaranteeing that unobserved hydration deviation never exceeds ΔHₘ_{^{ax}}, the system automatically compresses the measurement cadence intervals during periods of rapid fluid loss and elongates them during quiescent states, thereby reducing energy consumption and data burden without compromising clinical fidelity.

The predictive assessment system can define a period of time where the projected change equals a defined threshold, provide a probability measure or other numerical or categorical value that provide insight of the projects rate of hydration change.

### Combined Hydration Sensor

Estimating the probability of a hydration change is not a single, hard-wired equation; it is a flexible decision-making framework that can be implemented in many ways. In one embodiment, the invention features a dual-layer architecture that unifies inference and measurement to manage hydration surveillance. A Situationally Aware System performs an indirect assessment of fluid status by synthesizing contextual cues—such as ambient climate, physical activity, altitude, and heart rate—whose collective behavior is statistically associated with dehydration yet does not constitute a direct measurement. Operating in parallel, a Predictive Adaptive System forms a data-driven projection by analyzing historical sensor readings of effective circulating volume, total-body water, or a comparable biomarker, thereby deriving an empirical rate of change. Either layer, acting independently or in concert, may indicate that projected fluid drift could exceed a tolerance threshold; in response, the overarching control logic adaptively adjusts the timing of the next measurement. By partitioning inference based on ancillary information from prediction grounded in actual hydration measurements, the invention preserves implementation flexibility—permitting a wide range of sensing modalities, statistical techniques, and control strategies-while ensuring timely detection of both expected and unforeseen hydration perturbations.

### Data Acquisition

A hydration measurement begins when the measurement-cadence system issues a “sample now” request. The operational-control system fulfils that request by collecting plethysmographic data at a baseline rate of at least 100 Hz. During or immediately after capture, the measurement-suitability system applies a multi-metric quality-control (QC) evaluation of signal-to-noise ratio, detector saturation, motion-artefact indices, concordance of LVET and heart-rate features, and overall waveform morphology. If the plethysmographic data fails the QC process, the measurement is rejected and the operational-control system modifies one or more operational parameters before initiating a new data acquisition.

People become dehydrated by many pathways, but two dominate: internally generated heat from sustained muscular work and externally imposed heat from a harsh environment. Both pathways are associated with increased body motion, elevated heart rate, possible degradations in pulsatile amplitude, and expose of the optical system to bright sunlight, each a known antagonist of clean data acquisition. To counter these disturbances, the operational-control system runs an activity-recognition system that fuses motion patterns, geolocation, ambient conditions, and historical user behavior to infer what the wearer is doing at any moment and to adapt the sampling strategy accordingly.

The following is an illustrative example of how such a system could operate with four example modes of activity and a recommended operational parameter change.

Short-burst activities—When the activity-recognition engine detects sports characterized by brief spikes of motion followed by natural pauses—such as golf or baseball—the operational-control system leans on MSO Sampling. It waits for the lull between swings, pitches, or “live-ball” periods to open a sampling window. Because these sports often unfold under full sun, the system may also switch to an optical configuration that shields the detector from direct sunlight (e.g., using a finger- or glove-shadowed photodiode) to prevent saturation and preserve signal quality.

Mixed-burst activities—Sports like soccer, hockey, and basketball present alternating stretches of intense movement and brief stoppages. Upon recognizing this pattern, the operational-control system couples MSO Sampling with a transient boost in sampling frequency during the next lull, ensuring that left-ventricular-ejection-time (LVET) resolution is maintained despite elevated heart rates. If ambient lighting varies—stadium floodlights versus shaded sideline—the optical-acquisition profile is adjusted dynamically so the ensuing waveform still satisfies the QC review.

Sustained high-motion activities—Continuous-motion pursuits such as road cycling or distance running provide few opportunities for MSO sampling. In these scenarios, the operational-control system temporarily may recommend a change in transmural pressure. For example, the ring as illustrated in FIG. 28, could be used in the reduced diameter mode during the activity. The resulting change in transmural pressure will increase the size of the arterial signal and decrease artifacts associated with venous blood movement. The increased sensor-skin pressure reduces vibrational artifacts and enable data capture without waiting for a pause in activity that may not come for several hours. Emitter power can be elevated to help compensate for motion artifacts caused rhythmic handlebar or foot-strike vibration.

Extreme environmental activities—Surfing and white-water kayaking impose optical challenges: intense reflected light, intermittent water between sensor and skin, and cold water induced arterial vasoconstriction. When such conditions are recognized, the operational-control system may change the optical acquisition profile. Profile change may include the use of a longer pathlength configuration, with greater LED drive current, and the use of multiple detectors. MSO Sampling may also be used during transient stillness.

Closed-loop assurance—Through this continuous cycle of capture→quality-check→classify→adjust→re-capture, every hydration estimate is anchored in plethysmographic data that meet predefined fidelity criteria, regardless of whether dehydration risk stems from internally generated heat, externally imposed heat, or a combination of both. By tailoring its response to the recognized activity class and prevailing environmental context, the device delivers reliable measurements while consuming only the energy necessary for success.

### Example Embodiment

An example embodiment for illustrative purposes is illustrated in FIG. 41. The Measurement Subsystem (4101) contains the sensors that measure the development on internal head load and external heat load. The sensor package may include a measurement system that enables the determination of heart rate, skin temperature, external air temperature, respiratory rate, and motion. The motion measurement system is likely a inertial-measurement unit (IMU). An IMU is a compact sensor package that typically combines tri-axial accelerometers to measure linear acceleration and tri-axial gyroscopes to measure angular velocity; many modern IMUs also add a tri-axial magnetometer for heading and sometimes a barometric pressure sensor for altitude estimation. Together, these channels let the device track motion, orientation, and position changes of the host platform in real time. Location, time and other ancillary information can be obtained vai an active connection to the internet. Other measured values may include a core-temperature surrogate, skin conductance/galvanic skin response, local ambient humidity, solar-radiation/UV index, barometric pressure/altitude, photoplethysmography-derived SpO₂, heart-rate variability, bioimpedance spectroscopy, optical/microfluidic sweat-rate sensor, exhaled CO₂/tidal-volume proxy, skin-blood perfusion index, lactate micro-sensor, glucose micro-sensor, ambient wind speed, and acoustic breath-sound/cadence microphone.

These measured values or any associated subset can be provided to the Probability of Hydration Change System which converts them into an estimate of sweat loss, some useful surrogate, or probability. An estimation of sweat loss will be used for illustrative purposes. Sweat loses via internal heat can be estimated as follows. The system tracks the rise in heart rate above the user-specific baseline and applies the ISO 8996 heart-rate method to translate that deviation into real-time metabolic power. That metabolic rate—together with personal factors such as height, weight, sex, and age—is passed to an empirically validated whole-body equation, an example of such an equation can be found in Jay et al., “Whole-body sweat-rate prediction: indoor treadmill and cycle-ergometer exercise,” J. Appl. Physiol., 137 (4): 1014-1020, 2024. The result of these equations and calculations is an instantaneous estimate of milliliters of sweat produced per minute.

Environmental heat load can be quantified independently. Ambient temperature, relative humidity, and other potential parameters such as wind speed, and altitude can be used. Temperature, relative humidity, and wind speed, measured on-device or fetched via geolocated weather data are supplied to the ISO 7933 Predicted Heat Strain (PHS) model with other assumptions, which returns the additional evaporative heat flux (and hence sweat rate) required to maintain thermal balance under the current user conditions.

Probability-of-Hydration-Change System is an anticipatory engine that combines situational inputs, internal-heat proxies (heart-rate elevation, respiration rate, distal-skin temperature), external-heat indices (ambient temperature, humidity, solar load, wind), and contextual markers (altitude, time of day, geolocation), to forecast imminent fluid loss. For every measurement cycle it emits two numbers: (i) a 0-1 probability that net fluid loss will exceed the clinician-defined threshold within the next cadence window, and (ii) the expected change-rate in % TBW per hour. Because it draws on continuously streaming situational data, this engine can refresh its outputs multiple times per minute, independent of the hydration-measurement cadence.

Predictive-Assessment System operates on the chronological series of completed hydration measurements; this retrospective engine fits a short-horizon trend to the most recent data points. After each new measurement, it emits the same two outputs as its anticipatory counterpart: (i) a 0-1 probability that hydration status will exceed the clinician-defined threshold before the next scheduled reading, and (ii) the expected rate of change in % TBW per hour. Because it updates only when a new hydration measurement is taken, its refresh rate is limited by the current measurement cadence.

Cadence-Control System can ingest the two probability scores and two rate-of-change estimates produced by the upstream systems, harmonize their differing update frequencies (fast-streaming situational forecasts versus cadence-bound trend estimates), and set the next measurement interval so that the maximum projected % TBW change within that window remains below the clinical limit. If both engines signal low risk, cadence may be decreased or remain at an energy-saving baseline; escalating risk shortens the interval so that additional data are captured before the threshold is reached. The cadence control system issues a “take data trigger” to the Operational-Control system at the defined measurement cadence.

Operational-Control System (OCS) is the integrated hardware, firmware, and software layer that executes real-time adjustments to sensor “operational parameters” (e.g., sampling frequency, optical acquisition profile, sensor-to-skin transmural pressure) whenever such changes are required by power-management policy or by data-quality feedback from the Measurement Suitability System. Once the Cadence control system triggers a measurement, the OCS starts the acquisition sequence, monitors the suitability flag returned by the Measurement Suitability System (MSS), and, if that flag is negative, iteratively retunes one or more operational parameters or invokes Motion-Scenario-Opportunity (MSO) sampling to capture a replacement data set of plethysmographic data. This closed-loop system takes data, evaluates, and refines operational parameters to ensure that every hydration estimate is based on plethysmographic data that meet pre-defined fidelity criteria.

Plethysmographic Data Acquisition involves sampling plethysmographic waveforms at or above 100 Hz for about thirty seconds using an optical sensor system configured to detect both aortic-valve opening and closing. After or during data acquisition, the plethysmographic data is sent to the MSS for quality control; only data that is authorized for further physiological analysis is forwarded to the Hydration Determination System.

The Measurement Suitability System (MSS) is illustrated in FIG. 19 represents the analytics subsystem, implemented in hardware, firmware, and software, that evaluates each acquired waveform against quantitative quality metrics, such as signal-to-noise ratio, motion-artifact indices, and confidence in derived features (heart rate, left-ventricular ejection time). It returns a binary or graded suitability flag to the OCS; a negative flag prompts the OCS to modify operational parameters or reschedule the measurement, whereas a positive flag releases the data or subsequent analysis by the Hydration Determination System.

The Hydration-Determination System, as illustrated in FIG. 41, can be implemented in multiple modes and embodiments but performs the following functions: (i) the plethysmographic waveform that has passed the Measurement Suitability System, (ii) baseline user descriptors such as body mass, sex, resting heart-rate profile, and baseline % TBW, and (iii) optional situational tags including ambient temperature and recent activity level. A calibrated prediction model—executing locally on the device's microcontroller or, in alternative embodiments, on a paired smartphone or cloud service—processes these inputs to produce two outputs: (a) the current estimate of total-body-water expressed as % TBW and (b) a unit-less probability that fluid loss will exceed a clinician-defined threshold before the next scheduled measurement. These metrics are passed to the Cadence control system for display, alert generation, and further adjustment of measurement timing.

The Hydration Determination System (HDS) ingests the suitability-approved plethysmographic waveform and returns a current estimate of total-body water (% TBW). In optional embodiments, the HDS may use baseline or historical information, such as earlier measurements from the same subject, body-mass data, or Measurement Subsystem data. The resulting % TBW value is conveyed (i) to the Predictive assessment system for trend analysis and cadence adjustment and (ii) to a user-display or communication subsystem (not shown). The user-display subsystem can assume many forms, including—without limitation—a wrist-worn screen, a paired smartphone application, a voice or acoustical communication, a cycling computer, a web-based dashboard, an electronic-health-record interface or any mechanism the informs any person involved in the hydration management of the user.

### Off-Device Processing

In some embodiments the wearable hydration-monitoring device is responsible only for optical plethysmographic data acquisition, driving the emitters and detector, sampling the waveform, and wirelessly forwarding the resulting data. The on-device electronics may only include plethysmography data acquisition, temporary buffering, and a low-power interface such as Bluetooth LE, Wi-Fi, or other communication systems.

One or more subsequent operations, without limitation, plethysmographic data processing, processing of measurement subsystem information, operational parameter determination, signal-quality assessment, feature extraction (e.g., inter-beat interval, ejection-time interval), hydration estimation, trend analysis, and measurement-cadence scheduling, may be executed on an external processing device such as a smartphone, tablet, personal computer, or cloud server. Which specific functions are performed off-device, and in what order, can vary across implementations without departing from the overall system concept. Hydration results generated off-device can be relayed (i) back to the wearable for haptic or visual notification and/or (ii) to a user-interface element associated with the external processing device, which may take the form of a mobile-app dashboard, smart watch, eyewear, cycling computers, web portal, electronic-health-record integration, or other display mechanism. The wearable and the external processor can employ any suitable wired or wireless protocol.

### Demonstration of System

Hydration Assessment During Exercise-Induced Hypertonic Dehydration. To demonstrate the feasibility of hydration assessment using the present invention, an exercise-induced dehydration protocol was performed. A subject exercised at a moderate exertion level in a heated room (78 deg) without fluid consumption for 60 minutes. The test subject then entered a recovery phase, using oral rehydration to replenish fluids at a consumption rate supported by prior studies with the objective of restoring the subject's weight to the baseline value. Reference and novel measurements of hydration were performed at baseline (pre-exercise), immediately following exercise, and at approximately one-hour increments throughout the recovery phase. Reference hydration measurements included nude weight, urine output, urine specific gravity, and urine color. The novel hydration measurements were made with the current invention. The aortic valve time sequence was acquired using a near-infrared photoplethysmography (PPG) sensor placed at the base of the finger while the subject rested in a single body position (standing).

FIG. 34A shows a schematic of the study protocol, indicating the timing of measurement periods relative to cycling and incremental rehydration. FIG. 34B shows the percent weight change of the subject over time. FIG. 34C shows the amount of fluid consumed in each phase and FIG. 34D shows the change in the color of the urine at each measurement. As seen in many previous studies, urine-derived indices of hydration will lag behind weight due to filling of the bladder. FIG. 34E shows the noninvasive hydration assessments obtained from an embodiment of the invention. IBI and ET were extracted from the PPG measurements and were entered into a linear hydration determination model. Examination of the figure shows a strong relationship between the measurements results and the hydration status of the subject as defined by weight change.

To confirm the specificity of the novel measurements for hydration versus other physiological changes associated with exercise, a second study was conducted. FIG. 35A shows the general experimental design and protocol variants. In Protocol 1, a subject repeated the exercise-induced dehydration as described above. The subject lost considerable fluid resulting in a 4.5% loss of body weight due to fluid loss. On a second day, the subject performed Protocol 2, exercising in identical conditions with matched power output, but consuming fluids during exercise at a rate consistent with the sweat rate. This protocol was designed to minimize hydration changes in the presence of significant physiological changes induced by exercise. Examination of FIG. 35B shows that the subject's weight was maintained during the exercise period via the consumption of nearly 2 L of fluid (FIG. 35C). FIG. 35D shows that in protocol 2, urine was largely unchanged, or perhaps even lighter following exercise. Examination of the results generated with aortic valve opening and closing in FIG. 35E shows a clear distinction between the protocols, with Protocol 2 showing little change or even a slight increase in hydration status following exercise.

The experimental design illustrated in FIG. 35A was repeated in a larger study involving 11 competitive cyclists. Participants completed a standardized cycling protocol in the absence or presence of oral fluid replenishment. Reference hydration status, assessed at ˜1.5 hour intervals during dehydration and subsequent rehydration, was determined from percent weight change and urine specific gravity. When exercising in the absence of fluid replenishment, subjects maximally lost between 2.1 and 3.6% body weight, with a mean of 2.8±0.5% (mean±SD). When exercising in the presence of prescribed rehydration, subjects lost an average of 0.4±0.5%.

Using the inputs and determination model approach outlined in FIG. 17, hydration status was assessed based on noninvasive measurements of IBI and ET. In all subjects, estimated hydration status was significantly reduced at peak dehydration relative to a euhydrated baseline, and a rehydrated recovery period (P<0.0001 in all cases, corrected for multiple comparisons, two-sample t-tests). Incorporating data from all subjects and visits (n=176 observations), binary classifiers were trained to detect 1.5% dehydration. Models achieved greater than 90% accuracy and ROC area under the curve (AUC) values exceeding 0.90 when tested on unseen data, using a 5-fold cross-validation approach.

### Hydration Assessment During Simulated Changes in Isotonic Hydration

An additional demonstration of an example embodiment of the invention was pursued via changes in plasma volume at a fixed body position, without exercise, or associated changes in body temperature. Lower Body Negative Pressure is an experimental approach for inducing decreases in vascular volume and creates a simulated state of isotonic dehydration. The use of lower body negative pressure pulls blood into the lower body and creates transient dehydration via hypovolemia that can be reversed rapidly. FIG. 36 shows the valuable insights available by effectively processing the aortic valve information. The aortic valve time sequence information was recorded using a near-infrared PPG sensor from the tip of the finger. Negative pressures between 0 and 75 mmHg were used, and the pressure was held for approximately 10 minutes at each level. Dashed lines represent the least-squares linear fit to the HR vs ejection time data at each pressure. The “0R” condition indicates the recovery period. The increase in heart rate that occurs between negative pressure changes represents the physiological response to maintain cardiac output. Examination of the figure shows a defined grouping of points with each simulated dehydration level. The plot effectively demonstrates how ejection time and heart rate can be used to define the hydration state of the user.

A second experiment manipulating hydration status was conducted to simulate isotonic dehydration as well as hyper-hydration. Changes in circulating volume were induced with lower body negative pressure or lower body positive pressure. Lower body pressure was varied from −30 mmHg to +40 mmHg in discrete stages. Thirteen healthy male subjects, ranging in age from 19 to 39 years, were recruited to take part in the study.

Average heart rate (HR), mean arterial pressure (MAP), and ejection time as a function of lower body pressure (LBP) are shown FIG. 37. Lighter lines represent each individual subject while darker lines with cross denotes the group mean. Effects of lower LBP on HR (FIG. 37A) and MAP (FIG. 37B) were relatively subtle and heterogeneous across the sample. Using a repeated measures analysis of variance (ANOVA) test to assess differences across conditions, no significant changes in HR were found at the group level (F_{4,12}=0.7, p=0.48), while MAP showed a trend toward increasing with LBP (F_{4,12}=2.3, p=0.069). In contrast, negative LBP decreased ejection time in a graded fashion and positive LBP slightly increased (F_{4,12}=55.0, p<10⁻¹⁶). Within-subject one-way ANOVAs confirmed a strong effect in all subjects (p<0.005, corrected for multiple comparisons). These results stress the unique value of ejection time to detect changes in hydration status and by comparison, the relative insensitivity of HR and MAP to mild changes in circulating volume.

### Hydration Assessment While Exercising.

An exercise study without fluid replacement was conducted. At the beginning, middle and end of the exercise period the subject was asked to explicitly vary their heart rate over a pre-defined range by changing power output. FIG. 37 is a plot of the results obtained. The aortic valve time sequence information was recorded using a near-infrared PPG sensor on the tip of the finger. Examination of the plots shows the very distinct grouping of points as the subject became increasingly dehydrated. Moreover, the data demonstrates the ability to determine hydration status while subjects experience heart rate changes during a significant exertion.

Hydration Assessment with Positional Change.

FIG. 39 demonstrates the value of using body position changes to enhance or augment the hydration assessment. The exercise-induced dehydration study protocol was executed as before, but the subject moved through supine, sitting and standing body positions during each measurement period. The sequence of aortic valve opening and closing was obtained using a near-infrared PPG sensor placed at the base of the finger. The heart rate and ejection time measurements during each period and body position are shown in FIG. 39A. As seen in the figure, the initial baseline measurements show minimal change in heart rate and an ejection time change of 60 ms. However, with change in hydration of 2.5%, the degree of change due to position is significantly larger with a heart rate change ˜16 beats per minute and a change in ejection time of 115 ms. As the subject recovers, the degree of posture-induced changes decreases until near-baseline changes are observed.

The addition of positional change information adds additional information on hydration status that can be effectively incorporated as illustrated in FIG. 18. FIG. 39B shows the output of a hydration determination model that linearly combines changes in IBI and ET from supine to standing positions to provide a hydration assessment. The ability to obtain positional change information can occur passively as a user exits from a bed in the morning or moves from a desk to a standing position when at work.

### Scenarios for System Use

The following use cases are provided to help illustrate the value and inventive nature of the system. The presented use cases comprise limited examples and one of skill in the art will recognize additional scenarios where the invention is of use.

Military Operations. An embodiment of the current invention can be used for military personnel who are at risk for dehydration due to body armor requirements and overall physiological stress due to military operations. Military personnel don significant protective gear in extreme environmental conditions that can include the risk of combat. Collectively, these conditions can place enormous physiologic stress on the body with physical and cognitive consequences. One can appreciate the problem by considering military units operating in the Middle East. Despite a focus on water consumption to keep soldiers in good health, combat conditions can create significant distractions that when coupled with 110° F. temperatures create an ideal environment for decreased physiological performance. Dehydration also puts soldiers at greater risk for loss of life should they become injured in combat; in the event of hemorrhage (isotonic dehydration), the body's ability to maintain sufficient perfusion to vital organs is severely compromised when baseline vascular volume is already reduced. The described hydration assessment system can provide oversight of vascular volume with no additional burdens in soldiers' time, behavior, or gear. Thus, the invention has significant value to military personnel.

Elderly Patient Assessment. An embodiment of the current invention has applicability in monitoring the hydration status in the elderly due to limited reserves and the consequences of a fall or loss of cognitive function. With increasing age, body water content decreases, the risk for dehydration increases, and the consequences become more serious. Additionally, the “drink to thirst” mechanism loses effectiveness. Dehydration has been associated with increased mortality rates among hospitalized older adults and can precipitate emergency hospitalization and increases the risk of repeated stays in hospital. Dehydration is a frequent cause of hospitalization of older adults and one of the ten most frequent diagnoses responsible for hospitalization in the United-States. Evidence suggests high dehydration rates of elderly patients within hospitals and other health care institutions and is considered a form or abuse. The impact of dehydration is associated with various morbidities, such as impaired cognition or acute confusion, falling or constipation. Dehydration has been linked to increased risk of stroke and myocardial infarction. The expenditures linked to dehydration are significant as evidenced in a 1999 study that estimated the avoidable costs of hospitalizations due to dehydration at $1.14 billion. Insufficient fluid intakes result from limitations such as reduced swallowing capacity, decreased mobility, or comprehension and communication disorders.

Assessment of hydration in the elderly demonstrates the value of the invention for the avoidance of falls, cognitive lapses, increased risk of cardiovascular events, and kidney stone developments. In this scenario the feedback system can be configured to report status information to a family member, a remote monitoring service, or to a nursing station in an assisted living setting. The system can use the postural transitions from sleeping to sitting to standing as a method for accessing aortic valve timing under three different venous return conditions. The ability to compare day-to-day trends for a single individual enables the detection of small perturbations that can be important in the physiologically fragile individual.

Daily Living. An embodiment of the current invention has general applicability to the general population. For the purpose of illustration consider a business executive on international travel. The dry air used to pressurize jet airplane cabins coupled with limited beverage service leads to volume depletion. The executive can use a hydration assessment system to effectively ensure that fluid intake is appropriate. The burden on the user is minimal and only requires the executive to don a ring or other wearable device such that aortic valve opening and closing information is obtained.

Post-Exercise Assessment. An embodiment of the current invention also has applicability for any athlete looking to recover for exercise. An example scenario can involve a vigorous skiing day with friends. The ability of an individual to self-assess their hydration status can be impeded due to several factors, e.g., the dryness of high mountain air, increased respiratory rate due to decreased oxygen concentration resulting in increased respiratory fluid loss, perspiration on very challenging (“black diamond”) runs, and after-ski consumption of alcohol, a known diuretic. The hydration assessment system can provide information for optimal fluid intake and recovery so that the second day of the ski trip is as enjoyable as the first. Other use case scenarios include back-to-back soccer games, tennis tournaments, multi-day sailing tournaments, 18 holes in the holes of golf in the Arizona sun and training for a marathon.

Within-Exercise Assessment. An embodiment of the current invention can be used by athletes for hydration maintenance during exercise. Use scenarios include any endurance events where the “drink-to-thirst” approach has been shown to ineffective. The Hawaii Ironman is an event known for epic collapses due to hydration mismanagement. A similar event known for hydration complexities is the La Ruta mountain bike race across Costa Rica. Many North America athletes travel to Costa Rica to participate in the event but have little experience with the tropical humid environment and are also concerned with drinking untreated water. The event is a significant endurance event with the cycling time often exceeding 4 hours. The ability to use the physiological assessment system to determine circulating volume during the event can have profound value, allowing athletes to maintain hydration at baseline levels throughout the event. The system can provide real-time assessments of hydration status, displayed on a standard cyclometer device, as well as alerts if circulation volume was changing rapidly or progressing to dangerously low levels.

Multiple Rings for Convenience. In ring-type embodiments, the small device size significantly limits battery size and power capacity. As discussed above, processes can be implemented for battery conservation. Alternately or in addition, a set of two or more devices may be provided to the user, such that one device can be charging while another device is worn. FIG. 40A shows an example of a pair of rings. One ring may be worn during the day, while the other is worn at night. Alternatively, one ring may be worn until a notification of low battery is provided, encouraging the user to “swap” the ring for the second ring. As shown in FIG. 40B, the pair of rings may have appearances distinct from each other, which facilitate ring swapping. Such embodiments provide a convenient solution for users to continuously wear a hydration determination device.

### Operational Embodiment

### General Architecture.

FIG. 41 depicts an exemplary control architecture for a situationally-aware hydration-monitoring device configured to (i) predict the probability and rate of imminent body-water change, (ii) invoke burst sampling when appropriate, and (iii) confirm that acquired data are of sufficient fidelity to quantify hydration status. Unless otherwise stated, the arrows in the figure represent digital-message flow between firmware modules executed on a common micro-controller unit (MCU); however, individual blocks may alternatively reside on a companion application processor, mobile device or in cloud infrastructure without departing from the inventive concept.

Measurement Sub-system (4101) includes a multi-modal sensor suite that delivers parameters that are empirically or mechanistically linked to total-body-water dynamics, including heart-rate (HR), skin or external temperature, respiratory rate, inertial-derived activity level or movement class, geolocation/time metadata, and heart-rate variability (HRV). Each metric is time-stamped and queued for probabilistic assessment.

System to Determine Probability and Rate of Hydration Change (4102) is composed of probability-engine module that uses a Bayesian or machine-learned estimator to ingest the aforementioned metrics and outputs two scalar values: (i) Hydration-Change Probability (HCP). The estimator implicitly weights variables reflecting internal heat production (elevated HR, rising skin temperature) and external heat load (ambient temperature, radiant index, altitude) to ensure that both metabolic and environmental dehydration risks are captured.

Probability-Above-Threshold Trigger: If HCP exceeds a programmable threshold or the rate of estimated hydration change is high, the burst-sampling trigger system is notified. If the probability is low, the measurement subsystem, 4101, continues monitoring and a baseline measurement cadence is continued.

Trigger for Burst Sampling Controller receives three inputs and processes these inputs to determine if a burst sampling should be initiated. The Probability-Above-Threshold Trigger provides an input based on the probability of hydration change.

The Adequacy of Hydration Assessment Trigger system uses direct hydration measurements to determine if the user is experiencing hydration changes. This is in contrast to the System to Determine the Probability of Hydration Change uses a probability estimate. The Adequacy of Hydration Assessment Trigger system is especially valuable during rehydration as many of the internal and external heat load metrics may have returned to normal, but the user still has a altered body water state. The Measurement Cadence Trigger ensures that there is a baseline measurement cadence. For example, the system may male hydration measurements evert hour during sleeping and every 30 minutes when awake. This time-based trigger guarantees longitudinal trend data.

The Trigger for Burst Sampling Controller integrates these various inputs to determine is a burst sampling should be initiated. The process accounts for the timing of the last measurement, the actual rate of hydration change and the probability of a hydration change to ensure that the hydration status of the user is adequately measured. If a hydration measurement is needed the burst controller initiates the process and selects one or more of the following operational adaptations to facilitate adequate data collection.

Adjust sampling frequency is the process of changing the sampling frequency from a baseline to a level that enables adequate calculation of left ventricular ejection time. A sampling frequency of greater than 100 Hz is desired. An increase in sampling frequency is typically the most desired change versus other operating parameter. Because each light pulse remains brief, raising the sampling frequency adds only a small increment to average power and lets the electronics quickly return to a low-power state. By contrast, boosting LED drive current or activating extra emitters scales the energy of every pulse, creating a nearly proportional increase in total battery draw.

Adjust System Operational Parameters is a modality that can be initiated by the burst sampling controller. Employing additional emitters or detectors, or driving existing LEDs at higher current, increases photon flux and collection area, thereby boosting signal-to-noise ratio and expanding the dynamic range available to the ADC. However, each extra emitter introduces an additive current pulse, and every additional detector channel keeps the analog front-end and converter active for longer intervals, so the average power draw scales almost linearly with the number of devices and the drive amplitude. Accordingly, while these measures materially improve measurement quality, they do so at the predictable cost of reduced battery longevity.

Recommend Transmural-Pressure Changes. The burst-sampling controller promotes a temporary reduction in transmural pressure by reducing the internal circumference of the finger-worn device. As illustrated in FIGS. 22-30, this dimensional change may be affected through user-initiated deformation mechanisms such as a ratcheting click band, a cam-driven rotary collar, a screw-thread constrictor, or interlocking shell segments that snap to a smaller diameter. Lowering transmural pressure delivers two synergistic benefits: (i) superficial veins are partially collapsed, attenuating motion-related venous-volume artefacts, and (ii) arterial pulse-wave amplitude is magnified, sharpening systolic upstrokes and valve landmarks for higher-fidelity optical sensing. Because these mechanisms rely on passive mechanical reconfiguration—rather than sustained motor actuation or increased LED drive—they impose essentially no incremental battery load. The user may elect to maintain the ring in its compressed configuration throughout an activity (e.g., extended dehydration monitoring) or engage it only during a brief burst-sampling window, after which the device can be returned to its relaxed, everyday-comfort diameter. Adequacy of Measured Data is a computational and quality control system that evaluates the

signal-to-noise ratio, motion artefact index, and feature-extraction confidence for each burst. If the data fail to meet predefined acceptance criteria, the controller issues a “Changes to Burst Sampling” command, requesting that busrt sampling controller make additional modifications that may include dynamically modifying operational parameters (e.g., extend burst duration, further tighten the ring, or postpone sampling to the next motion-free interval).

In certain embodiments, thermoregulatory signals and contextual data collected by the wearable device are transmitted via Bluetooth®, Wi-Fi, cellular, or other wireless protocols to an external processing device, such as a smartphone, tablet, or cloud-based server. The external device performs intensive computational tasks, including probability-score estimation, hydration modeling, and predictive analytics, and subsequently relays hydration-status information or actionable recommendations back to the wearable or a user-facing interface.

In these off-device embodiments, the external processing device also computes a hydration-change probability score from the transmitted contextual-sensor data and, when that score exceeds a configurable threshold, returns adaptive-control instructions—such as commands to raise optical sampling frequency to at least 100 Hz or to shorten measurement cadence—to the wearable device.

The processing of the plethysmographic data and the hydration determination system can also be done on external processing devices. Specifically, if a ring system is the mechanism for acquisition of plethysmographic data, then the majority of energy consuming processing will be done outside the wearable hydration-sensor housing in an effort to extend battery life.

### Example Scenario (Finger Sensor for Mountain-Bike Session)

The following day-in-the-life example is offered solely to illustrate how the situationally-aware architecture of FIG. 41 delivers an adaptive hydration-monitoring experience whose data fidelity scales with the moment-to-moment probability of a physiologically meaningful change in body water. Every numerical value—sampling rate, optical wavelength, probability threshold, and timing interval—is provided for explanatory clarity and should not be construed as limiting.

At 07:00, the ring-based device begins the day in its *standard-fidelity* state. A Baseline Measurement Trigger obliges the system to acquire one hydration measurement every thirty minutes while the user is awake. Each baseline hydration measurement is executed as a short burst: a thirty-second photoplethysmogram collected at 200 hertz is a twenty-five hertz with a single 940 nm emitter and one detector. Immediately after acquisition, the signal is converted into an estimated hydration status and forwarded-together with contextual variables such as heart rate, heart-rate variability, distal skin temperature, respiratory rate, on-body activity level, ambient temperature provided by the paired smartphone, and geolocation-to the System to Determine Probability of Hydration Change. Because the morning values suggest thermal neutrality and metabolic rest, the algorithm computes a hydration-change probability of 0.05, well below the decision threshold and control returns to the Continue Monitoring branch of FIG. 41 without changing the device's behavior.

Late in the afternoon, at 16:30, two new pieces of contextual information arrive: the smartphone detects arrival within a trail-head geofence, and a calendar event labelled “Evening mountain-bike ride” is scheduled to begin in thirty minutes. Incorporating these cues, the probability engine raises its estimate to 0.18, surpassing the threshold of 1.0. That single change is enough to activate the Probability-Above-Threshold Trigger, which transmits an affirmative signal to the Burst-Sampling Controller. The controller responds by tightening the measurement cadence to every ten minutes, enabling an auxiliary 940 nm emitter for greater intensity and tissue illumination.

When the ride starts at 17:05 the inertial sensor embedded in the ring records rhythmic accelerations characteristic of pedaling. Heart rate climbs forty per cent above the morning baseline, and skin temperature rises by 1.2 degrees Celsius. The probability engine now predicts an imminent hydration loss. The Adequacy of Hydration Assessment Trigger, which bases its judgement on measured hydration values and their rate of change rather than on indirect predictors, therefore returns a negative decision, as hydration has not changed at the start of the ride. However, later in the ride, dehydration begins and the Adequacy of Hydration Assessment Trigger becomes positive. Because both the probability trigger and the adequacy trigger are now active, the Burst-Sampling Controller increases the measurement frequency to every 5 minutes and activates the use of green LEDs at (550 nm).

To maximize signal quality during motion, the controller issues a recommendation for a reduce transmural pressure. A brief haptic pulse instructs the user to actuate the ring's mechanical constrictor by altering the ring configuration as shown in FIG. 28, resulting in a decrease of the ring's internal circumference. The resulting compression and partial collapse of superficial veins diminishes motion-induced venous artefact and simultaneously boosts arterial pulse-wave amplitude, improving the optical resolution of aortic-valve opening and closing without any additional battery drain.

The mountain-bike session ends at 19:10. Although the activity sensor now reports only low-level motion and heart rate is trending toward baseline, the most recent hydration measurements still indicate a deficit exceeding one per cent of total body water. For that reason, the adequacy trigger remains affirmative even though the predictive probability has already fallen below threshold. The system therefore maintains burst operation but relaxes its parameters.

By 21:00 sequential measurements confirm that total body water has returned to near pre-exercise values via fluid intake and that the rate of change has stabilized. Both triggers now evaluate to negative, enabling the Burst-Sampling Controller to revert the device to standard-fidelity operation under exclusive control of the Baseline Measurement Trigger.

The mountain biking example was selected at it demonstrates the value of situational awareness. The system proactive sense that an exercise was likely bu geolocation and calendar, and then used the initial changes in physiology to proactively initiate hydration measurements before significant hydration changes had occurred. The result is a system that continuously modulates measurement cadence, optical-sensor configuration, and mechanical transmural-pressure settings—always in proportion to the inferred or observed risk of hydration change—so that the user receives data whose fidelity is consistent with the physiological situation.

### Example Scenario (Wristwatch Sensor for Medication Change and Nausea)

The following multi-day scenario, which draws extensively on the user's paired smartphone for contextual data and safety messaging, illustrates how the adaptive architecture of FIG. 41 safeguards an older adult whose thirst perception has been blunted by a recent antihypertensive adjustment. All numerical values and thresholds are illustrative only.

Three days earlier a seventy-two-year-old user replaced a long-standing angiotensin-receptor blocker with a low-dose thiazide-like diuretic. By blocking sodium-chloride re-uptake in the distal convoluted tubule, the drug promotes natriuresis, lowers plasma osmolality, and dampens the hypothalamic thirst drive. Mild nausea compounds the tendency to drink less. Feeling unwell, the user forgets to activate the apartment's air-conditioning on an unseasonably warm spring day, so indoor temperature rises steadily through the afternoon.

At 06:30 the Baseline Measurement Trigger in the ring-based hydration monitor initiates a one-minute burst at 150 hertz with a single 530-nanometre emitter and one photodetector. Hydration status—expressed as percentage deviation from the user's personal euvolaemic reference—is −0.3% TBW, well inside normal variability. Heart rate, heart-rate variability, distal skin temperature, respiratory rate, activity level, and smartphone-derived ambient temperature feed the System to Determine Probability of Hydration Change, which returns a probability of 0.05, far below the decision threshold of 0.40. The device therefore remains in standard-fidelity sampling, scheduling bursts every thirty minutes while the user is awake and every ninety minutes during sleep or customary afternoon rest.

Morning nausea suppresses both appetite and fluid intake. By 13:00 three additional bursts have been recorded. The most recent measurement shows a −2.0% TBW deviation—roughly −1% of body weight—and the 90-minute slope is −1.3% TBW, equivalent to −0.014% per minute. Although the predicted probability is still below threshold, the Adequacy of Hydration Assessment Trigger, which relies on direct measurements and their first derivative, classifies the trend as clinically relevant and instructs the Burst-Sampling Controller to tighten measurement cadence to every fifteen minutes.

After a light lunch the user lies down. Low blood volume and the vasodilatory effect of the diuretic produce a small arterial pulse at the measurement site, degrading signal-to-noise. The Adequacy of Measured Data subsystem therefore requests an optical upgrade: the Burst-Sampling Controller activates both green (530 nm) and infrared (850 nm) emitters and brings online two additional photodetectors spaced evenly along the ring's inner circumference. These changes restore plethysmographic quality sufficient for reliable hydration determinations.

When the user wakes at 16:45—lethargic but not overtly dizzy—three hours of high-resolution monitoring document a progressive deficit now at −4% TBW. Because total body water in men over seventy averages about fifty per cent of body weight, the present deficit corresponds to a −2% weight loss, a threshold widely accepted as clinically significant dehydration and a precursor to orthostatic hypotension and falls.

The smartphone application elevates its alert level, recommending 200 ml of electrolyte solution every fifteen minutes until further notice. For a seventy-kilogram individual, the −2% body-weight deficit represents a 1.4-litre fluid shortfall, implying a rehydration interval of roughly 100 minutes. The user's nausea leads to non-compliance, and the next burst confirms passage beyond the −4% TBW mark.

Crossing that threshold triggers the system's high-alert protocol: the smartphone sounds a persistent alarm, pushes notifications to the designated care team, and advises the user to remain seated or in bed to mitigate fall risk.

At 17:20 medical assistance arrives and administers one liter of intravenous fluid, reducing the deficit to approximately −2% TBW (about −1% body weight). Physicians discontinue the new diuretic and instruct the user to continue oral fluids until euvolemia is restored.

The hydration monitor remains in its heightened state, maintaining 15-minute bursts while confirming the upward trajectory toward baseline. Once two consecutive measurements fall within −1% TBW and the slope flattens, control logic will automatically step down first to the intermediate cadence and eventually back to the baseline schedule.

In this scenario the user's suppressed thirst and mild malaise provided no intuitive warning, yet the wearable and its companion phone detected the emerging dehydration, escalated both measurement rate and optical operating parameters to compensate for a shrinking pulse amplitude, and delivered increasingly urgent prompts until corrective action occurred. By preventing progression to severe hypotension, the system reduced the likelihood of a dehydration-related fall—a particularly serious hazard in the elderly due to the potential of head trauma and hip fractures.

### Example Scenario (Temple-Based Sensors for Soccer)

An athletic embodiment of the temple-based hydration-measurement system is realized in a pair of performance sunglasses designed for recreational and competitive soccer. The right-hand temple contains an optoelectronic module that mirrors the architecture disclosed in FIG. 42 of the specification. A near-infrared emitter (940 nm), 4203, flanks a detector. 4201, and a second emitter (530 nm) is more proximal to the detector (4202) to provide a shallow sampling path. A molded, low-durometer gasket shields the detector from ambient light and distributes contact pressure evenly across the post-auricular skin. A miniature speaker, 4205, embedded in the lower temple delivers spoken or tone prompts without occluding the ear. Data is transferred via Bluetooth from the eyewear to a sideline smartphone or tablet for off-device processing. Opportunistic burst transmission during proximity leverages moments when the player passes within 10-30 meters of a sideline receiver to rapidly offload buffered data. This approach preserves battery life and ensures high-fidelity data transfer without requiring continuous connection during active play.

When the glasses are donned pre-match, the Measurement Subsystem samples heart rate, three-axis acceleration, skin and ambient temperature, and device geolocation at a low-energy baseline. These data are processed by the Probability of Hydration Change System that weights internal heat load (heart-rate elevation, skin-temperature rise) and external heat load (field temperature, radiant index) to predict the probability of increase fluid loss. The totality of factors present during warm-up results in a high probability of fluid loss, and the Cadence Controls system is notified. The Cadence-Control System increases the measurement cadence to a desired measurement goal of every 10 minutes. The user now has per-game measurement values, and the monitoring system is fully active for the start of the game.

Soccer is defined by sprints followed by periods of lower activity, especially when the ball is on the opposite field. Thus, motion may decrease almost instantly, but heart rate may remain elevated. The resting heart rate may not return to baseline until halftime or after the game. Thus, the Operational-Control System will automatically raise the plethysmographic sampling rate to 200 Hz to preserve the temporal resolution of left-ventricular ejection time under tachycardia. The Operational-Control System invokes Motion-Scenario-Opportunity (MSO) Sampling so that optical sampling bursts coincide with natural lulls—e.g., throw-ins, goal-kicks, or a free-kick setup—when head movement subsides. The burst continues until excessive motion is detected or until adequate plethysmographic data is obtained. The Operational-Control System, Plethysmographic Data Acquisition, and Measurement Suitability System work together to acquire the plethysmographic data. The process could result in 5 data acquisition periods totaling 30 seconds to create a measurement sample for hydration determination.

Hydration status is the percentage deviation from a personal baseline of 57% total body water (TBW), a representative value for well-trained adult males. A cumulative loss that equals 1% of body mass corresponds to ≈1.75% TBW for this user. When the integrated deficit reaches 1.5% TBW, the open-ear speakers provide the user with a hydration status and recommend fluid consumption. If the deficit creeps to 3% TBW, a second alert pings both the player and the paired device, enabling a coach or parent to intervene before performance or cognition deteriorates.

The system remains active as the user rehydrates because tournament schedules often comprise two or more games into a summer afternoon.

Throughout the game, the Operational-Control System may recommend the user reduce the transmural pressure to increase the arterial pulse amplitude. A discrete voice cue, “Press right temple for 10 seconds”, asks the user press the frame gently against the temple during the next dead-ball interval, momentarily compressing superficial veins and improving signal-to-noise.

Following the match, the cadence control system relaxes the measurement cadence in proportion to the descending probability score.

This narrative preserves every functional element earlier disclosed for the temple-based hydration monitor—situational awareness, predictive assessment, MSO sampling, adaptive optical parameters, transmural-pressure modulation, and multi-modal feedback—while casting them into a cohesive example suitable for direct insertion into a patent specification.

The described embodiment demonstrates one practical implementation of the general ear-temple sampling concept. It illustrates how the anatomical advantages of the site can be combined with low-power optoelectronics, miniature power sources, and wireless communication to produce a continuous, motion-robust hydration monitor that integrates seamlessly into conventional eyewear.

Each of the publications referred to herein are incorporated herein by reference.

Those skilled in the art will recognize that the present invention can be manifested in a variety of forms other than the specific embodiments described and contemplated herein. Accordingly, departures in form and detail can be made without departing from the scope and spirit of the present invention as described in the appended claims.

While the concepts of the present disclosure are susceptible to various modifications and alternative forms, specific embodiments thereof have been shown by way of example in the drawings and described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described can include a particular feature, structure, or characteristic, but not every embodiment must necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments can be implemented, in some cases, in hardware, firmware, software, or any combination thereof. The disclosed embodiments can also be implemented as instructions carried by or stored on a transitory or non-transitory machine-readable (e.g., computer-readable) storage medium, which can be read and executed by one or more processors. A machine-readable storage medium can be embodied as any storage device, mechanism, or other physical structure for storing or transmitting information in a form readable by a machine (e.g., a volatile or non-volatile memory, a media disc, or other media device).

In the drawings, some structural or method features are shown in specific arrangements and/or orderings. However, it should be appreciated that such specific arrangements and/or orderings might not be required. Rather, in some embodiments, such features can be arranged in a different manner and/or order than shown in the illustrative figures. Additionally, the inclusion of a structural or method feature in a particular figure is not meant to imply that such feature is required in all embodiments and, in some embodiments, might not be included or might be combined with other features.

What is claimed is:

1. An apparatus for determining the hydration status of a user, comprising:
(a) a sensor housing, configured to be worn by the user without interfering in the activities of daily living;
(b) an optical sensor system comprising (i) one or more optical emitters mounted with the sensor housing such that light emitted by the one or more emitters is directed toward the skin of the user and (ii) one or more detectors mounted with the sensor housing such that the one or more detectors produce a detector signal representative of light reaching the detectors from one or more emitters after the light has interacted with tissue of the user, configured to detect physiological signals indicative of opening and closing of the user's aortic valve;
(c) a cadence control system, comprising a situational awareness system or a predictive assessment system or both, configured to indicate a measurement cadence;
(d) an optical sampling control system responsive to the cadence control system configured to operate the one or more emitters and the one or more detectors at a cadence determined by the cadence control system;
(e) a plethysmographic analysis system responsive to the detector signal and configured to determine an interbeat time interval between successive openings of the user's aortic valve, and an ejection time interval between opening and closing of the user's aortic valve;
(f) a hydration determination system configured to determine the hydration status of the user from the interbeat time interval and the ejection time interval;
(g) a feedback system configured to provide feedback.

2. The apparatus of claim 1, wherein the cadence control system comprises a situational awareness system.

3. The apparatus of claim 1, wherein the cadence control system comprises a predictive assessment system.

4. The apparatus of claim 2, wherein the situational awareness system is configured to indicate an increased cadence when the situational awareness system determines, from evaluation of thermoregulatory signals and environmental factors, that the user's hydration is likely to change more than a predetermined threshold before the next hydration measurement at the current cadence.

5. The apparatus of claim 3, wherein the predictive assessment system is configured to indicate an increased cadence when the predictive assessment system determines, from evaluation of the rate of change of the user's historical hydration, that the user's hydration is likely to change more than a predetermined threshold before the next hydration measurement at the current cadence.

6. The apparatus of claim 1, wherein the sensor housing comprises a ring.

7. The apparatus of claim 1, wherein the sensor housing comprises eyeglasses, and wherein the optical sensor system is mounted with one or both earpieces of the eyeglasses.

8. The apparatus of claim 1, further comprising a measurement suitability system, wherein optical sampling control system is responsive to the measurement suitability system, and wherein the measurement suitability system is configured to do one or more of: determine the sampling frequency; determine the optical acquisition profile, suggest a transmural pressure for the next hydration measurement; determine the use of motion-scenario-opportunity sampling.

9. A method of adaptive hydration monitoring, comprising:
(a) measuring, with a wearable device, at least one contextual parameter comprising
(i) one or more thermoregulatory signals selected from the group consisting of heart-rate, skin temperature, and activity level, or
(ii) one or more environmental factors selected from the group consisting of ambient temperature and ambient humidity;
(b) computing, from at least one contextual parameter, a probability of hydration change for a user;
(c) comparing the probability of hydration change to a predetermined threshold indicative of increased dehydration risk;
(d) acquiring plethysmographic data, when the probability exceeds the predetermined threshold, at a sampling frequency of at least 100 Hz;
(e) extracting, from the plethysmographic data,
(i) an inter-beat time interval representing successive aortic-valve openings, and
(ii) an ejection time interval representing an opening-to-closing event of the aortic valve;
(f) determining a hydration status by processing the inter-beat time interval and the ejection time interval with a hydration determination model; and
(g) providing feedback indicative of the hydration status to the user or to an external computing device.

10. The method of claim 9, further comprising;
(a) estimating the user's total-body-water level from contextual parameters, and
(b) increasing a measurement cadence of the wearable device so that a successive hydration determination will be made before the user's total-body-water level is estimated to change by more than 0.5 percent.

11. The method of claim 9, further comprising:
(a) predicting, from a rate of hydration change a time period at which the probability of hydration change is expected to exceed a predetermined hydration-change threshold; and
(b) initiating a hydration determination at a scheduled time determined from the time period.

12. The method of claim 9, further comprising;
(a) estimating the rate of hydration change from at least two hydration measurements; and
(b) determining a time period at which the estimated hydration will exceed a predetermined hydration-change threshold; and
(c) initiating a hydration determination before the determined time period.

13. A situationally-aware, adaptive hydration-monitoring apparatus, comprising:
(a) a wearable housing;
(b) an optical sensor system within the housing, comprising at least one light emitter and at least one photodetector, the optical sensor system being configured to generate plethysmographic signals;
(c) a contextual-sensor suite including at least one thermoregulatory parameter selected from heart-rate, respiratory-rate, activity level or skin-temperature sensors;
(d) a probability-engine module, implemented by one or more processors, configured to fuse signals from the contextual-sensor suite and output a hydration-change probability score;
(e) a measurement cadence controller configured to compare the probability score with a programmable threshold and, when the probability score exceeds the threshold, activate the operational control system to acquire plethysmographic data by using the optical sensor system at an increased cadence;
(f) a plethysmographic analysis system configured to extract an ejection time interval and an interbeat interval from the plethysmographic signals; and
(g) a hydration determination system configured to determine the hydration status of the user from the interbeat time interval and the ejection time interval;
(h) a feedback system configured to provide feedback.

14. The apparatus of claim 13, wherein the wearable housing comprises an eyeglass frame having a temple arm terminating in a temple tip region designed to contact post-auricular skin; having an optical sensor system embedded in the temple tip, comprising at least one light emitter and at least one photodetector arranged for reflectance plethysmography.
